(19) United States
(12) Patent Application Publication
(10) Pub. No.: US 2024/0166623 A1
(43) Pub. Date: May 23, 2024

(54) NOVEL PROCESS

(71) Applicant: ARTIOS PHARMA LIMITED, Cambridge (GB)

(72) Inventors: Martin Lee STOCKLEY, Cambridge (GB); Robert HEALD, Cambridge (GB); David LATHBURY, Cambridge (GB); Pingao YANG, Cambridge (GB); Qinjin WANG, Cambridge (GB); Yanjie FU, Cambridge (GB); Mark Theodoor VERHAAR, Cambridge (GB); Fuxu LIAO, Cambridge (GB); John LIDDON, Cambridge (GB); Matthew LLOYD, Cambridge (GB); TANG Xiaoping, Cambridge (GB); Zarrin ANSARI, Cambridge (GB); Stephen KEEN, Cambridge (GB)

(73) Assignee: ARTIOS PHARMA LIMITED, Cambridge, EN (GB)

(21) Appl. No.: 18/264,079

(22) PCT Filed: Feb. 7, 2022
(86) PCT No.: PCT/GB2022/050310

§ 371 Date: Aug. 3, 2023

(30) Foreign Application Priority Data
Feb. 7, 2021 (CN) 202110166985.5
Feb. 8, 2021 (GB) 2101715.7

(51) Int. Cl. C07D 401/04; C07C 211/48; C07D 207/12

(52) U.S. Cl. CPC C07D 401/04; C07C 211/48; C07D 207/12; C07B 2200/05

(57) ABSTRACT

The invention relates to a process for preparing heterocyclic amide derivatives, to novel polymorphic forms obtained from said process and the use of said polymorphic forms for use in the treatment and prophylaxis of cancer.

### FIELD OF THE INVENTION

The invention relates to a process for preparing heterocyclic amide derivatives, to novel polymorphic forms obtained from said process and the use of said polymorphic forms for use in the treatment and prophylaxis of cancer.

### BACKGROUND OF THE INVENTION

Robust repair of DNA double-strand breaks (DSBs) is essential for the maintenance of genome stability and cell viability. DSBs can be repaired by one of three main pathways:

homologous recombination (HR), non-homologous end-joining (NHEJ) and alternative NHEJ (alt-NHEJ). Microhomology-mediated end-joining (MMEJ) is the most well characterised alt-NHEJ mechanism. HR-mediated repair is a high-fidelity mechanism essential for accurate error-free repair, preventing cancer-predisposing genomic stability. Conversely, NHEJ and MMEJ are error-prone pathways that can leave mutational scars at the site of repair. MMEJ can function parallel to both HR and NHEJ pathways (Truong et al. PNAS 2013, 110 (19), 7720-7725).

The survival of cancer cells, unlike normal cells, is often dependent on the mis-regulation of DNA damage response (DDR) pathways. For example, an increased dependency on one pathway (often mutagenic) to cope with either the inactivation of another one, or the enhanced replication stress resulting from increased proliferation. An aberrant DDR can also sensitise cancer cells to specific types of DNA damage, thus, defective DDR can be exploited to develop targeted cancer therapies. Crucially, cancer cells with impairment or inactivation of HR and NHEJ become hyper-dependent on MMEJ-mediated DNA repair. Genetic, cell biological and biochemical data have identified Pole (UniProtKB-O75417 (DPOLQ_HUMAN) as the key protein in MMEJ (Kent et al. Nature Structural & Molecular Biology (2015), 22(3), 230-237, Mateos-Gomez et al. Nature (2015), 518(7538), 254-257). Polθ is multifunctional enzyme, which comprises an N-terminal helicase domain (SF2 HEL308-type) and a C-terminal low-fidelity DNA polymerase domain (A-type) (Wood & Doublié DNA Repair (2016), 44, 22-32). Both domains have been shown to have concerted mechanistic functions in MMEJ. The helicase domain mediates the removal of RPA protein from ssDNA ends and stimulates annealing. The polymerase domain extends the ssDNA ends and fills the remaining gaps.

Therapeutic inactivation of Polθ would thus disable the ability of cells to perform MMEJ and provide a novel targeted strategy in an array of defined tumour contexts. Firstly, Polθ has been shown to be essential for the survival of HR-defective (HRD) cells (e.g. synthetic lethal with FA/BRCA-deficiency) and is up-regulated in HRD tumour cell lines (Ceccaldi et al. Nature (2015), 518(7538), 258-262). In vivo studies also show that Polθ is significantly over-expressed in subsets of HRD ovarian, uterine and breast cancers with associated poor prognosis (Higgins et al. Oncotarget (2010), 1, 175-184, Lemée et al. PNAS (2010), 107(30), 13390-13395, Ceccaldi et al. (2015), supra). Importantly, Polθ is largely repressed in normal tissues but has been shown to be upregulated in matched cancer samples thus correlating elevated expression with disease (Kawamura et al. International Journal of Cancer (2004), 109(1), 9-16). Secondly, its suppression or inhibition confers radio-sensitivity in tumour cells. Finally, Polθ inhibition could conceivably prevent the MMEJ-dependent functional reversion of BRCA2 mutations that underlies the emergence of cisplatin and PARPi resistance in tumours.

There is therefore a need to provide effective Polθ inhibitors for the treatment of cancer.

### SUMMARY OF THE INVENTION

According to a first aspect of the invention, there is provided a process for preparing a compound of formula (I):

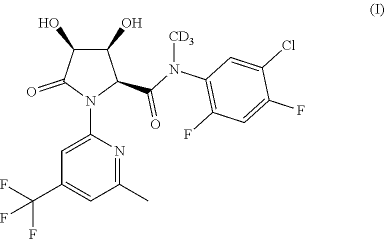 [SMILES: [2H]C([2H])([2H])N(C(=O)[C@@H]1[C@H](O)[C@H](O)C(=O)N1c1cc(C(F)(F)F)cc(C)n1)c1cc(Cl)c(F)cc1F]

which comprises treating a compound of formula (XX):

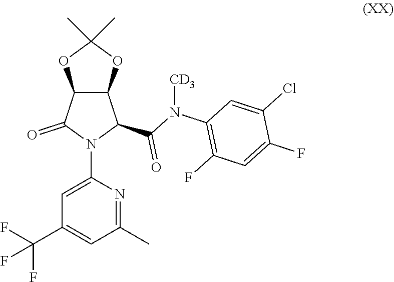 [SMILES: [2H]C([2H])([2H])N(C(=O)[C@@H]1[C@@H]2OC(C)(C)O[C@@H]2C(=O)N1c1cc(C(F)(F)F)cc(C)n1)c1cc(Cl)c(F)cc1F]

with a Lewis acid in the presence of a scavenger agent.

According to a further aspect of the invention, there is provided a compound of formula (I) obtainable from the process as defined herein.

According to a further aspect of the invention, there is provided a pharmaceutical composition comprising a compound of formula (I) as defined herein, in combination with one or more therapeutic agents.

According to a further aspect of the invention, there is provided a compound of formula (I) as defined herein for use in therapy.

According to a further aspect of the invention, there is provided a compound of formula (I) as defined herein for use in the prophylaxis or treatment of cancer.

### BRIEF DESCRIPTION OF THE FIGURES

FIG. 1: X-Ray Powder Diffraction (XRPD) Analysis of Example 1.

FIG. 2: Differential Scanning calorimetry (DSC) Analysis of Example 1.

FIG. 3: Thermogravimetric analysis (TGA) of Example 1.

FIG. 4: X-Ray Powder Diffraction (XRPD) Analysis of Example 2.

FIG. 5: Differential Scanning calorimetry (DSC) Analysis of Example 2.

FIG. 6: Thermogravimetric analysis (TGA) of Example 2.

### DETAILED DESCRIPTION OF THE INVENTION

### Processes

The inventors have identified a novel process for preparing compounds of formula (I). The compound of formula (I) may be represented chemically as (2S,3S,4S)-N-(5-chloro-2,4-difluorophenyl)-3,4-dihydroxy-N-(methyl-₃)-1-(6-methyl-4-(trifluoromethyppyridin-2-yl-5-oxopyrrolidine-2-carboxamide. The compound of formula (I) is disclosed in PCT/GB2020/051901 as a highly effective Polθ inhibitor for the treatment of cancer. Surprisingly, the novel process described herein (which comprises a number of innovative steps) prepares two differing crystalline polymorphic forms (referred to herein as Form A and Form B) which themselves are also claimed herein as novel pharmaceutical compounds as Polθ inhibitors for the treatment of cancer.

Thus, according to a first aspect of the invention, there is provided a process for preparing a compound of formula (I):

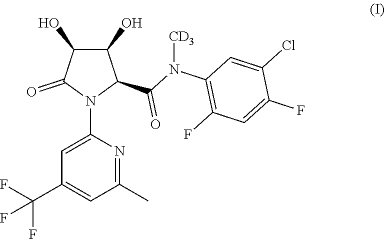 [SMILES: [2H]C([2H])([2H])N(C(=O)[C@@H]1[C@H](O)[C@H](O)C(=O)N1c1cc(C(F)(F)F)cc(C)n1)c1cc(Cl)c(F)cc1F]

which comprises treating a compound of formula (XX):

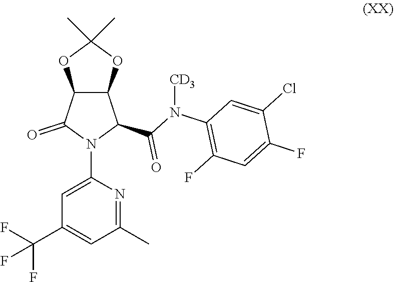 [SMILES: [2H]C([2H])([2H])N(C(=O)[C@@H]1[C@@H]2OC(C)(C)O[C@@H]2C(=O)N1c1cc(C(F)(F)F)cc(C)n1)c1cc(Cl)c(F)cc1F]

with a Lewis acid in the presence of a scavenger agent.

References herein to “scavenger agent” refer to any suitable agent which is capable of promoting the efficient cleavage of the isopropylidene ketal.

In one embodiment, the scavenger agent is a diol containing moiety. In a further embodiment, the diol containing moiety is selected from ethylene glycol, glycerol, 2,3-butanediol or meso-erythritol.

In a yet further embodiment, the diol containing moiety is meso-erythritol. It is believed that this is the first time that meso-erythritol has been used as an acetal deprotection scavenger agent.

In one embodiment, the Lewis acid is boron trifluoride (BF₃). In a further embodiment, the Lewis acid is boron trifluoride diethyl etherate.

According to a first aspect of the invention, there is provided a process for preparing a compound of formula (I):

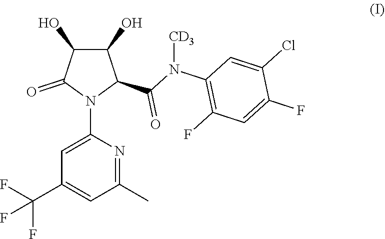 [SMILES: [2H]C([2H])([2H])N(C(=O)[C@@H]1[C@H](O)[C@H](O)C(=O)N1c1cc(C(F)(F)F)cc(C)n1)c1cc(Cl)c(F)cc1F]

which comprises the following steps:

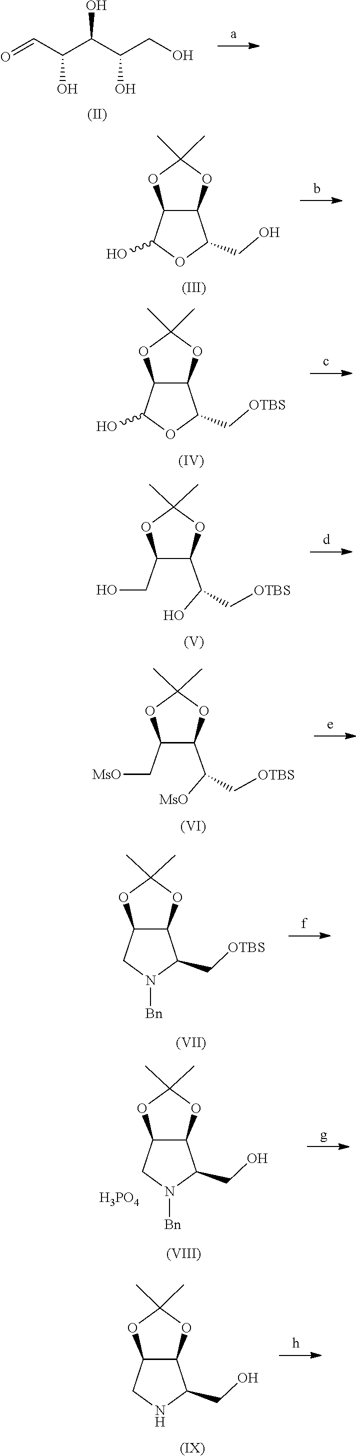

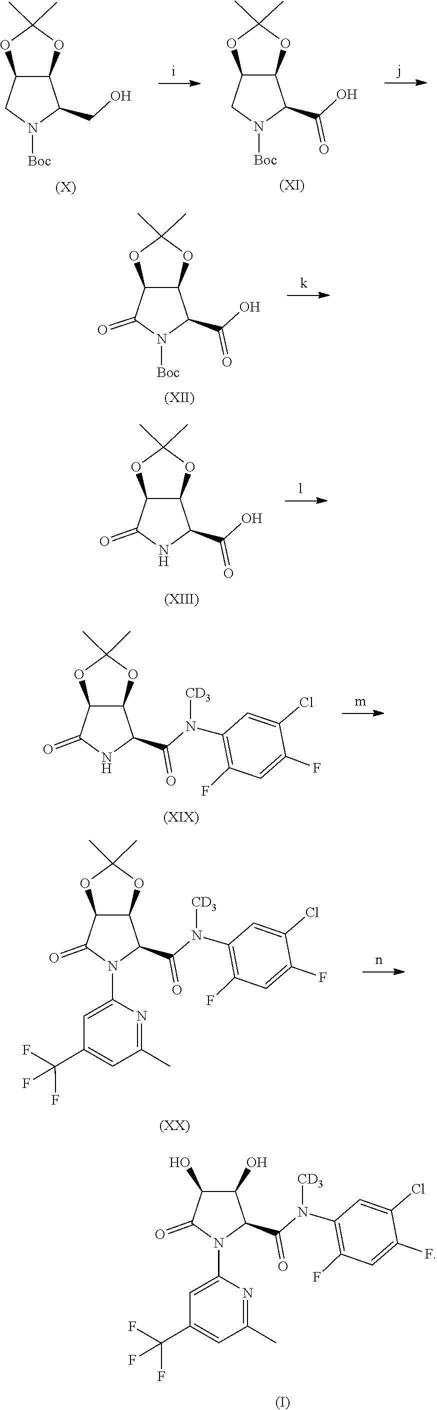

In a further embodiment, steps (a) to (k) may be conducted as described in Intermediate 1 and steps (l) to (n) may be conducted as described in Example 1. The process of this aspect of the invention surprisingly yielded a new crystalline polymorphic form of the compound of formula (I)—herein known as Form A (Example 1).

In an alternative aspect of the invention, there is provided a process for preparing a hemihydrate compound of formula (I), i.e. a compound of formula (IB):

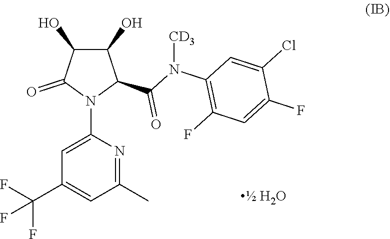 [SMILES: [2H]C([2H])([2H])N(C(=O)[C@@H]1[C@H](O)[C@H](O)C(=O)N1c1cc(C(F)(F)F)cc(C)n1)c1cc(Cl)c(F)cc1F]

which comprises the following steps:

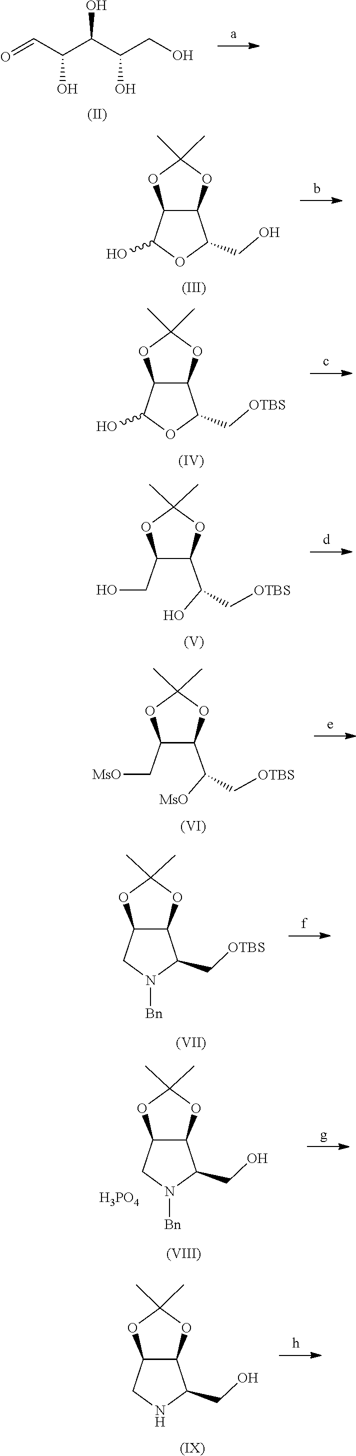

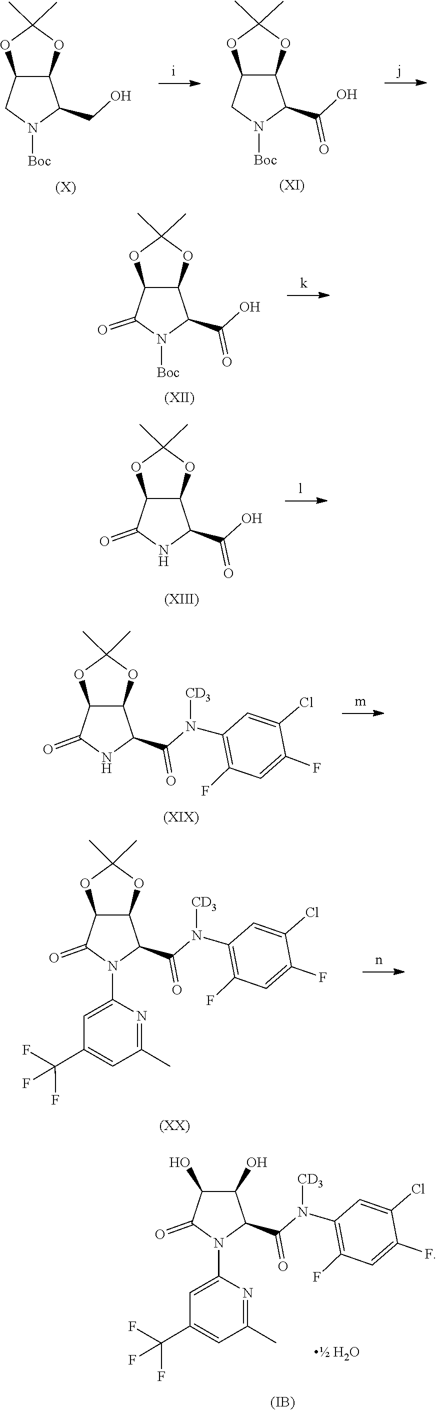

In a further embodiment, steps (a) to (k) may be conducted as described in Intermediate 1, steps (l) and (m) may be conducted as described in Example 1 and step (n) may be conducted as described in Example 2. The process of this aspect of the invention surprisingly yielded a new crystalline (hemihydrated) polymorphic form of the compound of formula (I)—herein known as Form B (Example 2).

According to a further aspect of the invention, there is provided a process for preparing a compound of formula (XX) as defined herein, which comprises reacting a compound of formula (XVIII):

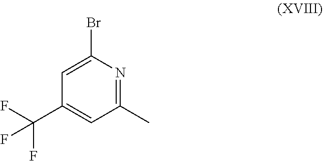

with a compound of formula (XIX):

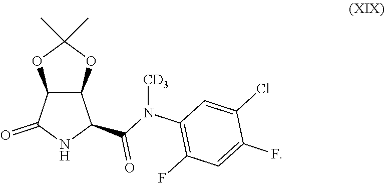 [SMILES: [2H]C([2H])([2H])N(C(=O)[C@H]1NC(=O)[C@H]2OC(C)(C)O[C@@H]12)c1cc(Cl)c(F)cc1F]

In one embodiment, the reaction typically comprises the use of a suitable catalyst, such as a copper catalyst, in particular copper (I) iodide, and a suitable ligand, such as N,N′-dimethylethylenediamine.

According to a further aspect of the invention, there is provided a process for preparing a compound of formula (XVI):

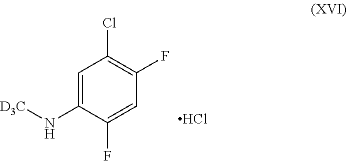 [SMILES: [2H]C([2H])([2H])Nc1cc(Cl)c(F)cc1F ; Cl]

which comprises reacting a compound of formula (XV):

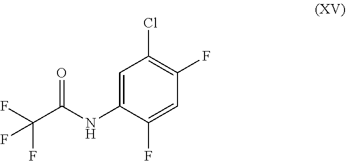 [SMILES: O=C(Nc1cc(Cl)c(F)cc1F)C(F)(F)F]

in a single vessel with methyl-d₃ iodide in the presence of an inorganic base, such as potassium carbonate, followed by treatment with potassium acetate and further addition of an inorganic base, such as potassium carbonate. In a further embodiment, the process additionally comprises isolation of the compound of formula (XVI) as a hydrochloride salt.

According to a further aspect of the invention, there is provided a process for preparing a compound of formula (XIII):

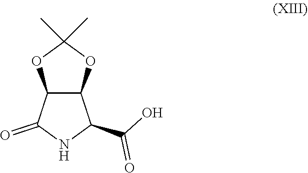

which comprises the use of a compound of formula (II):

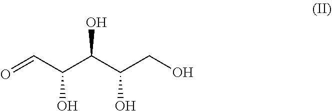

as the starting material.

In one embodiment, the process for preparing the compound of formula (XIII) comprises the following steps:

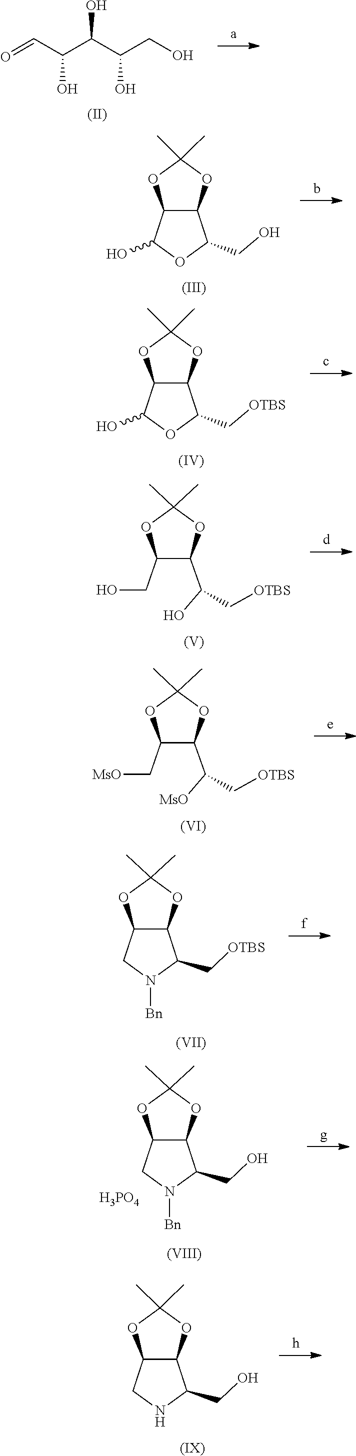

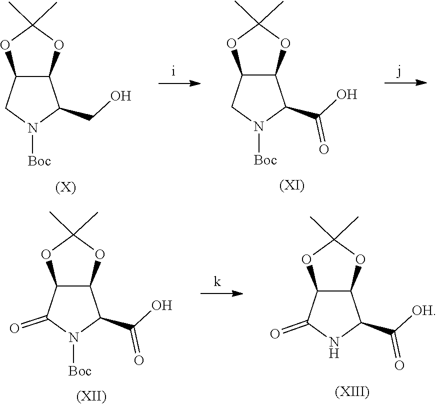

In a further embodiment, steps (a) to (k) may be conducted as described in Intermediate 1.

According to a further aspect of the invention, there is provided a process for preparing a compound of formula (XII) as defined herein, which comprises reacting a compound of formula (XI) as defined herein, with suitable oxidants, such as ruthenium dioxide and sodium periodate.

According to a further aspect of the invention, there is provided a process for preparing a compound of formula (XI) as defined herein, which comprises reacting a compound of formula (X) as defined herein, with suitable oxidants, such as ruthenium trichloride and sodium periodate.

According to a further aspect of the invention, there is provided a process for preparing a compound of formula (XII) as defined herein, which comprises reacting a compound of formula (X) as defined herein, with suitable oxidants, such as ruthenium trichloride and sodium periodate.

According to a further aspect of the invention, there is provided a process for preparing a compound of formula (VIII) as defined herein, which comprises reacting a compound of formula (VII) as defined herein, with a suitable acid, such as phosphoric acid. This process provides the advantage of isolating the product as a solid.

According to a further aspect of the invention, there is provided a process for preparing a compound of formula (V) as defined herein, which comprises the use of a compound of formula (II) as defined herein, as the starting material.

In one embodiment, the process for preparing the compound of formula (V) comprises the following steps:

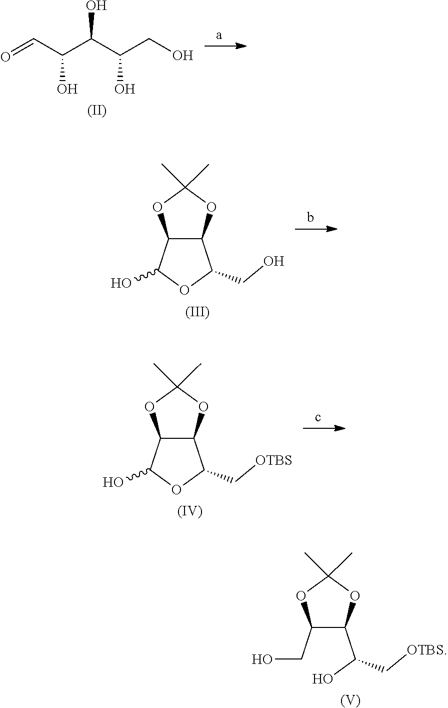

In a further embodiment, steps (a) to (c) may be conducted as described in Intermediate 1.

### Compounds of Formula (I)

As mentioned hereinbefore, the novel process of the invention results in the preparation of novel forms of the compound of formula (I) which themselves form an additional aspect of the invention.

Thus, according to a further aspect of the invention, there is provided a compound of formula (I) obtainable from the process as defined herein.

In one embodiment, the compound of formula (I) obtainable from the process as defined herein is (2S,3S,4S)-N-(5-Chloro-2,4-difluorophenyl)-3,4-dihydroxy-N-(methyl-d3)-1-(6-methyl-4-(trifluoromethyl)pyridin-2-yl)-5-oxopyrrolidine-2-carboxamide (Form A) (Example 1).

A person skilled in the art can determine by means of standard and long used techniques whether a polymorphic form has formed by the isolation conditions or purification conditions used to prepare a given compound. Examples of such techniques include thermogravimetric analysis (TGA), differential scanning calorimetry (DSC), X-ray crystallography (e.g. single crystal X-ray crystallography or X-ray powder diffraction) and Solid State NMR (SS-NMR, also known as Magic Angle Spinning NMR or MAS-NMR). Such techniques are as much a part of the standard analytical toolkit of the skilled chemist as NMR, IR, HPLC and MS.

A compound's X-ray powder pattern is characterised by the diffraction angle (2θ) and interplanar spacing (d) parameters of an X-ray diffraction spectrum. These are related by Bragg's equation, nλ=2d Sin θ, (where n=1; λ=wavelength of the cathode used; d=interplanar spacing; and θ=diffraction angle). Herein, interplanar spacings, diffraction angle and overall pattern are important for identification of crystal in the X-ray powder diffraction, due to the characteristics of the data. The relative intensity should not be strictly interpreted since it may be varied depending on the direction of crystal growth, particle sizes and measurement conditions. In addition, the diffraction angles usually mean ones which coincide in the range of 2θ±0.2°. The peaks mean main peaks and include peaks not larger than medium at diffraction angles other than those stated above.

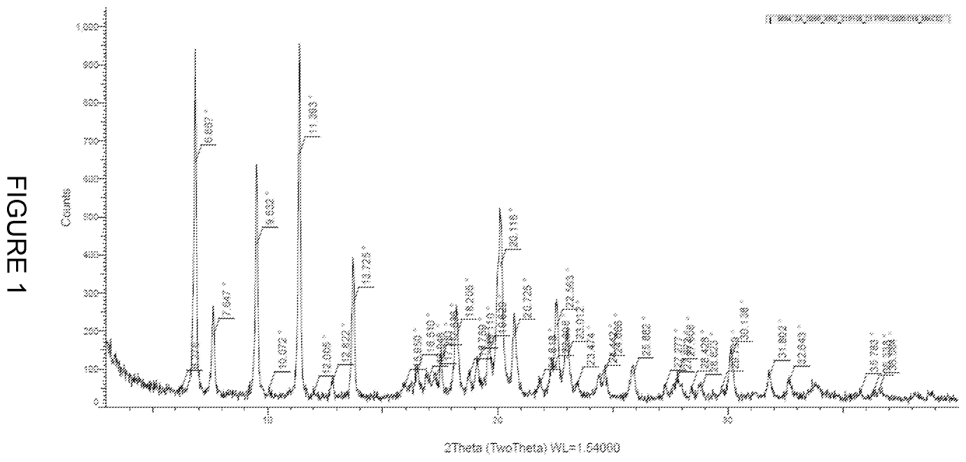

In a further embodiment, the Form A polymorph of (2S,3S,4S)-N-(5-Chloro-2,4-difluorophenyl)-3,4-dihydroxy-N-(methyl-d3)-1-(6-methyl-4-(trifluoromethyl)pyridin-2-yl)-5-oxopyrrolidine-2-carboxamide is characterised by an XRPD pattern substantially as shown in FIG. 1.

In a yet further embodiment, the Form A polymorph of (2S,3S,4S)-N-(5-Chloro-2,4-difluorophenyl)-3,4-dihydroxy-N-(methyl-d3)-1-(6-methyl-4-(trifluoromethyl)pyridin-2-yl)-5-oxopyrrolidine-2-carboxamide is characterised by having peaks at the same diffraction angles (2θ) of the XRPD pattern shown in FIG. 1 and optionally wherein the peaks have the same relative intensity as the peaks shown in FIG. 1.

It will be appreciated by the skilled person that references herein to “intensity” of peaks with respect to XRPD refer to relative intensities which have taken into account normalisation of background noise and other such parameters.

In a yet further embodiment, the Form A polymorph of (2S,3S,4S)-N-(5-Chloro-2,4-difluorophenyl)-3,4-dihydroxy-N-(methyl-d3)-1-(6-methyl-4-(trifluoromethyl)pyridin-2-yl)-5-oxopyrrolidine-2-carboxamide is characterised by having major peaks at diffraction angles (2θ) and intensities as those shown in the XRPD pattern in FIG. 1.

In a yet further embodiment, the Form A polymorph of (2S,3S,4S)-N-(5-Chloro-2,4-difluorophenyl)-3,4-dihydroxy-N-(methyl-d3)-1-(6-methyl-4-(trifluoromethyl)pyridin-2-yl)-5-oxopyrrolidine-2-carboxamide is characterised by an XRPD pattern having peaks at 6.9±0.5°, 7.6±0.5°, 9.5±0.5°, 11.4±0.5°, 13.7±0.5°, 20.1±0.5°, 20.7±0.5° and 22.6±0.5° (2θ, 1 d.p)

In a yet further embodiment, the Form A polymorph of (2S,3S,4S)-N-(5-Chloro-2,4-difluorophenyl)-3,4-dihydroxy-N-(methyl-d3)-1-(6-methyl-4-(trifluoromethyl)pyridin-2-yl)-5-oxopyrrolidine-2-carboxamide is characterised by an XRPD pattern having peaks at 6.9±0.2°, 7.6±0.2°, 9.5±0.2°, 11.4±0.2°, 13.7±0.2°, 20.1±0.2°, 20.7±0.2° and 22.6±0.2° (2θ, 1 d.p).

In a yet further embodiment, the Form A polymorph of (2S,3S,4S)-N-(5-Chloro-2,4-difluorophenyl)-3,4-dihydroxy-N-(methyl-d3)-1-(6-methyl-4-(trifluoromethyl)pyridin-2-yl)-5-oxopyrrolidine-2-carboxamide is characterised by an XRPD pattern having peaks at 6.9±0.1°, 7.6±0.1°, 9.5±0.1°, 11.4±0.1°, 13.7±0.1°, 20.1±0.1°, 20.7±0.1° and 22.6±0.1° (2θ, 1 d.p).

In a still yet further embodiment, the Form A polymorph of (2S,3S,4S)-N-(5-Chloro-2,4-difluorophenyl)-3,4-dihydroxy-N-(methyl-d3)-1-(6-methyl-4-(trifluoromethyl)pyridin-2-yl)-5-oxopyrrolidine-2-carboxamide is characterised by an XRPD pattern having peaks at 6.9, 7.6, 9.5, 11.4, 13.7, 20.1, 20.7 and 22.6 (20, 1d.p).

In a still yet further embodiment, the Form A polymorph of (2S,3S,4S)-N-(5-Chloro-2,4-difluorophenyl)-3,4-dihydroxy-N-(methyl-d3)-1-(6-methyl-4-(trifluoromethyl)pyridin-2-yl)-5-oxopyrrolidine-2-carboxamide is characterised by an XRPD pattern having peaks as set out in the below table:

| | Relative intensity, |
|---|---|
| Angle, °2θ | %* |
| 6.9 | 94.7 |
| 7.6 | 24 |
| 9.5 | 62.5 |
| 11.4 | 100 |
| 13.7 | 38 |
| 20.1 | 51.4 |
| 20.7 | 23.1 |
| 22.6 | 27.4 |

*Peaks with relative intensity of less than 20% are not reported.

In a further embodiment, the Form A polymorph of (2S,3S,4S)-N-(5-Chloro-2,4-difluorophenyl)-3,4-dihydroxy-N-(methyl-d3)-1-(6-methyl-4-(trifluoromethyl)pyridin-2-yl)-5-oxopyrrolidine-2-carboxamide is characterised by a differential scanning calorimetry (DSC) onset temperature of 182.26° C.±0.5° C. (such as 182.26° C.±0.2° C., in particular 182.26° C.±0.1° C., more particularly 182.26° C.).

In a further embodiment, the Form A polymorph of (2S,3S,4S)-N-(5-Chloro-2,4-difluorophenyl)-3,4-dihydroxy-N-(methyl-d3)-1-(6-methyl-4-(trifluoromethyl)pyridin-2-yl)-5-oxopyrrolidine-2-carboxamide is characterised by a differential scanning calorimetry (DSC) peak temperature of 182.54° C.±0.5° C. (such as 182.54° C.±0.2° C., in particular 182.54° C.±0.1° C., more particularly 182.54° C.).

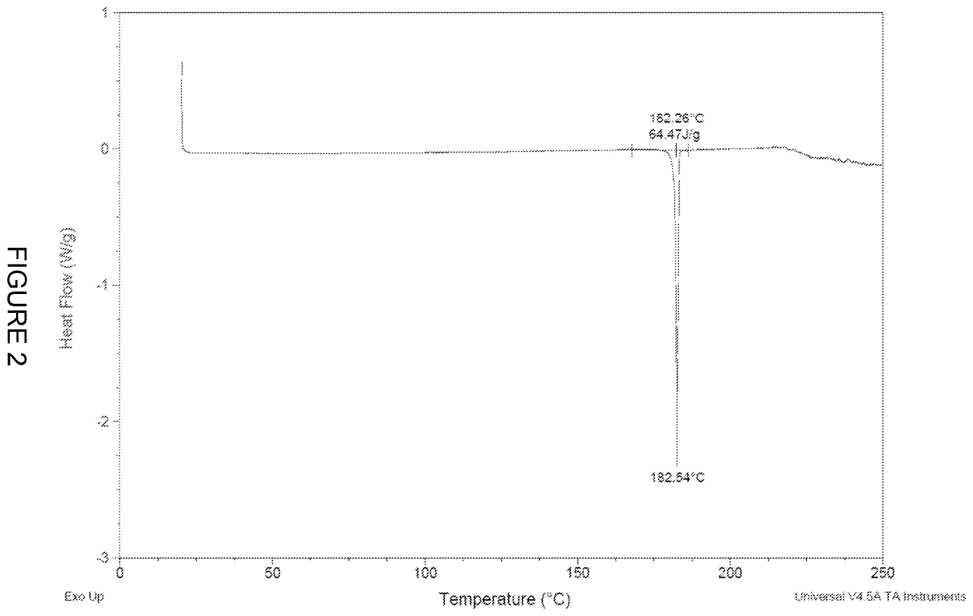

In a yet further embodiment, the Form A polymorph of (2S,3S,4S)-N-(5-Chloro-2,4-difluorophenyl)-3,4-dihydroxy-N-(methyl-d3)-1-(6-methyl-4-(trifluoromethyl)pyridin-2-yl)-5-oxopyrrolidine-2-carboxamide is characterised by a differential scanning calorimetry (DSC) thermogram as depicted in FIG. 2.

In a further embodiment, the Form A polymorph of (2S,3S,4S)-N-(5-Chloro-2,4-difluorophenyl)-3,4-dihydroxy-N-(methyl-d3)-1-(6-methyl-4-(trifluoromethyl)pyridin-2-yl)-5-oxopyrrolidine-2-carboxamide is characterised by a thermogravimetric peak mass loss at a temperature of 231.7° C.±0.5° C. (such as 231.7° C.±0.2° C., in particular 231.7° C.±0.1° C., more particularly 231.7° C.).

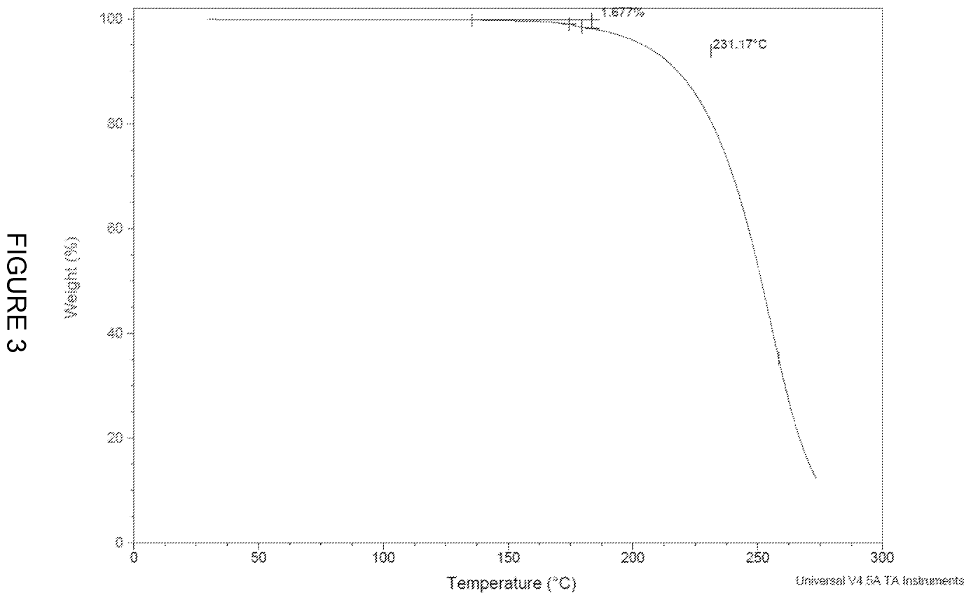

In a yet further embodiment, the Form A polymorph of (2S,3S,4S)-N-(5-Chloro-2,4-difluorophenyl)-3,4-dihydroxy-N-(methyl-d3)-1-(6-methyl-4-(trifluoromethyl)pyridin-2-yl)-5-oxopyrrolidine-2-carboxamide is characterised by a thermogravimetric analysis (TGA) thermogram as depicted in FIG. 3.

In an alternative embodiment, the compound of formula (I) obtainable from the process as defined herein is (2S,3S,4S)-N-(5-Chloro-2,4-difluorophenyI)-3,4-dihydroxy-N-(methyl-d3)-1-(6-methyl-4-(trifluoromethyl)pyridin-2-yl)-5-oxopyrrolidine-2-carboxamide hemihydrate (Form B) (Example 2).

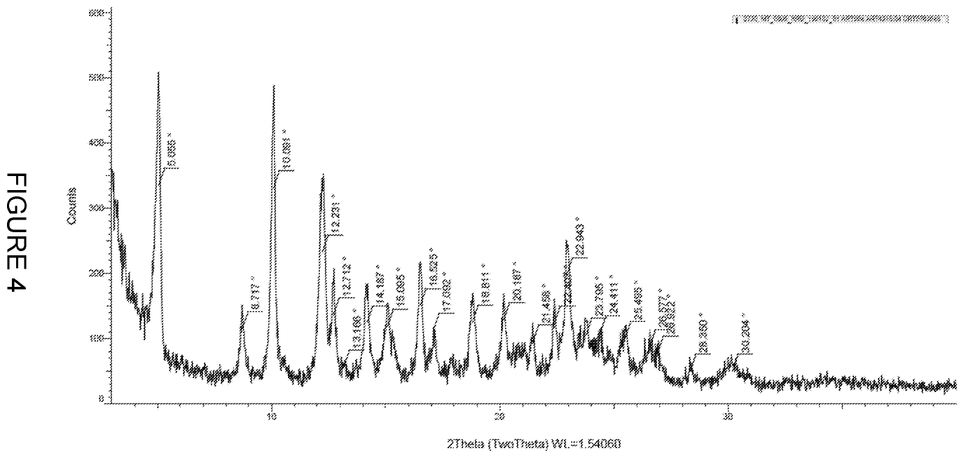

In a further embodiment, the Form B polymorph of (2S,3S,4S)-N-(5-Chloro-2,4-difluorophenyl)-3,4-dihydroxy-N-(methyl-d3)-1-(6-methyl-4-(trifluoromethyl)pyridin-2-yl)-5-oxopyrrolidine-2-carboxamide is characterised by an XRPD pattern substantially as shown in FIG. 4.

In a yet further embodiment, the Form B polymorph of (2S,3S,4S)-N-(5-Chloro-2,4-difluorophenyl)-3,4-dihydroxy-N-(methyl-d3)-1-(6-methyl-4-(trifluoromethyl)pyridin-2-yl)-5-oxopyrrolidine-2-carboxamide is characterised by having peaks at the same diffraction angles (2θ) of the XRPD pattern shown in FIG. 4 and optionally wherein the peaks have the same relative intensity as the peaks shown in FIG. 4.

In a yet further embodiment, the Form B polymorph of (2S,3S,4S)-N-(5-Chloro-2,4-difluorophenyl)-3,4-dihydroxy-N-(methyl-d3)-1-(6-methyl-4-(trifluoromethyl)pyridin-2-yl)-5-oxopyrrolidine-2-carboxamide is characterised by having major peaks at diffraction angles (2θ) and intensities as those shown in the XRPD pattern in FIG. 4.

In a yet further embodiment, the Form B polymorph of (2S,3S,4S)-N-(5-Chloro-2,4-difluorophenyl)-3,4-dihydroxy-N-(methyl-d3)-1-(6-methyl-4-(trifluoromethyl)pyridin-2-yl)-5-oxopyrrolidine-2-carboxamide is characterised by an XRPD pattern having peaks at 5.1±0.5°, 8.7±0.5°, 10.1±0.5°, 12.2±0.5°, 12.7±0.5°, 14.2±0.5°, 15.1±0.5°, 16.5±0.5°, 17.1±0.5°, 18.8±0.5°, 20.2±0.5°, 22.4±0.5° and 22.9±0.5° (2θ, 1 d.p).

In a yet further embodiment, the Form B polymorph of (2S,3S,4S)-N-(5-Chloro-2,4-difluorophenyl)-3,4-dihydroxy-N-(methyl-d3)-1-(6-methyl-4-(trifluoromethyl)pyridin-2-yl)-5-oxopyrrolidine-2-carboxamide is characterised by an XRPD pattern having peaks at 5.1±0.2°, 8.7±0.2°, 10.1±0.2°, 12.2±0.2°, 12.7±0.2°, 14.2±0.2°, 15.1±0.2°, 16.5±0.2°, 17.1±0.2°, 18.8±0.2°, 20.2±0.2°, 22.4±0.2° and 22.9±0.2° (2θ, 1 d.p).

In a yet further embodiment, the Form B polymorph of (2S,3S,4S)-N-(5-Chloro-2,4-difluorophenyl)-3,4-dihydroxy-N-(methyl-d3)-1-(6-methyl-4-(trifluoromethyl)pyridin-2-yl)-5-oxopyrrolidine-2-carboxamide is characterised by an XRPD pattern having peaks at 5.1±0.1°, 8.7±0.1°, 10.1±0.1°, 12.2±0.1°, 12.7±0.1°, 14.2±0.1°, 15.1±0.1°, 16.5±0.1°, 17.1±0.1°, 18.8±0.1°, 20.2±0.1°, 22.4±0.1° and 22.9±0.1° (2θ, 1 d.p).

In a still yet further embodiment, the Form B polymorph of (2S,3S,4S)-N-(5-Chloro-2,4-difluorophenyl)-3,4-dihydroxy-N-(methyl-d3)-1-(6-methyl-4-(trifluoromethyl)pyridin-2-yl)-5-oxopyrrolidine-2-carboxamide is characterised by an XRPD pattern having peaks at 5.1, 8.7, 10.1, 12.2, 12.7, 14.2, 15.1, 16.5, 17.1, 18.8, 20.2, 22.4 and 22.9 (2θ, 1 d.p).

In a still yet further embodiment, the Form B polymorph of (2S,3S,4S)-N-(5-Chloro-2,4-difluorophenyl)-3,4-dihydroxy-N-(methyl-d3)-1-(6-methyl-4-(trifluoromethyl)pyridin-2-yl)-5-oxopyrrolidine-2-carboxamide is characterised by an XRPD pattern having peaks as set out in the below table:

| | Relative intensity, |
|---|---|
| Angle, °2θ | %* |
| 5.1 | 84.4 |
| 8.7 | 24.7 |
| 10.1 | 100.0 |
| 12.2 | 66.4 |
| 12.7 | 32.3 |
| 14.2 | 27.0 |
| 15.1 | 25.8 |
| 16.5 | 36.4 |
| 17.1 | 22.0 |
| 18.8 | 26.7 |
| 20.2 | 28.0 |
| 22.4 | 24.3 |
| 22.9 | 48.7 |

*Peaks with relative intensity of less than 20% are not reported.

In a further embodiment, the Form B polymorph of (2S,3S,4S)-N-(5-Chloro-2,4-difluorophenyl)-3,4-dihydroxy-N-(methyl-d3)-1-(6-methyl-4-(trifluoromethyl)pyridin-2-yl)-5-oxopyrrolidine-2-carboxamide is characterised by a differential scanning calorimetry (DSC) onset temperature of 80.25° C.±0.5° C. (such as 80.25° C.±0.2° C., in particular 80.25° C.±0.1° C., more particularly 80.25° C.).

In a further embodiment, the Form B polymorph of (2S,3S,4S)-N-(5-Chloro-2,4-difluorophenyl)-3,4-dihydroxy-N-(methyl-d3)-1-(6-methyl-4-(trifluoromethyl)pyridin-2-yl)-5-oxopyrrolidine-2-carboxamide is characterised by a differential scanning calorimetry (DSC) peak temperature of 95.02° C.±0.5° C. (such as 95.02° C.±0.2° C., in particular 95.02° C.±0.1° C., more particularly 95.02° C.).

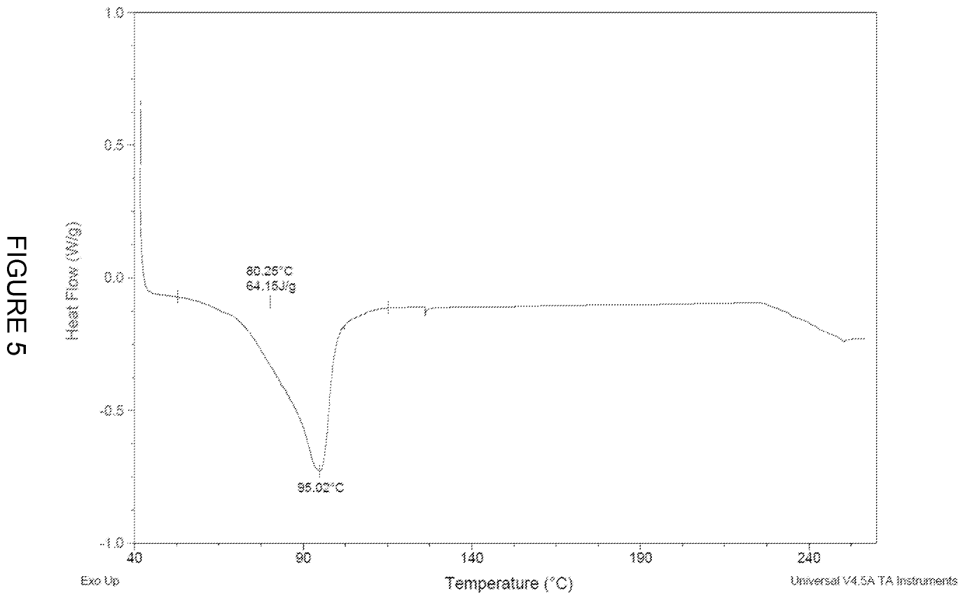

In a yet further embodiment, the Form B polymorph of (2S,3S,4S)-N-(5-Chloro-2,4-difluorophenyl)-3,4-dihydroxy-N-(methyl-d3)-1-(6-methyl-4-(trifluoromethyl)pyridin-2-yl)-5-oxopyrrolidine-2-carboxamide is characterised by a differential scanning calorimetry (DSC) thermogram as depicted in FIG. 5.

In a further embodiment, the Form B polymorph of (2S,3S,4S)-N-(5-Chloro-2,4-difluorophenyl)-3,4-dihydroxy-N-(methyl-d3)-1-(6-methyl-4-(trifluoromethyl)pyridin-2-yl)-5-oxopyrrolidine-2-carboxamide is characterised by a thermogravimetric peak mass loss at a temperature of 259.71° C.±0.5° C. (such as 259.71° C.±0.2° C., in particular 259.71° C.±0.1° C., more particularly 259.71° C.).

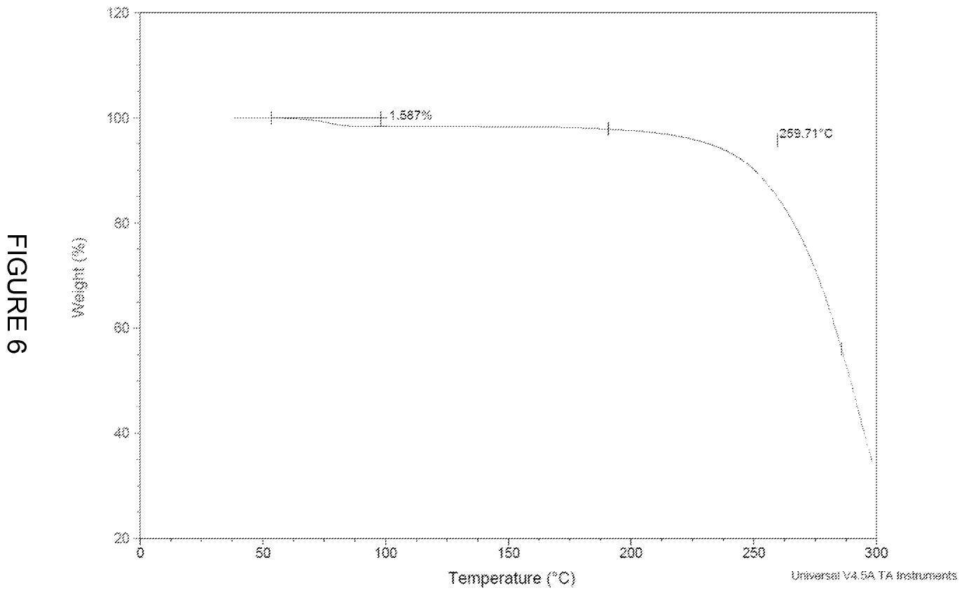

In a yet further embodiment, the Form B polymorph of (2S,3S,4S)-N-(5-Chloro-2,4-difluorophenyl)-3,4-dihydroxy-N-(methyl-d3)-1-(6-methyl-4-(trifluoromethyl)pyridin-2-yl)-5-oxopyrrolidine-2-carboxamide is characterised by a thermogravimetric analysis (TGA) thermogram as depicted in FIG. 6.

### Prodrugs

It will be appreciated by those skilled in the art that certain protected derivatives of compounds of formula (I), which may be made prior to a final deprotection stage, may not possess pharmacological activity as such, but may, in certain instances, be administered orally or parenterally and thereafter metabolised in the body to form compounds of the invention which are pharmacologically active. Such derivatives may therefore be described as “prodrugs”. All such prodrugs of compounds of the invention are included within the scope of the invention. Examples of pro-drug functionality suitable for the compounds of the present invention are described in Drugs of Today, 19, 9, 1983, 499-538 and in Topics in Chemistry, Chapter 31, pp. 306-316 and in “Design of Prodrugs” by H. Bundgaard, Elsevier, 1985, Chapter 1 (the disclosures in which documents are incorporated herein by reference). It will further be appreciated by those skilled in the art, that certain moieties, known to those skilled in the art as “pro-moieties”, for example as described by H. Bundgaard in “Design of Prodrugs” (the disclosure in which document is incorporated herein by reference) may be placed on appropriate functionalities when such functionalities are present within compounds of the invention.

Also included within the scope of the compounds of the invention are further polymorphs thereof.

### Enantiomers

Where chiral centres are present in compounds of formula (I), the present invention includes within its scope all possible enantiomers and diastereoisomers, including mixtures thereof. The different isomeric forms may be separated or resolved one from the other by conventional methods, or any given isomer may be obtained by conventional synthetic methods or by stereospecific or asymmetric syntheses. The invention also extends to any tautomeric forms or mixtures thereof.

### Isotopes

The subject invention also includes all pharmaceutically acceptable isotopically-labelled compounds which are identical to those recited in formula (I) but for the fact that one or more atoms are replaced by an atom having an atomic mass or mass number different from the atomic mass or mass number most commonly found in nature.

Examples of isotopes suitable for inclusion in the compounds of the invention comprise isotopes of hydrogen, such as ²H (D) and ³H (T), carbon, such as ¹¹C, ¹³C and ¹⁴C, chlorine, such as ³⁶Cl, fluorine, such as ¹⁸F, iodine, such as ¹²³I, ¹²⁵I and ¹³¹I, nitrogen, such as ¹³N and ¹⁵N, oxygen, such as ¹⁵O, ¹⁷O and ¹⁸O, phosphorus, such as ³²P, and sulfur, such as ³⁵S.

Certain isotopically-labelled compounds of formula (I), for example, those incorporating a radioactive isotope, are useful in drug and/or substrate tissue distribution studies. The compounds of formula (I) can also have valuable diagnostic properties in that they can be used for detecting or identifying the formation of a complex between a labelled compound and other molecules, peptides, proteins, enzymes or receptors. The detecting or identifying methods can use compounds that are labelled with labelling agents such as radioisotopes, enzymes, fluorescent substances, luminous substances (for example, luminol, luminol derivatives, luciferin, aequorin and luciferase) etc. The radioactive isotopes tritium, i.e. ³H (T), and carbon-14, i.e. ¹⁴C, are particularly useful for this purpose in view of their ease of incorporation and ready means of detection.

Substitution with heavier isotopes such as deuterium, i.e. ²H (D), may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining target occupancy.

Isotopically-labelled compounds of formula (I) can generally be prepared by conventional techniques known to those skilled in the art or by processes analogous to those described in the accompanying Examples and Preparations using appropriate isotopically-labelled reagents in place of the non-labelled reagent previously employed.

### Purity

Since the compounds of formula (I) are intended for use in pharmaceutical compositions it will readily be understood that they are each preferably provided in substantially pure form, for example at least 60% pure, more suitably at least 75% pure and preferably at least 85%, especially at least 98% pure (% are given on a weight for weight basis). Impure preparations of the compounds may be used for preparing the more pure forms used in the pharmaceutical compositions.

Therapeutic Utility

The compounds of the invention, subgroups and examples thereof, are inhibitors of Polθ polymerase activity, and which may be useful in preventing or treating disease states or conditions described herein. In addition, the compounds of the invention, and subgroups thereof, will be useful in preventing or treating diseases or condition mediated by Polθ. References to the preventing or prophylaxis or treatment of a disease state or condition such as cancer include within their scope alleviating or reducing the incidence of cancer.

Thus, for example, it is envisaged that the compounds of the invention will be useful in alleviating or reducing the incidence of cancer.

The compounds of the present invention may be useful for the treatment of the adult population. The compounds of the present invention may be useful for the treatment of the pediatric population.

As a consequence of their inhibition of Polθ, the compounds will be useful in providing a means of disabling the ability of cells to perform MMEJ. It is therefore anticipated that the compounds may prove useful in treating or preventing proliferative disorders such as cancers. In addition, the compounds of the invention may be useful in the treatment of diseases in which there is a disorder associated with cell accumulation.

Without being bound by theory it is expected that the Polθ inhibitors of the present invention will demonstrate certain properties for them to be of particular utility in the therapeutic treatment of certain cancers. For example, in one embodiment, the Polθ inhibitors of the present invention are suitably lethal in BRCA1 and BRCA2 deficient primary and secondary solid tumours, including breast, ovarian, prostate and pancreas.

In a further embodiment, the Polθ inhibitors of the present invention are suitably lethal in a variety of primary and secondary solid tumours which are HRD by mechanisms other than BRCA deficiency, such as those with promoter hypermethylation. In these tumours where no DSB repair pathway may be fully down regulated the Polei may be given along with another DDR modulator such as a PARP inhibitor, a DNA-PK inhibitor, an ATR inhibitor, an ATM inhibitor, a weel inhibitor or a CHK1 inhibitor.

In a further embodiment, the Polθ inhibitors of the present invention are suitably lethal in primary and secondary breast, ovarian, prostate and pancreatic tumours retaining BRCA1 deficiency but which, following or not following exposure to PARPi medication, are resistant to PARPi treatment.

In a further embodiment, the Polθ inhibitors of the present invention suitably increase the ORR including CRR, will delay the onset of PARPi resistance, will increase the time to relapse and DFS, and will increase the OS of HRD (BRCA1/2 deficient and other HRD mechanisms) primary and secondary tumours (breast, ovarian, prostate and pancreas) when given with PARPi treatment programmes.

In a further embodiment, the Polθ inhibitors of the present invention suitably show synthetic sickness and/or synthetic lethality in a variety of tumours with loss of ATM activity (ATM^{-/-}) particularly in the context of VVT p53. Tumour types will include around 10% of all solid tumours including gastric, lung, breast, and CRC, along with CLL. Co-medicating with another DDR modifier, such as a DNA-PK inhibitor, PARP inhibitor or ATR inhibitor, may further enhance such activity. Polθ inhibitors will resensitise CLL to classical chemotherapy and chemo-immunotherapy where drug resistance has emerged. Thus, according to a further embodiment, the pharmaceutical composition of the present invention additionally comprises a DNA-PK inhibitor, PARP inhibitor or ATR inhibitor.

In a further embodiment, the Polθ inhibitors of the present invention suitably show synthetic sickness and/or synthetic lethality in a variety of tumours deficient in the DNA double strand break repair process of non-homologous end-joining (NHEJ-D). Tumour types will include approximately 2-10% of all solid tumours including prostate, pancreatic, cervical, breast, lung, bladder and oesophageal. Co-medicating with another DDR modifier, such as a PARP inhibitor, ATM inhibitor, weel inhibitor, CHK inhibitor, or ATR inhibitor, may further enhance such activity. Polθ inhibitors will further sensitise NHEJD cancer cells to DNA DSB inducing chemotherapies and to ionising radiation based therapies. Thus, according to a further embodiment, the pharmaceutical composition of the present invention additionally comprises a PARP inhibitor, ATM inhibitor, wee1 inhibitor, CHK inhibitor, or ATR inhibitor.

In a further embodiment, the Polθ inhibitors of the present invention suitably reduce the DNA replication stress response during the chemotherapy of HR proficient tumours such as ovarian, NSCL and breast tumours over expressing Polθ. This will increase the ORR to treatment and increase OS. Such effects are particularly likely with cytarabine (Ara-C) and hydroxyurea used in a wide variety of leukemias including CML, and the management of squamous cell carcinomas.

In a further embodiment, the Polθ inhibitors of the present invention suitably selectively sensitise solid tumours to radiotherapy, including EBRT and brachytherapy and radioligand based therapies, with little or no sensitisation of normal tissues. In a fractionated curative-intent setting this will increase loco-regional control driving increased survival. This will be particularly evident in the management of NSCLC, SCCH&N, rectal cancer, prostate cancer and pancreatic cancer.

In a further embodiment, the Polθ inhibitors of the present invention suitably show synthetic sickness and/or synthetic lethality in PTEN deleted tumours such as CaP, with or without comedication with a PARPi. Furthermore, such tumours will exhibit exquisite sensitivity to radiotherapy both by dint of the PTEN deletion as well as the Polθ inhibitor induced radiosensitivity.

In a further embodiment, the Polθ inhibitors of the present invention suitably suppress TLS polymerase activity, sensitising primary and secondary solid tumours (e.g. breast, lung, ovarian, CRC) to drugs (e.g. cisplatin, mitomycin and cyclophosphamide) as well as reducing the acquisition of drug-induced mutations implicated in tumour resistance leading to prolongation of remission and increased TTR.

In a further embodiment, the Polθ inhibitors of the present invention suitably resensitise BCR-ABL-positive CML which is has developed imatinib resistance, as well as other solid tumours with elevated ligase IIIα levels, reduced ligase IV levels and increased dependence upon altEJ DSB repair.

In a further embodiment, the Polθ inhibitors of the present invention suitably show synthetic sickness and/or synthetic lethality in aromatase inhibitor resistant ER⁻ primary and secondary breast cancers, again showing elevated ligase IIla levels, reduced ligase IV levels and increased dependence upon altEJ DSB repair.

According to a further aspect of the invention there is a provided a compound of formula (I) as defined herein for use in the treatment of tumours characterised by a deficiency in homologous recombination (HRD).

It will be appreciated that references herein to “deficiency in homologous recombination (HRD)” refer to any genetic variation which results in a deficiency or loss of function of the resultant homologous recombination gene. Examples of said genetic variation include mutations (e.g. point mutations), substitutions, deletions, single nucleotide polymorphisms (SNPs), haplotypes, chromosome abnormalities, Copy Number Variation (CNV), epigenetics, DNA inversions, reduction in expression and mis-localisation.

In one embodiment, said homologous recombination genes are selected from any of: ATM, ATR, BRCA1, BRCA2, BARD1, RAD51C, RAD50, CHEK1, CHEK2, FANCA, FANCB, FANCC, FANCD2, FANCE, FANCF, FANCG, FANCI, FANCL, FANCM, PALB2 (FANCN), FANCP (BTBD12), ERCC4 (FANCQ), PTEN, CDK12, MRE11, NBS1, NBN, CLASPIN, BLM, WRN, SMARCA2, SMARCA4, LIG1, RPA1, RPA2, BRIP1 and PTEN.

It will be appreciated that references herein to “non-homologous end-joining deficiency (NHEJD)” refer to any genetic variation which results in a deficiency or loss of function of the resultant homologous recombination gene. Examples of said genetic variation include mutations (e.g. point mutations), substitutions, deletions, single nucleotide polymorphisms (SNPs), haplotypes, chromosome abnormalities, Copy Number Variation (CNV), epigenetics, DNA inversions, reduction in expression and mis-localisation.

In one embodiment, said non-homologous end-joining genes are selected from any one or more of: LIG4, NHEJ1, POLL, POLM, PRKDC, XRCC4, XRCC5, XRCC6, and DCLRE1C.

According to a further aspect of the invention there is a provided a compound of formula (I) as defined herein for use in the treatment of tumours which overexpress Polθ.

According to a further aspect of the invention there is a provided a compound of formula (I) as defined herein for use in the treatment of tumours which have elevated ligase IIIα levels, reduced ligase IV levels and increased dependence upon altEJ DSB repair.

Examples of cancers (and their benign counterparts) which may be treated (or inhibited) include, but are not limited to tumours of epithelial origin (adenomas and carcinomas of various types including adenocarcinomas, squamous carcinomas, transitional cell carcinomas and other carcinomas) such as carcinomas of the bladder and urinary tract, breast, gastrointestinal tract (including the esophagus, stomach (gastric), small intestine, colon, rectum and anus), liver (hepatocellular carcinoma), gall bladder and biliary system, exocrine pancreas, kidney, lung (for example adenocarcinomas, small cell lung carcinomas, non-small cell lung carcinomas, bronchioalveolar carcinomas and mesotheliomas), head and neck (for example cancers of the tongue, buccal cavity, larynx, pharynx, nasopharynx, tonsil, salivary glands, nasal cavity and paranasal sinuses), ovary, fallopian tubes, peritoneum, vagina, vulva, penis, cervix, myometrium, endometrium, thyroid (for example thyroid follicular carcinoma), adrenal, prostate, skin and adnexae (for example melanoma, basal cell carcinoma, squamous cell carcinoma, keratoacanthoma, dysplastic naevus); haematological malignancies (i.e. leukemias, lymphomas) and premalignant haematological disorders and disorders of borderline malignancy including haematological malignancies and related conditions of lymphoid lineage (for example acute lymphocytic leukemia [ALL], chronic lymphocytic leukemia [CLL], B-cell lymphomas such as diffuse large B-cell lymphoma [DLBCL], follicular lymphoma, Burkitt's lymphoma, mantle cell lymphoma, MALT lymphoma, T-cell lymphomas and leukaemias, natural killer [NK] cell lymphomas, Hodgkin's lymphomas, hairy cell leukaemia, monoclonal gammopathy of uncertain significance, plasmacytoma, multiple myeloma, and post-transplant lymphoproliferative disorders), and haematological malignancies and related conditions of myeloid lineage (for example acute myelogenous leukemia [AML], chronic myelogenous leukemia [CML], chronic myelomonocytic leukemia [CMML], hypereosinophilic syndrome, myeloproliferative disorders such as polycythaemia vera, essential thrombocythaemia and primary myelofibrosis, myeloproliferative syndrome, myelodysplastic syndrome, and promyelocytic leukemia); tumours of mesenchymal origin, for example sarcomas of soft tissue, bone or cartilage such as osteosarcomas, fibrosarcomas, chondrosarcomas, rhabdomyosarcomas, leiomyosarcomas, liposarcomas, angiosarcomas, Kaposi's sarcoma, Ewing's sarcoma, synovial sarcomas, epithelioid sarcomas, gastrointestinal stromal tumours, benign and malignant histiocytomas, and dermatofibrosarcoma protuberans; tumours of the central or peripheral nervous system (for example astrocytomas, gliomas and glioblastomas, meningiomas, ependymomas, pineal tumours and schwannomas); endocrine tumours (for example pituitary tumours, adrenal tumours, islet cell tumours, parathyroid tumours, carcinoid tumours and medullary carcinoma of the thyroid); ocular and adnexal tumours (for example retinoblastoma); germ cell and trophoblastic tumours (for example teratomas, seminomas, dysgerminomas, hydatidiform moles and choriocarcinomas); and paediatric and embryonal tumours (for example medulloblastoma, neuroblastoma, Wlms tumour, and primitive neuroectodermal tumours); or syndromes, congenital or otherwise, which leave the patient susceptible to malignancy (for example Xeroderma Pigmentosum).

Many diseases are characterized by persistent and unregulated angiogenesis. Chronic proliferative diseases are often accompanied by profound angiogenesis, which can contribute to or maintain an inflammatory and/or proliferative state, or which leads to tissue destruction through the invasive proliferation of blood vessels. Tumour growth and metastasis have been found to be angiogenesis-dependent. Compounds of the invention may therefore be useful in preventing and disrupting initiation of tumour angiogenesis. In particular, the compounds of the invention may be useful in the treatment of metastasis and metastatic cancers.

Metastasis or metastatic disease is the spread of a disease from one organ or part to another non-adjacent organ or part. The cancers which can be treated by the compounds of the invention include primary tumours (i.e. cancer cells at the originating site), local invasion (cancer cells which penetrate and infiltrate surrounding normal tissues in the local area), and metastatic (or secondary) tumours ie. tumours that have formed from malignant cells which have circulated through the bloodstream (haematogenous spread) or via lymphatics or across body cavities (trans-coelomic) to other sites and tissues in the body.

Particular cancers include hepatocellular carcinoma, melanoma, oesophageal, renal, colon, colorectal, lung e.g. mesothelioma or lung adenocarcinoma, breast, bladder, gastrointestinal, ovarian and prostate cancers.

A further aspect provides the use of a compound for the manufacture of a medicament for the treatment of a disease or condition as described herein, in particular cancer.

The compounds may also be useful in the treatment of tumour growth, pathogenesis, resistance to chemo- and radio-therapy by sensitising cells to chemotherapy and as an anti-metastatic agent.

The potency of the compounds of the invention as inhibitors of Pole can be measured using the biological and biophysical assays set forth in the examples herein and the level of affinity exhibited by a given compound can be defined in terms of the ICso value. Particular compounds of the present invention are compounds having an ICso value of less than 1 μM, more particularly less than 0.1 μM.

A role for the loss of Polθ enhancing the efficacy of CRISPR mediated gene editing has been described in WO 2017/062754.Thus, Polθ inhibitory compounds are likely to be useful in enhancing the efficiency of CRISPR based editing methodologies and/or CRISPR based editing therapeutics. Furthermore, compound mediated Polθ inhibition is likely to reduce the frequency of random integration events and thus provide a route to ameliorate any safety concerns of CRISPR mediated technology. Thus, according to a further aspect of the invention, there is provided the use of a compound of formula (I) as defined herein in a CRISPR based editing methodology and/or CRISPR based editing therapeutics, such as the enhancement of efficiency of CRISPR based editing methodology and/or CRISPR based editing therapeutics.

### Pharmaceutical Compositions

While it is possible for the active compound to be administered alone, it is preferable to present it as a pharmaceutical composition (e.g. formulation). In one embodiment this is a sterile pharmaceutical composition.

Thus, the present invention further provides pharmaceutical compositions, as defined above, and methods of making a pharmaceutical composition comprising (e.g admixing) at least one compound of formula (I) (and sub-groups thereof as defined herein), together with one or more pharmaceutically acceptable excipients and optionally other therapeutic or prophylactic agents, as described herein.

The pharmaceutically acceptable excipient(s) can be selected from, for example, carriers (e.g. a solid, liquid or semi-solid carrier), adjuvants, diluents, fillers or bulking agents, granulating agents, coating agents, release-controlling agents, binding agents, disintegrants, lubricating agents, preservatives, antioxidants, buffering agents, suspending agents, thickening agents, flavouring agents, sweeteners, taste masking agents, stabilisers or any excipients for various types of pharmaceutical compositions are set out in more detail below.

The term “pharmaceutically acceptable” as used herein pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Pharmaceutical compositions containing compounds of the formula (I) can be formulated in accordance with known techniques, see for example, Remington's Pharmaceutical Sciences, Mack Publishing Company, Easton, PA, USA.

The pharmaceutical compositions can be in any form suitable for oral, parenteral, topical, intranasal, intrabronchial, sublingual, ophthalmic, otic, rectal, intra-vaginal, or transdermal administration. Where the compositions are intended for parenteral administration, they can be formulated for intravenous, intramuscular, intraperitoneal, subcutaneous administration or for direct delivery into a target organ or tissue by injection, infusion or other means of delivery. The delivery can be by bolus injection, short term infusion or longer term infusion and can be via passive delivery or through the utilisation of a suitable infusion pump or syringe driver.

Pharmaceutical formulations adapted for parenteral administration include aqueous and non-aqueous sterile injection solutions which may contain anti-oxidants, buffers, bacteriostats, co-solvents, surface active agents, organic solvent mixtures, cyclodextrin complexation agents, emulsifying agents (for forming and stabilizing emulsion formulations), liposome components for forming liposomes, gellable polymers for forming polymeric gels, lyophilisation protectants and combinations of agents for, inter alia, stabilising the active ingredient in a soluble form and rendering the formulation isotonic with the blood of the intended recipient. Pharmaceutical formulations for parenteral administration may also take the form of aqueous and non-aqueous sterile suspensions which may include suspending agents and thickening agents (R. G. Strickly, Solubilizing Excipients in oral and injectable formulations, Pharmaceutical Research, Vol 21(2) 2004, p 201-230).

The formulations may be presented in unit-dose or multi-dose containers, for example sealed ampoules, vials and prefilled syringes, and may be stored in a freeze-dried (lyophilised) condition requiring only the addition of the sterile liquid carrier, for example water for injections, immediately prior to use. In one embodiment, the formulation is provided as an active pharmaceutical ingredient in a bottle for subsequent reconstitution using an appropriate diluent.

The pharmaceutical formulation can be prepared by lyophilising a compound of formula (I), or sub-groups thereof. Lyophilisation refers to the procedure of freeze-drying a composition. Freeze-drying and lyophilisation are therefore used herein as synonyms.

Extemporaneous injection solutions and suspensions may be prepared from sterile powders, granules and tablets.

Pharmaceutical compositions of the present invention for parenteral injection can also comprise pharmaceutically acceptable sterile aqueous or non-aqueous solutions, dispersions, suspensions or emulsions as well as sterile powders for reconstitution into sterile injectable solutions or dispersions just prior to use.

Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as sunflower oil, safflower oil, corn oil or olive oil), and injectable organic esters such as ethyl oleate. Proper fluidity can be maintained, for example, by the use of thickening or coating materials such as lecithin, by the maintenance of the required particle size in the case of dispersions, and by the use of surfactants.

The compositions of the present invention may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol, sorbic acid, and the like. It may also be desirable to include agents to adjust tonicity such as sugars, sodium chloride, and the like. Prolonged absorption of the injectable pharmaceutical form may be brought about by the inclusion of agents which delay absorption such as aluminum monostearate and gelatin.

In one particular embodiment of the invention, the pharmaceutical composition is in a form suitable for i.v. administration, for example by injection or infusion. For intravenous administration, the solution can be dosed as is, or can be injected into an infusion bag (containing a pharmaceutically acceptable excipient, such as 0.9% saline or 5% dextrose), before administration.

In another particular embodiment, the pharmaceutical composition is in a form suitable for sub-cutaneous (s.c.) administration.

Pharmaceutical dosage forms suitable for oral administration include tablets (coated or uncoated), capsules (hard or soft shell), caplets, pills, lozenges, syrups, solutions, powders, granules, elixirs and suspensions, sublingual tablets, wafers or patches such as buccal patches.

Thus, tablet compositions can contain a unit dosage of active compound together with an inert diluent or carrier such as a sugar or sugar alcohol, eg; lactose, sucrose, sorbitol or mannitol; and/or a non-sugar derived diluent such as sodium carbonate, calcium phosphate, calcium carbonate, or a cellulose or derivative thereof such as microcrystalline cellulose (MCC), methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Tablets may be designed to release the drug either upon contact with stomach fluids (immediate release tablets) or to release in a controlled manner (controlled release tablets) over a prolonged period of time or with a specific region of the GI tract.

Capsule formulations may be of the hard gelatin or soft gelatin variety and can contain the active component in solid, semi-solid, or liquid form. Gelatin capsules can be formed from animal gelatin or synthetic or plant derived equivalents thereof.

The solid dosage forms (eg; tablets, capsules etc.) can be coated or un-coated. Coatings may act either as a protective film (e.g. a polymer, wax or varnish) or as a mechanism for controlling drug release or for aesthetic or identification purposes. The coating (e.g. a Eudragit™ type polymer) can be designed to release the active component at a desired location within the gastro-intestinal tract. Thus, the coating can be selected so as to degrade under certain pH conditions within the gastrointestinal tract, thereby selectively release the compound in the stomach or in the ileum, duodenum, jejenum or colon.

Instead of, or in addition to, a coating, the drug can be presented in a solid matrix comprising a release controlling agent, for example a release delaying agent which may be adapted to release the compound in a controlled manner in the gastrointestinal tract. Alternatively the drug can be presented in a polymer coating e.g. a polymethacrylate polymer coating, which may be adapted to selectively release the compound under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract. In another alternative, the coating can be designed to disintegrate under microbial action in the gut. As a further alternative, the active compound can be formulated in a delivery system that provides osmotic control of the release of the compound. Osmotic release and other delayed release or sustained release formulations (for example formulations based on ion exchange resins) may be prepared in accordance with methods well known to those skilled in the art.

The compound of formula (I) may be formulated with a carrier and administered in the form of nanoparticles, the increased surface area of the nanoparticles assisting their absorption. In addition, nanoparticles offer the possibility of direct penetration into the cell. Nanoparticle drug delivery systems are described in “Nanoparticle Technology for Drug Delivery”, edited by Ram B Gupta and Uday B. Kompella, Informa Healthcare, ISBN 9781574448573, published 13 Mar. 2006. Nanoparticles for drug delivery are also described in J. Control. Release, 2003, 91 (1-2), 167-172, and in Sinha et al., Mol. Cancer Ther. Aug. 1, (2006) 5, 1909.

The pharmaceutical compositions typically comprise from approximately 1% (w/w) to approximately 95% (w/w) active ingredient and from 99% (w/w) to 5% (w/w) of a pharmaceutically acceptable excipient or combination of excipients. Particularly, the compositions comprise from approximately 20% (w/w) to approximately 90% (w/w) active ingredient and from 80% (w/w) to 10% of a pharmaceutically acceptable excipient or combination of excipients. The pharmaceutical compositions comprise from approximately 1% to approximately 95%, particularly from approximately 20% to approximately 90%, active ingredient. Pharmaceutical compositions according to the invention may be, for example, in unit dose form, such as in the form of ampoules, vials, suppositories, pre-filled syringes, dragées, tablets or capsules.

The pharmaceutically acceptable excipient(s) can be selected according to the desired physical form of the formulation and can, for example, be selected from diluents (e.g solid diluents such as fillers or bulking agents; and liquid diluents such as solvents and co-solvents), disintegrants, buffering agents, lubricants, flow aids, release controlling (e.g. release retarding or delaying polymers or waxes) agents, binders, granulating agents, pigments, plasticizers, antioxidants, preservatives, flavouring agents, taste masking agents, tonicity adjusting agents and coating agents.

The skilled person will have the expertise to select the appropriate amounts of ingredients for use in the formulations. For example, tablets and capsules typically contain 0-20% disintegrants, 0-5% lubricants, 0-5% flow aids and/or 0-99% (w/w) fillers/ or bulking agents (depending on drug dose). They may also contain 0-10% (w/w) polymer binders, 0-5% (w/w) antioxidants, 0-5% (w/w) pigments. Slow release tablets would in addition contain 0-99% (w/w) release-controlling (e.g. delaying) polymers (depending on dose). The film coats of the tablet or capsule typically contain 0-10% (w/w) polymers, 0-3% (w/w) pigments, and/or 0-2% (w/w) plasticizers.

Parenteral formulations typically contain 0-20% (w/w) buffers, 0-50% (w/w) cosolvents, and/or 0-99% (w/w) Water for Injection (WFI) (depending on dose and if freeze dried). Formulations for intramuscular depots may also contain 0-99% (w/w) oils.

Pharmaceutical compositions for oral administration can be obtained by combining the active ingredient with solid carriers, if desired granulating a resulting mixture, and processing the mixture, if desired or necessary, after the addition of appropriate excipients, into tablets, dragee cores or capsules. It is also possible for them to be incorporated into a polymer or waxy matrix that allow the active ingredients to diffuse or be released in measured amounts.

The compounds of the invention can also be formulated as solid dispersions. Solid dispersions are homogeneous extremely fine disperse phases of two or more solids. Solid solutions (molecularly disperse systems), one type of solid dispersion, are well known for use in pharmaceutical technology (see (Chiou and Riegelman, J. Pharm. Sci., 60, 1281-1300 (1971)) and are useful in increasing dissolution rates and increasing the bioavailability of poorly water-soluble drugs.

This invention also provides solid dosage forms comprising the solid solution described above. Solid dosage forms include tablets, capsules, chewable tablets and dispersible or effervescent tablets. Known excipients can be blended with the solid solution to provide the desired dosage form. For example, a capsule can contain the solid solution blended with (a) a disintegrant and a lubricant, or (b) a disintegrant, a lubricant and a surfactant. In addition, a capsule can contain a bulking agent, such as lactose or microcrystalline cellulose. A tablet can contain the solid solution blended with at least one disintegrant, a lubricant, a surfactant, a bulking agent and a glidant. A chewable tablet can contain the solid solution blended with a bulking agent, a lubricant, and if desired an additional sweetening agent (such as an artificial sweetener), and suitable flavours. Solid solutions may also be formed by spraying solutions of drug and a suitable polymer onto the surface of inert carriers such as sugar beads (‘non-pareils’). These beads can subsequently be filled into capsules or compressed into tablets.

The pharmaceutical formulations may be presented to a patient in “patient packs” containing an entire course of treatment in a single package, usually a blister pack. Patient packs have an advantage over traditional prescriptions, where a pharmacist divides a patient's supply of a pharmaceutical from a bulk supply, in that the patient always has access to the package insert contained in the patient pack, normally missing in patient prescriptions. The inclusion of a package insert has been shown to improve patient compliance with the physician's instructions.

Compositions for topical use and nasal delivery include ointments, creams, sprays, patches, gels, liquid drops and inserts (for example intraocular inserts). Such compositions can be formulated in accordance with known methods.

Examples of formulations for rectal or intra-vaginal administration include pessaries and suppositories which may be, for example, formed from a shaped moldable or waxy material containing the active compound. Solutions of the active compound may also be used for rectal administration.

Compositions for administration by inhalation may take the form of inhalable powder compositions or liquid or powder sprays and can be administrated in standard form using powder inhaler devices or aerosol dispensing devices. Such devices are well known. For administration by inhalation, the powdered formulations typically comprise the active compound together with an inert solid powdered diluent such as lactose.

The compounds of the formula (I) will generally be presented in unit dosage form and, as such, will typically contain sufficient compound to provide a desired level of biological activity. For example, a formulation may contain from 1 nanogram to 2 grams of active ingredient, e.g. from 1 nanogram to 2 milligrams of active ingredient. Within these ranges, particular sub-ranges of compound are 0.1 milligrams to 2 grams of active ingredient (more usually from 10 milligrams to 1 gram, e.g. 50 milligrams to 500 milligrams), or 1 microgram to 20 milligrams (for example 1 microgram to 10 milligrams, e.g. 0.1 milligrams to 2 milligrams of active ingredient).

For oral compositions, a unit dosage form may contain from 1 milligram to 2 grams, more typically 10 milligrams to 1 gram, for example 50 milligrams to 1 gram, e.g. 100 miligrams to 1 gram, of active compound.

The active compound will be administered to a patient in need thereof (for example a human or animal patient) in an amount sufficient to achieve the desired therapeutic effect.

### Methods of Treatment

The compounds of the formula (I) and sub-groups as defined herein may be useful in the prophylaxis or treatment of a range of disease states or conditions mediated by Polθ. Thus, according to a further aspect of the invention there is provided a method of treating a disease state or condition mediated by Polθ (e.g. cancer) which comprises administering to a subject in need thereof a compound of formula (I) as described herein. Examples of such disease states and conditions are set out above, and in particular include cancer.

The compounds are generally administered to a subject in need of such administration, for example a human or animal patient, particularly a human.

The compounds will typically be administered in amounts that are therapeutically or prophylactically useful and which generally are non-toxic. However, in certain situations (for example in the case of life threatening diseases), the benefits of administering a compound of the formula (I) may outweigh the disadvantages of any toxic effects or side effects, in which case it may be considered desirable to administer compounds in amounts that are associated with a degree of toxicity.

The compounds may be administered over a prolonged term to maintain beneficial therapeutic effects or may be administered for a short period only. Alternatively they may be administered in a continuous manner or in a manner that provides intermittent dosing (e.g. a pulsatile manner).

A typical daily dose of the compound of formula (I) can be in the range from 100 picograms to 100 milligrams per kilogram of body weight, more typically 5 nanograms to 25 milligrams per kilogram of bodyweight, and more usually 10 nanograms to 15 milligrams per kilogram (e.g. 10 nanograms to 10 milligrams, and more typically 1 microgram per kilogram to 20 milligrams per kilogram, for example 1 microgram to 10 milligrams per kilogram) per kilogram of bodyweight although higher or lower doses may be administered where required. The compound of the formula (I) can be administered on a daily basis or on a repeat basis every 2, or 3, or 4, or 5, or 6, or 7, or 10 or 14, or 21, or 28 days for example.

The compounds of the invention may be administered orally in a range of doses, for example 1 to 1500 mg, 2 to 800 mg, or 5 to 500 mg, e.g. 2 to 200 mg or 10 to 1000 mg, particular examples of doses including 10, 20, 50 and 80 mg. The compound may be administered once or more than once each day, for example one suitable dosage regime may require 1000 mg to 1500 mg two or three times per day. The compound can be administered continuously (i.e. taken every day without a break for the duration of the treatment regimen). Alternatively, the compound can be administered intermittently (i.e. taken continuously for a given period such as a week, then discontinued for a period such as a week and then taken continuously for another period such as a week and so on throughout the duration of the treatment regimen). Examples of treatment regimens involving intermittent administration include regimens wherein administration is in cycles of one week on, one week off; or two weeks on, one week off; or three weeks on, one week off; or two weeks on, two weeks off; or four weeks on two weeks off; or one week on three weeks off—for one or more cycles, e.g. 2, 3, 4, 5, 6, 7, 8, 9 or 10 or more cycles.

In one particular dosing schedule, a patient will be given an infusion of a compound of the formula (I) for periods of one hour daily for up to ten days in particular up to five days for one week, and the treatment repeated at a desired interval such as two to four weeks, in particular every three weeks.

More particularly, a patient may be given an infusion of a compound of the formula (I) for periods of one hour daily for 5 days and the treatment repeated every three weeks.

In another particular dosing schedule, a patient is given an infusion over 30 minutes to 1 hour followed by maintenance infusions of variable duration, for example 1 to 5 hours, e.g. 3 hours.

In a further particular dosing schedule, a patient is given a continuous infusion for a period of 12 hours to 5 days, an in particular a continuous infusion of 24 hours to 72 hours.

In another particular dosing schedule, a patient is given the compound orally once a week.

In another particular dosing schedule, a patient is given the compound orally once-daily for between 7 and 28 days such as 7, 14 or 28 days.

In another particular dosing schedule, a patient is given the compound orally once-daily for 1 day, 2 days, 3 days, 5 days or 1 week followed by the required amount of days off to complete a one or two week cycle.

In another particular dosing schedule, a patient is given the compound orally once-daily for 2 weeks followed by 2 weeks off.

In another particular dosing schedule, a patient is given the compound orally once-daily for 2 weeks followed by 1 week off.

In another particular dosing schedule, a patient is given the compound orally once-daily for 1 week followed by 1 week off.

Ultimately, however, the quantity of compound administered and the type of composition used will be commensurate with the nature of the disease or physiological condition being treated and will be at the discretion of the physician.

It will be appreciated that Polθ inhibitors can be used as a single agent or in combination with other anticancer agents. Combination experiments can be performed, for example, as described in Chou T C, Talalay P. Quantitative analysis of dose-effect relationships: the combined effects of multiple drugs or enzyme inhibitors. Adv Enzyme Regulat 1984;22: 27-55.

The compounds as defined herein can be administered as the sole therapeutic agent or they can be administered in combination therapy with one of more other compounds (or therapies) for treatment of a particular disease state, for example a neoplastic disease such as a cancer as hereinbefore defined. For the treatment of the above conditions, the compounds of the invention may be advantageously employed in combination with one or more other medicinal agents, more particularly, with other anti-cancer agents or adjuvants (supporting agents in the therapy) in cancer therapy. Examples of other therapeutic agents or treatments that may be administered together (whether concurrently or at different time intervals) with the compounds of the formula (I) include but are not limited to:
Topoisomerase I inhibitors;
Antimetabolites;
Tubulin targeting agents;
DNA binder and topoisomerase II inhibitors;
Alkylating Agents;
Monoclonal Antibodies;
Anti-Hormones;
Signal Transduction Inhibitors;
Proteasome Inhibitors;
DNA methyl transferase inhibitors;
Cytokines and retinoids;
Chromatin targeted therapies;
Radiotherapy; and
Other therapeutic or prophylactic agents.

Particular examples of anti-cancer agents or adjuvants (or salts thereof), include but are not limited to any of the agents selected from groups (i)-(xlvi), and optionally group (xlvii), below:
(i) Platinum compounds, for example cisplatin (optionally combined with amifostine), carboplatin or oxaliplatin;
(ii) Taxane compounds, for example paclitaxel, paclitaxel protein bound particles (Abraxane™), docetaxel, cabazitaxel or larotaxel;
(iii) Topoisomerase I inhibitors, for example camptothecin compounds, for example camptothecin, irinotecan(CPT11), SN-38, or topotecan;
(iv) Topoisomerase II inhibitors, for example anti-tumour epipodophyllotoxins or podophyllotoxin derivatives for example etoposide, or teniposide;
(v) Vinca alkaloids, for example vinblastine, vincristine, liposomal vincristine (Onco-TCS), vinorelbine, vindesine, vinflunine or vinvesir;
(vi) Nucleoside derivatives, for example 5-fluorouracil (5-FU, optionally in combination with leucovorin), gemcitabine, capecitabine, tegafur, UFT, S1, cladribine, cytarabine (Ara-C, cytosine arabinoside), fludarabine, clofarabine, or nelarabine;
(vii) Antimetabolites, for example clofarabine, aminopterin, or methotrexate, azacitidine, cytarabine, floxuridine, pentostatin, thioguanine, thiopurine, 6-mercaptopurine, or hydroxyurea (hydroxycarbamide);
(viii) Alkylating agents, such as nitrogen mustards or nitrosourea, for example cyclophosphamide, chlorambucil, carmustine (BCNU), bendamustine, thiotepa, melphalan, treosulfan, lomustine (CCNU), altretamine, busulfan, dacarbazine, estramustine, fotemustine, ifosfamide (optionally in combination with mesna), pipobroman, procarbazine, streptozocin, temozolomide, uracil, mechlorethamine, methylcyclohexylchloroethylnitrosurea, or nimustine (ACNU);
(ix) Anthracyclines, anthracenediones and related drugs, for example daunorubicin, doxorubicin (optionally in combination with dexrazoxane), liposomal formulations of doxorubicin (eg. Caelyx™, Myocet™, Doxil™), idarubicin, mitoxantrone, epirubicin, amsacrine, or valrubicin;
(x) Epothilones, for example ixabepilone, patupilone, BMS-310705, KOS-862 and ZK-EPO, epothilone A, epothilone B, desoxyepothilone B (also known as epothilone D or KOS-862), aza-epothilone B (also known as BMS-247550), aulimalide, isolaulimalide, or luetherobin;
(xi) DNA methyl transferase inhibitors, for example temozolomide, azacytidine or decitabine, or SGI-110;
(xii) Antifolates, for example methotrexate, pemetrexed disodium, or raltitrexed;
(xiii) Cytotoxic antibiotics, for example antinomycin D, bleomycin, mitomycin C, dactinomycin, carminomycin, daunomycin, levamisole, plicamycin, or mithramycin;
(xiv) Tubulin-binding agents, for example combrestatin, colchicines or nocodazole;
(xv) Signal Transduction inhibitors such as Kinase inhibitors (e.g. EGFR (epithelial growth factor receptor) inhibitors, VEGFR (vascular endothelial growth factor receptor) inhibitors, PDGFR (platelet-derived growth factor receptor) inhibitors, MTKI (multi target kinase inhibitors), Raf inhibitors, mTOR inhibitors for example imatinib mesylate, erlotinib, gefitinib, dasatinib, lapatinib, dovotinib, axitinib, nilotinib, vandetanib, vatalinib, pazopanib, sorafenib, sunitinib, temsirolimus, everolimus (RAD 001), vemurafenib (PLX4032/RG7204), dabrafenib, encorafenib or an IKB kinase inhibitor such as SAR-113945, bardoxolone, BMS-066, BMS-345541, IMD-0354, IMD-2560, or IMD-1041, or MEK inhibitors such as Selumetinib (AZD6244) and Trametinib (GSK121120212);
(xvi) Aurora kinase inhibitors for example AT9283, barasertib (AZD1152), TAK-901, MK0457 (VX680), cenisertib (R-763), danusertib (PHA-739358), alisertib (MLN-8237), or MP-470;
(xvii) CDK inhibitors for example AT7519, roscovitine, seliciclib, alvocidib (flavopiridol), dinaciclib (SCH-727965), 7-hydroxy-staurosporine (UCN-01), JNJ-7706621, BMS-387032 (a.k.a. SNS-032), PHA533533, PD332991, ZK-304709, or AZD-5438;
(xviii) PKA/B inhibitors and PKB (akt) pathway inhibitors for example AKT inhibitors such as KRX-0401 (perifosine/ NSC 639966), ipatasertib (GDC-0068; RG-7440), afuresertib (GSK-2110183; 2110183), MK-2206, MK-8156, AT13148, AZD-5363, triciribine phosphate (VQD-002; triciribine phosphate monohydrate (API-2; TCN-P; TCN-PM; VD-0002), RX-0201, NL-71-101, SR-13668, PX-316, AT13148, AZ-5363, Semaphore, SF1126, or Enzastaurin HCl (LY317615) or MTOR inhibitors such as rapamycin analogues such as RAD 001 (everolimus), CCl 779 (temsirolemus), AP23573 and ridaforolimus, sirolimus (originally known as rapamycin), AP23841 and AP23573, calmodulin inhibitors e.g. CBP-501 (forkhead translocation inhibitors), enzastaurin HCl (LY317615) or PI3K Inhibitors such as dactolisib (BEZ235), buparlisib (BKM-120; NVP-BKM-120), BYL719, copanlisib (BAY-80-6946), ZSTK-474, CUDC-907, apitolisib (GDC-0980; RG-7422), pictilisib (pictrelisib, GDC-0941, RG-7321), GDC-0032, GDC-0068, GSK-2636771, idelalisib (formerly CAL-101, GS 1101, GS-1101), MLN1117 (INK1117), MLN0128 (INK128), IPI-145 (INK1197), LY-3023414, ipatasertib, afuresertib, MK-2206, MK-8156, LY-3023414, LY294002, SF1126 or PI-103, or sonolisib (PX-866);
(xix) Hsp90 inhibitors for example AT13387, herbimycin, geldanamycin (GA), 17-allylamino-17-desmethoxygeldanamycin (17-AAG) e.g. NSC-330507, Kos-953 and CNF-1010, 17-dimethylaminoethylamino-17-demethoxygeldanamycin hydrochloride (17-DMAG) e.g. NSC-707545 and Kos-1022, NVP-AUY922 (VER-52296), NVP-BEP800, CNF-2024 (BIIB-021 an oral purine), ganetespib (STA-9090), SNX-5422 (SC-102112) or IPI-504;
(xx) Monoclonal Antibodies (unconjugated or conjugated to radioisotopes, toxins or other agents), antibody derivatives and related agents, such as anti-CD, anti-VEGFR, anti-HER2, anti-CTLA4, anti-PD-1 or anti-EGFR antibodies, for example rituximab (CD20), ofatumumab (CD20), ibritumomab tiuxetan (CD20), GA101 (CD20), tositumomab (CD20), epratuzumab (CD22), lintuzumab (CD33), gemtuzumab ozogamicin (CD33), alemtuzumab (CD52), galiximab (CD80), trastuzumab (HER2 antibody), pertuzumab (HER2), trastuzumab-DM1 (HER2), ertumaxomab (HER2 and CD3), cetuximab (EGFR), panitumumab (EGFR), necitumumab (EGFR), nimotuzumab (EGFR), bevacizumab (VEGF), catumaxumab (EpCAM and CD3), abagovomab (CA125), farletuzumab (folate receptor), elotuzumab (CS1), denosumab (RANK ligand), figitumumab (IGF1R), CP751,871 (IGF1R), mapatumumab (TRAIL receptor), metMAB (met), mitumomab (GD3 ganglioside), naptumomab estafenatox (5T4), siltuximab (IL6), or immunomodulating agents such as CTLA-4 blocking antibodies and/or antibodies against PD-1 and PD-L1 and/or PD-L2 for example ipilimumab (CTLA4), MK-3475 (pembrolizumab, formerly lambrolizumab, anti-PD-1), nivolumab (anti-PD-1), BMS-936559 (anti- PD-L1), MPDL320A, AMP-514 or MED14736 (anti-PD-L1), or tremelimumab (formerly ticilimumab, CP-675,206, anti-CTLA-4);
(xxi) Estrogen receptor antagonists or selective estrogen receptor modulators (SERMs) or inhibitors of estrogen synthesis, for example tamoxifen, fulvestrant, toremifene, droloxifene, faslodex, or raloxifene;
(xxii) Aromatase inhibitors and related drugs, such as exemestane, anastrozole, letrazole, testolactone aminoglutethimide, mitotane or vorozole;
(xxiii) Antiandrogens (i.e. androgen receptor antagonists) and related agents for example bicalutamide, nilutamide, flutamide, cyproterone, or ketoconazole;
(xxiv) Hormones and analogues thereof such as medroxyprogesterone, diethylstilbestrol (a.k.a. diethylstilboestrol) or octreotide;
(xxv) Steroids for example dromostanolone propionate, megestrol acetate, nandrolone (decanoate, phenpropionate), fluoxymestrone or gossypol,
(xxvi) Steroidal cytochrome P450 17alpha-hydroxylase-17,20-lyase inhibitor (CYP17), e.g. abiraterone;
(xxvii) Gonadotropin releasing hormone agonists or antagonists (GnRAs) for example abarelix, goserelin acetate, histrelin acetate, leuprolide acetate, triptorelin, buserelin, or deslorelin;
(xxviii) Glucocorticoids, for example prednisone, prednisolone, dexamethasone;
(xxix) Differentiating agents, such as retinoids, rexinoids, vitamin D or retinoic acid and retinoic acid metabolism blocking agents (RAMBA) for example accutane, alitretinoin, bexarotene, or tretinoin;
(xxx) Farnesyltransferase inhibitors for example tipifarnib;
(xxxi) Chromatin targeted therapies such as histone deacetylase (HDAC) inhibitors for example panobinostat, resminostat, abexinostat, vorinostat, romidepsin, belinostat, entinostat, quisinostat, pracinostat, tefinostat, mocetinostat, givinostat, CUDC-907, CUDC-101, ACY-1215, MGCD-290, EVP-0334, RG-2833, 4SC-202, romidepsin, AR-42 (Ohio State University), CG-200745, valproic acid, CKD-581, sodium butyrate, suberoylanilide hydroxamide acid (SAHA), depsipeptide (FR 901228), dacinostat (NVP-LAQ824), R306465/ JNJ-16241199, JNJ-26481585, trichostatin A, chlamydocin, A-173, JNJ-MGCD-0103, PXD-101, or apicidin;
(xxxii) Proteasome Inhibitors for example bortezomib, carfilzomib, delanzomib (CEP-18770), ixazomib (MLN-9708), oprozomib (ONX-0912) or marizomib;
(xxxiii) Photodynamic drugs for example porfimer sodium or temoporfin;
(xxxiv) Marine organism-derived anticancer agents such as trabectidin;
(xxxv) Radiolabelled drugs for radioimmunotherapy for example with a beta particle-emitting isotope (e.g. Iodine -131, Yittrium -90) or an alpha particle-emitting isotope (e.g.,

Bismuth-213 or Actinium-225) for example ibritumomab or Iodine tositumomab;
(xxxvi) Telomerase inhibitors for example telomestatin;
(xxxvii) Matrix metalloproteinase inhibitors for example batimastat, marimastat, prinostat or metastat;
(xxxviii) Recombinant interferons (such as interferon-y and interferon a) and interleukins (e.g. interleukin 2), for example aldesleukin, denileukin diftitox, interferon alfa 2a, interferon alfa 2b, or peginterferon alfa 2b;
(xxxix) Selective immunoresponse modulators for example thalidomide, or lenalidomide;
(xl) Therapeutic Vaccines such as sipuleucel-T (Provenge) or OncoVex;
(xli) Cytokine-activating agents include Picibanil, Romurtide, Sizofiran, Virulizin, or Thymosin;
(xlii) Arsenic trioxide;
(xliii) Inhibitors of G-protein coupled receptors (GPCR) for example atrasentan;
(xliv) Enzymes such as L-asparaginase, pegaspargase, rasburicase, or pegademase;
(xlv) DNA repair inhibitors such as PARP inhibitors for example, olaparib, velaparib, iniparib, rucaparib (AG-014699 or PF-01367338), talazoparib or AG-014699;
(xlvi) DNA damage response inhibitors such as ATM inhibitors AZD0156 MS3541, ATR inhibitors AZD6738, M4344, M6620 weel inhibitor AZD1775;
(xlvii) Agonists of Death receptor (e.g. TNF-related apoptosis inducing ligand (TRAIL) receptor), such as mapatumumab (formerly HGS-ETR1), conatumumab (formerly AMG 655), PRO95780, lexatumumab, dulanermin, CS-1008, apomab or recombinant TRAIL ligands such as recombinant Human TRAIL/Apo2 Ligand;
(xlviii) Prophylactic agents (adjuncts); i.e. agents that reduce or alleviate some of the side effects associated with chemotherapy agents, for example
   anti-emetic agents,
   agents that prevent or decrease the duration of chemotherapy-associated neutropenia and prevent complications that arise from reduced levels of platelets, red blood cells or white blood cells, for example interleukin-11 (e.g. oprelvekin), erythropoietin (EPO) and analogues thereof (e.g. darbepoetin alfa), colony-stimulating factor analogs such as granulocyte macrophage-colony stimulating factor (GM-CSF) (e.g. sargramostim), and granulocyte-colony stimulating factor (G-CSF) and analogues thereof (e.g. filgrastim, pegfilgrastim),
   agents that inhibit bone resorption such as denosumab or bisphosphonates e.g. zoledronate, zoledronic acid, pamidronate and ibandronate,
   agents that suppress inflammatory responses such as dexamethasone, prednisone, and prednisolone,
   agents used to reduce blood levels of growth hormone and IGF-I (and other hormones) in patients with acromegaly or other rare hormone-producing tumours, such as synthetic forms of the hormone somatostatin e.g. octreotide acetate,
   antidote to drugs that decrease levels of folic acid such as leucovorin, or folinic acid,
   agents for pain e.g. opiates such as morphine, diamorphine and fentanyl,
   non-steroidal anti-inflammatory drugs (NSAID) such as COX-2 inhibitors for example celecoxib, etoricoxib and lumiracoxib,
   agents for mucositis e.g. palifermin,
   agents for the treatment of side-effects including anorexia, cachexia, oedema or thromoembolic episodes, such as megestrol acetate.

In one embodiment the anticancer is selected from recombinant interferons (such as interferon-γ and interferon α) and interleukins (e.g. interleukin 2), for example aldesleukin, denileukin diftitox, interferon alfa 2a, interferon alfa 2b, or peginterferon alfa 2b; interferon-α2 (500 μ/ml) in particular interferon-β; and signal transduction inhibitors such as kinase inhibitors (e.g. EGFR (epithelial growth factor receptor) inhibitors, VEGFR (vascular endothelial growth factor receptor) inhibitors, PDGFR (platelet-derived growth factor receptor) inhibitors, MTKI (multi target kinase inhibitors), Raf inhibitors, mTOR inhibitors for example imatinib mesylate, erlotinib, gefitinib, dasatinib, lapatinib, dovotinib, axitinib, nilotinib, vandetanib, vatalinib, pazopanib, sorafenib, sunitinib, temsirolimus, everolimus (RAD 001), vemurafenib (PLX4032/RG7204), dabrafenib, encorafenib or an IKB kinase inhibitor such as SAR-113945, bardoxolone, BMS-066, BMS-345541, IMD-0354, IMD-2560, or IMD-1041, or MEK inhibitors such as Selumetinib (AZD6244) and Trametinib (GSK121120212), in particular Raf inhibitors (e.g. vemurafenib) or MEK inhibitors (e.g. trametinib).

Each of the compounds present in the combinations of the invention may be given in individually varying dose schedules and via different routes. As such, the posology of each of the two or more agents may differ: each may be administered at the same time or at different times. A person skilled in the art would know through his or her common general knowledge the dosing regimes and combination therapies to use. For example, the compound of the invention may be using in combination with one or more other agents which are administered according to their existing combination regimen. Examples of standard combination regimens are provided below.

The taxane compound is advantageously administered in a dosage of 50 to 400 mg per square meter (mg/m²) of body surface area, for example 75 to 250 mg/m², particularly for paclitaxel in a dosage of about 175 to 250 mg/m² and for docetaxel in about 75 to 150 mg/m² per course of treatment.

The camptothecin compound is advantageously administered in a dosage of 0.1 to 400 mg per square meter (mg/m²) of body surface area, for example 1 to 300 mg/m², particularly for irinotecan in a dosage of about 100 to 350 mg/m² and for topotecan in about 1 to 2 mg/m² per course of treatment.

The anti-tumour podophyllotoxin derivative is advantageously administered in a dosage of 30 to 300 mg per square meter (mg/m²) of body surface area, for example 50 to 250mg/m², particularly for etoposide in a dosage of about 35 to 100 mg/m² and for teniposide in about 50 to 250 mg/m² per course of treatment.

The anti-tumour vinca alkaloid is advantageously administered in a dosage of 2 to 30 mg per square meter (mg/m²) of body surface area, particularly for vinblastine in a dosage of about 3 to 12 mg/m², for vincristine in a dosage of about 1 to 2 mg/m², and for vinorelbine in dosage of about 10 to 30 mg/m² per course of treatment.

The anti-tumour nucleoside derivative is advantageously administered in a dosage of 200 to 2500 mg per square meter (mg/m²) of body surface area, for example 700 to 1500 mg/², particularly for 5-FU in a dosage of 200 to 500mg/m², for gemcitabine in a dosage of about 800 to 1200 mg/m² and for capecitabine in about 1000 to 2500 mg/m² per course of treatment.

The alkylating agents such as nitrogen mustard or nitrosourea is advantageously administered in a dosage of 100 to 500 mg per square meter (mg/m²) of body surface area, for example 120 to 200 mg/², particularly for cyclophosphamide in a dosage of about 100 to 500 mg/m², for chlorambucil in a dosage of about 0.1 to 0.2 mg/kg, for carmustine in a dosage of about 150 to 200 mg/², and for lomustine in a dosage of about 100 to 150 mg/m² per course of treatment.

The anti-tumour anthracycline derivative is advantageously administered in a dosage of 10 to 75 mg per square meter (mg/m²) of body surface area, for example 15 to 60 mg/², particularly for doxorubicin in a dosage of about 40 to 75 mg/², for daunorubicin in a dosage of about 25 to 45mg/², and for idarubicin in a dosage of about 10 to 15 mg/m² per course of treatment.

The antiestrogen agent is advantageously administered in a dosage of about 1 to 100 mg daily depending on the particular agent and the condition being treated. Tamoxifen is advantageously administered orally in a dosage of 5 to 50 mg, particularly 10 to 20 mg twice a day, continuing the therapy for sufficient time to achieve and maintain a therapeutic effect. Toremifene is advantageously administered orally in a dosage of about 60mg once a day, continuing the therapy for sufficient time to achieve and maintain a therapeutic effect. Anastrozole is advantageously administered orally in a dosage of about 1mg once a day. Droloxifene is advantageously administered orally in a dosage of about 20-100mg once a day. Raloxifene is advantageously administered orally in a dosage of about 60 mg once a day. Exemestane is advantageously administered orally in a dosage of about 25 mg once a day.

Antibodies are advantageously administered in a dosage of about 1 to 5 mg per square meter (mg/m²) of body surface area, or as known in the art, if different. Trastuzumab is advantageously administered in a dosage of 1 to 5 mg per square meter (mg/m²) of body surface area, particularly 2 to 4 mg/m² per course of treatment.

Where the compound of the formula (I) is administered in combination therapy with one, two, three, four or more other therapeutic agents (particularly one or two, more particularly one), the compounds can be administered simultaneously or sequentially. In the latter case, the two or more compounds will be administered within a period and in an amount and manner that is sufficient to ensure that an advantageous or synergistic effect is achieved. When administered sequentially, they can be administered at closely spaced intervals (for example over a period of 5-10 minutes) or at longer intervals (for example 1, 2, 3, 4 or more hours apart, or even longer periods apart where required), the precise dosage regimen being commensurate with the properties of the therapeutic agent(s). These dosages may be administered for example once, twice or more per course of treatment, which may be repeated for example every 7, 14, 21 or 28 days.

In one embodiment is provided a compound of formula (I) for the manufacture of a medicament for use in therapy wherein said compound is used in combination with one, two, three, or four other therapeutic agents. In another embodiment is provided a medicament for treating cancer which comprises a compound of formula (I) wherein said medicament is used in combination with one, two, three, or four other therapeutic agents. The invention further provides use of a compound of formula (I) for the manufacture of a medicament for enhancing or potentiating the response rate in a patient suffering from a cancer where the patient is being treated with one, two, three, or four other therapeutic agents.

It will be appreciated that the particular method and order of administration and the respective dosage amounts and regimes for each component of the combination will depend on the particular other medicinal agent and compound of the present invention being administered, their route of administration, the particular tumour being treated and the particular host being treated. The optimum method and order of administration and the dosage amounts and regime can be readily determined by those skilled in the art using conventional methods and in view of the information set out herein.

The weight ratio of the compound according to the present invention and the one or more other anticancer agent(s) when given as a combination may be determined by the person skilled in the art. Said ratio and the exact dosage and frequency of administration depends on the particular compound according to the invention and the other anticancer agent(s) used, the particular condition being treated, the severity of the condition being treated, the age, weight, gender, diet, time of administration and general physical condition of the particular patient, the mode of administration as well as other medication the individual may be taking, as is well known to those skilled in the art. Furthermore, it is evident that the effective daily amount may be lowered or increased depending on the response of the treated subject and/or depending on the evaluation of the physician prescribing the compounds of the instant invention. A particular weight ratio for the present compound of formula (I) and another anticancer agent may range from 1/10 to 10/1, more in particular from 1/5 to 5/1, even more in particular from 1/3 to 3/1.

The compounds of the invention may also be administered in conjunction with non-chemotherapeutic treatments such as radiotherapy, photodynamic therapy, gene therapy; surgery and controlled diets.

The compounds of the present invention also have therapeutic applications in sensitising tumour cells for radiotherapy and chemotherapy. Hence the compounds of the present invention can be used as “radiosensitizer” and/or “chemosensitizer” or can be given in combination with another “radiosensitizer” and/or “chemosensitizer”. In one embodiment the compound of the invention is for use as chemosensitiser.

The term “radiosensitizer” is defined as a molecule administered to patients in therapeutically effective amounts to increase the sensitivity of the cells to ionizing radiation and/or to promote the treatment of diseases which are treatable with ionizing radiation.

The term “chemosensitizer” is defined as a molecule administered to patients in therapeutically effective amounts to increase the sensitivity of cells to chemotherapy and/or promote the treatment of diseases which are treatable with chemotherapeutics.

In one embodiment the compound of the invention is administered with a “radiosensitizer” and/or “chemosensitizer”. In one embodiment the compound of the invention is administered with an “immune sensitizer”.

The term “immune sensitizer” is defined as a molecule administered to patients in therapeutically effective amounts to increase the sensitivity of cells to a Polθ inhibitor.

Many cancer treatment protocols currently employ radiosensitizers in conjunction with radiation of x-rays. Examples of x-ray activated radiosensitizers include, but are not limited to, the following: metronidazole, misonidazole, desmethylmisonidazole, pimonidazole, etanidazole, nimorazole, mitomycin C, RSU 1069, SR 4233, EO9, RB 6145, nicotinamide, 5-bromodeoxyuridine (BUdR), 5- iododeoxyuridine (lUdR), bromodeoxycytidine, fluorodeoxyuridine (FudR), hydroxyurea, cisplatin, and therapeutically effective analogs and derivatives of the same.

Photodynamic therapy (PDT) of cancers employs visible light as the radiation activator of the sensitizing agent. Examples of photodynamic radiosensitizers include the following, but are not limited to: hematoporphyrin derivatives, Photofrin, benzoporphyrin derivatives, tin etioporphyrin, pheoborbide-a, bacteriochlorophyll-a, naphthalocyanines, phthalocyanines, zinc phthalocyanine, and therapeutically effective analogs and derivatives of the same.

Radiosensitizers may be administered in conjunction with a therapeutically effective amount of one or more other compounds, including but not limited to: compounds of the invention; compounds which promote the incorporation of radiosensitizers to the target cells;

compounds which control the flow of therapeutics, nutrients, and/or oxygen to the target cells; chemotherapeutic agents which act on the tumour with or without additional radiation; or other therapeutically effective compounds for treating cancer or other diseases.

Chemosensitizers may be administered in conjunction with a therapeutically effective amount of one or more other compounds, including but not limited to: compounds of the invention; compounds which promote the incorporation of chemosensitizers to the target cells; compounds which control the flow of therapeutics, nutrients, and/or oxygen to the target cells; chemotherapeutic agents which act on the tumour or other therapeutically effective compounds for treating cancer or other disease. Calcium antagonists, for example verapamil, are found useful in combination with antineoplastic agents to establish chemosensitivity in tumor cells resistant to accepted chemotherapeutic agents and to potentiate the efficacy of such compounds in drug-sensitive malignancies.

Examples of immune sensitizers include the following, but are not limited to: immunomodulating agents, for example monoclonal antibodies such as immune checkpoint antibodies [e.g. CTLA-4 blocking antibodies and/or antibodies against PD-1 and PD-L1 and/or PD-L2 for example ipilimumab (CTLA4), MK-3475 (pembrolizumab, formerly lambrolizumab, anti-PD-1), nivolumab (anti-PD-1), BMS-936559 (anti- PD-L1), MPDL320A, AMP-514 or MED14736 (anti-PD-L1), or tremelimumab (formerly ticilimumab, CP-675,206, anti-CTLA-4)]; or Signal Transduction inhibitors; or cytokines (such as recombinant interferons); or oncolytic viruses; or immune adjuvants (e.g. BCG).

Immune sensitizers may be administered in conjunction with a therapeutically effective amount of one or more other compounds, including but not limited to: compounds of the invention; compounds which promote the incorporation of immune sensitizers to the target cells; compounds which control the flow of therapeutics, nutrients, and/or oxygen to the target cells; therapeutic agents which act on the tumour or other therapeutically effective compounds for treating cancer or other disease.

For use in combination therapy with another chemotherapeutic agent, the compound of the formula (I) and one, two, three, four or more other therapeutic agents can be, for example, formulated together in a dosage form containing two, three, four or more therapeutic agents i.e. in a unitary pharmaceutical composition containing all agents. In an alternative embodiment, the individual therapeutic agents may be formulated separately and presented together in the form of a kit, optionally with instructions for their use.

In one embodiment is provided a combination of a compound of formula (I) with one or more (e.g. 1 or 2) other therapeutic agents (e.g. anticancer agents as described above). In a further embodiment is provided a combination of a Polθ inhibitor as described herein and a PI3K/AKT pathway inhibitor selected from: apitolisib, buparlisib, Copanlisib, pictilisib, ZSTK-474, CUDC-907, GSK-2636771, LY-3023414, ipatasertib, afuresertib, MK-2206, MK-8156, Idelalisib, BEZ235 (dactolisib), BYL719, GDC- 0980, GDC-0941, GDC-0032 and GDC-0068.

In another embodiment is provided a compound of formula (I) in combination with one or more (e.g. 1 or 2) other therapeutic agents (e.g. anticancer agents) for use in therapy, such as in the prophylaxis or treatment of cancer.

In one embodiment the pharmaceutical composition comprises a compound of formula (I) together with a pharmaceutically acceptable carrier and optionally one or more therapeutic agent(s).

In another embodiment the invention relates to the use of a combination according to the invention in the manufacture of a pharmaceutical composition for inhibiting the growth of tumour cells.

In a further embodiment the invention relates to a product containing a compound of formula (I) and one or more anticancer agent, as a combined preparation for simultaneous, separate or sequential use in the treatment of patients suffering from cancer.

### EXAMPLES

The invention will now be illustrated, but not limited, by reference to the specific embodiments described in the following examples.

### ABBREVIATIONS

aq. Aqueous
Bn Benzyl
BOC tert-butyloxycarbonyl
DCM Dichloromethane
DMF Dimethylformamide
GC Gas chromatography
HDPE High-density polyethylene
HPLC High-performance liquid chromatography
LDPE Low-density polyethylene
LOD Loss on drying
MeOH Methanol
MPa Megapascal
Ms Mesylate/Methanesulfonate
MTBE Methyl tert-butyl ether
Q-NMR Quantitative Nuclear magnetic resonance
TBS tert-Butyldimethylsilyl
TEA Triethylamine
THF Tetrahydrofuran
V Volumes
w/w Weight for weight

### Intermediate 1: (3aS,4S,6aS)-2,2-Dimethyl-6-oxotetrahydro-4H-[1,3]dioxolo[4,5-c]pyrrole-4-carboxylic acid

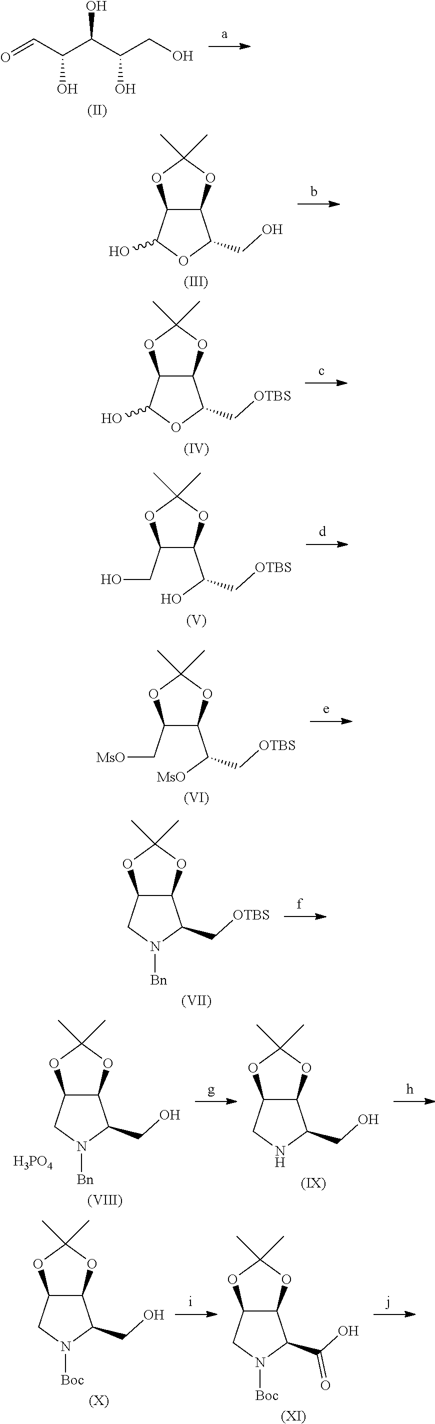

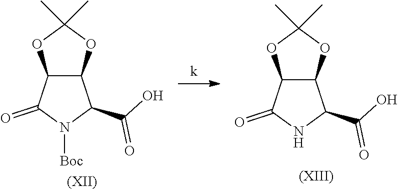

### Step a

1. Under nitrogen atmosphere, acetone (2.0 V, 100 kg) was charged to a reactor and stirring started. Additional acetone (3.0 V, 157 kg) and L-ribose (1.0 eq, 65 kg) was charged to the reactor and cooled to −3±3° C.
2. Sulfuric acid (0.07 eq, 3.25 kg) was charged dropwise to the reactor at −3±3° C. The reaction was stirred for at least 24 hours at −3±3° C., until the assay of (3aS,6S,6aS)-6-(hydroxymethyl)-2,2-dimethyltetrahydrofuro[3,4-d]dioxol-4-ol was ≥20%, measured by GC analysis.
3. TEA (0.14 eq, 5.85 kg) was charged to quench the reaction at −3±3° C. and stirred for at least 30 minutes, to adjust the pH to ≥7.
4. The reaction mixture was concentrated until the volume was 2-3V (actual volume was 148 L), while controlling the reactor inner temperature to no more than 30° C. (jacket temperature no more than 40° C.).
5. DCM (5.0 V, 443 kg) was charged to the reactor. The solution was concentrated until the volume was 2˜3V (actual volume was 148 L), controlling the temperature as described above.
6. DCM (5.0 V, 432 kg) was charged to the reactor. The solution was concentrated until the volume was 2˜3V (actual volume was 150 L), controlling the temperature as described above.
7. DCM (5.0 V, 434 kg) was charged to the reactor. The solution was concentrated until the volume was 2˜3V (actual volume was 165 L), controlling the temperature as described above.
8. (3aS,6S,6aS)-6-(Hydroxymethyl)-2,2-dimethyltetrahydrofuro[3,4-d] [1,3]dioxol-4-ol was collected as a DCM solution (207.6 kg, 82.7% purity, 36.1% Q-NMR and 90.5% yield) and stored in a drum.

The above process was repeated using L-ribose (65 kg) to provide a second batch of (3aS,6S,6aS)-6-(hydroxymethyl)-2,2-dimethyltetrahydrofuro[3,4-d] [1,3]dioxol-4-ol DCM solution (214 kg 86.3% purity, 35.1% Q-NMR and 91.2% yield).

### Step b

1. Under nitrogen atmosphere, DCM (5.0 V, 484.10 kg) was charged to a reactor and stirring started.
2. (3aS,6S,6aS)-6-(hydroxymethyl)-2,2-dimethyltetrahydrofuro[3,4-d] [1,3]dioxol-4-ol DCM solution (1.0 eq, 205.35 kg) was transferred from the drum to the reactor. The drum was rinsed with DCM (0.5 V, 48.75 kg) and the washing liquid was transferred to the reactor.
3. The DCM solution was cooled to 5±5° C.
4. TEA (2.0 eq, 78.65 kg) was charged to the reactor at 5±5° C., over at least 1 hour, followed by TBSCl (1.1 eq, 64.96 kg). The reaction was stirred for at least 2 hours at 5±5° C., until the assay of (3aS,6S,6aS)-6-(hydroxymethyl)-2,2-dimethyltetrahydrofuro[3,4-d] [1,3]dioxol-4-ol was ≤2%, measured by GC analysis.
5. The reaction mixture was cooled to −5±5° C. A 7.4% solution of citric acid (5.0 V, 388.6 kg) was charged to reactor at 0±10° C. and stirred for at least 30 minutes. The mixture was allowed to stand for at least 30 minutes, separated and the organic phase was collected.
6. A 10% solution of sodium chloride (5.0 V, 417.49 kg) was charged to reactor with organic phase at 10±10° C. and stirred for at least 30 minutes. The mixture was allowed to stand for at least 30 minutes, separated and the organic phase was collected.
7. The solution was concentrated until the volume was 2-3V (actual volume was 168 L), while controlling the reactor inner temperature to no more than 40° C. (jacket temperature no more than 55° C.).
8. THF (5.0 V, 328.15 kg) was charged to the reactor. The solution was concentrated until the volume was 2˜3V (actual volume was 165 L), while controlling the reactor inner temperature to no more than 30° C. (jacket temperature no more than 40° C.).
9. THF (5.0V, 340.25 kg) was charged to the reactor. The solution was concentrated until the volume was 2˜3V (actual volume was 210 L), controlling the temperature as described above.
10. (3aS,6S,6aS)-6-(((tert-Butyldimethylsilyl)oxy)methyl)-2,2-dimethyltetrahydrofuro[3,4-d] [1,3]dioxo1-4-ol was collected as a THF solution (226.65 kg, 91.6% purity, 42.4% assay and 80.9% yield) and stored in a drum.

The above process was repeated using (3aS,6S,6aS)-6-(hydroxymethyl)-2,2-dimethyltetrahydrofuro[3,4-d] [1,3]dioxol-4-ol in DCM solution (214 kg) to provide a second batch of (3aS,6S,6aS)-6-(((tert-butyldimethylsilyl)oxy)methyl)-2,2-dimethyltetrahydrofuro[3,4-d][1,3]dioxo1-4-ol THF solution (259.75 kg, 91.8% purity, 40.8% assay and 88.1% yield).

### Step c

1. THF (4.0 V, 376.90 kg) was charged to a reactor under nitrogen atmosphere and stirring started. Water (0.5 V, 53.85 kg) was charged to the reactor.
2. (3aS,6S,6aS)-6-(((tert-Butyldimethylsilyl)oxy)methyl)-2,2-dimethyltetrahydrofuro[3,4-d][1,3]dioxol-4-ol THF solution (1.0 eq, 259.75 kg) was transferred from the drum to the reactor.
3. The THF solution was cooled to 0-5° C.
4. Sodium borohydride (0.7 eq, 9.50 kg) was charged to the reactor in batches (no less than 10 batches) at 5±5° C. The reaction was stirred for at least 3 h at 5±5° C.
5. A 10% aq. solution of ammonium chloride (5.0 V, 530.45 kg) was charged to the reactor at 5±5° C. to quench the reaction, the addition time was not less than 3 h. The mixture was stirred and purged with nitrogen from the bottom of the reactor for at least 2 h until the residual hydrogen was completely exhausted.
6. The mixture was concentrated until the volume was 6-8V (actual volume was 780 L), while controlling the reactor inner temperature to no more than 30° C. (jacket temperature no more than 40° C.).
7. MTBE (5.0V, 386.35 kg) was charged to the reactor at 20±10° C. and stirred for at least 15 minutes at 25±5° C. The mixture was allowed to stand for at least 30 minutes, separated and the organic phase was collected.
8. The above process was repeated using (3aS,6S,6aS)-6-(((tert-butyldimethylsilyl)oxy)methyl)-2,2-dimethyltetrahydrofuro[3,4-d] [1,3]dioxol-4-ol solution (226.65 kg) and the two batches were combined for the work-up.
9. The combined MTBE solutions were concentrated until the volume was 2˜3V (actual volume was 450 L), while controlling the reactor inner temperature to no more than 30° C. (jacket temperature no more than 40° C.).
10. n-Heptane (5.0V, 682.75 kg) was charged dropwise to the reactor. The mixture was concentrated until the volume was 2˜3V (actual volume was 530 L), controlling the temperature as described above.
11. The mixture was heated to 45±5° C. (it is recommended to raise temperature by 10±5° C. per hour) and stirred for at least 1 h until all the solid had dissolved. The solution was cooled to 0±5° C. slowly (it is recommended to reduce the temperature by 10±5° C. per hour) and stirred for at least 1 hour at 0±5° C.
12. The suspension was centrifuged, and the cake was washed with n-heptane (1.0V, 137.45 kg). The filter cake was collected. The material was not dried further as LOD=0.49% after centrifugation.
13. (S)-2-((tert-Butyldimethylsilyl)oxy)-1-((4S,5R)-5-(hydroxymethyl)-2,2-dimethyl-1,3-dioxolan-4-Aethan-1-ol was obtained as a solid (176.9 kg of solid, 100% purity, 99.8% Q-NMR and 52.65 kg of DCM solution with 20% potency and the total yield is 92.0% yield).

### Steps d, e

1. DCM (9.5 V, 1290.5 kg) was charged to a reactor under nitrogen atmosphere and stirring started.
2. (S)-2-((tert-Butyldimethylsilyl)oxy)-1-((4S,5R)-5-(hydroxymethyl)-2,2-dimethyl-1,3-dioxolan-4-Aethan-1-ol (1.0 eq, 83.18 kg of solid and 52.65 kg of DCM solution with 20% potency, 135.83 kg total weight) was charged to the reactor and the temperature was reduced to 10±10° C. TEA (4.2 eq, 130.55 kg) was charged to the reactor at 10±10 ° C.
3. The reaction mixture was cooled to 0±10° C. Methanesulfonic anhydride (3.0 eq, 158.84 kg) was charged to the reactor in batches (no less than 10 batches) at 0±10° C., allowing at least 15 minutes between batches.
4. The reaction was stirred for at least 2 hours at 0±10° C., until the assay of either ((4R,5S)-5-((S)-2-((tert-butyldimethylsilyl)oxy)-1-hydroxyethyl)-2,2-dimethyl-1,3-dioxolan-4-yl)methyl methanesulfonate or (S)-2-((tert-butyldimethylsilyl)oxy)-1-(4R,5R)-5-(hydroxymethyl)-2,2-dimethyl-1,3-dioxolan-4-Aethyl methanesulfonate %, measured by HPLC analysis.
5. Water (10.0 V, 935.00 kg) was charged to reactor at 0±10° C.
6. The temperature was adjusted to 15±5° C. and the mixture stirred for at least 15 min. The mixture was allowed to stand for at least 30 minutes, separated and the organic phase was collected.
7. A 10% solution of sodium chloride (5.0 V, 473.81 kg) was charged to reactor with the organic phase at 10±10° C. and stirred for at least 15 minutes. The mixture was allowed to stand for at least 30 minutes, separated and the organic phase was collected.
8. The mixture was concentrated until the volume was 218 3V (actual volume was 230 L), while controlling the reactor inner temperature to no more than 30° C. (jacket temperature no more than 40° C.).
9. Toluene (2.0V, 168.00 kg) was charged to the reactor. The solution was concentrated until the volume was 2-3V (actual volume was 215 L), while controlling the reactor inner temperature to no more than 60° C. (jacket temperature no more than 70° C.).
10. Under nitrogen atmosphere, benzylamine (12.0 eq, 395.20 kg) was charged to the reactor with (S)-2-((tert-butyldimethylsilyl)oxy)-1-((4R,5R)-2,2-dimethyl-5-(((methylsulfonyl)oxy)methyl)-1,3-dioxolan-4-yl)ethyl methanesulfonate in toluene solution and stirring was started.
11. The reaction was heated to 90±5° C. and stirred for at least 48 hours at 90±5° C., until the assay of (S)-2-((tert-butyldimethylsilyl)oxy)-1-((4R,5R)-2,2-dimethyl-5-(((methylsulfonyl)oxy)methyl)-1,3-dioxolan-4-yl)ethyl methanesulfonate ≤1%, measured by HPLC analysis.
12. The reaction mixture was cooled to 10±10 ° C. n-Heptane (10.0 V, 643.60 kg) was charged to the reactor followed by a 10% aq. solution of citric acid (10.0 V, 832.40 kg) and the mixture was stirred for at least 30 minutes. The mixture was allowed to stand for at least 30 minutes, separated and the organic phase was collected.
13. A 10% aq. solution of citric acid (5.8 V, typically 5.5-6.5 V) was charged to the reactor to adjust the pH to 4-6. The mixture was stirred for at least 15 minutes and the pH test was repeated. The mixture was stirred for at least 30 minutes and then allowed to stand for at least 30 minutes, separated and the organic phase was collected.
14. A 10% solution of sodium chloride (5.0 V, 471.85 kg) was charged to reactor with organic phase and stirred for at least 30 minutes. The mixture was allowed to stand for at least 30 minutes, separated and the organic phase was collected.
15. The mixture was concentrated until the volume was 2˜3V (actual volume was 200 L), while controlling the reactor inner temperature to no more than 60° C. (jacket temperature no more than 70° C.).
16. THF (5.0 V, 415.10 kg) was charged to the reactor. The solution was concentrated until the volume was 2˜3V (actual volume was 190 L), while controlling the reactor inner temperature to no more than 60° C. (jacket temperature no more than 70° C.).
17. THF (5.0V, 418.05 kg) was charged to the reactor. The solution was concentrated until the volume was 2˜3V (actual volume was 280 L), controlling the temperature as described above.
18. (3aS,4R,6aR)-5-Benzyl-4-(((tert-butyldimethylsilyl)oxy)methyl)-2,2-dimethyltetrahydro-4H-[1,3]dioxolo[4,5-c]pyrrole was collected as a THF solution (267.45 kg, 96.2% purity, 33.2% Q-NMR and 77.0% yield over two steps) and stored in a drum.

The above process was repeated using (S)-2-((tert-butyldimethylsilyl)oxy)-1-((4S,5R)-5-(hydroxymethyl)-2,2-dimethyl-1,3-dioxolan-4-Aethan-1-ol (93.5 kg) to provide a second batch of (3aS,4R,6aR)-5-benzyl-4-(((tert-butyldimethylsilyl)oxy)methyl)-2,2-dimethyltetrahydro-4H-[1,3]dioxolo[4,5-c]pyrrole THF solution (198.40 kg, 99.5% purity, 50.2% Q-NMR and 86.5% yield over two steps).

### Step f

1. THF (3.0 V, 238.55 kg) was charged to a reactor under nitrogen atmosphere, stirring started and cooled to 0-5° C.
2. (3aS,4R,6aR)-5-Benzyl-4-(((tert-butyldimethylsilyl)oxy)methyl)-2,2-dimethyltetrahydro-4H-[1,3]dioxolo[4,5-c]pyrrole THF solution (1.0 eq, 267.45 kg) was transferred to the reactor at 5±5° C.
3. 85% Phosphoric acid (1.5 eq, 40.93 kg) was charged dropwise to the reactor and stirred for at least 1 hour at 5±5° C.
4. The reaction mixture was heated to 25±5° C. and stirred for at least 24 hours at 25±5° C., until the assay of (3aS,4R,6aR)-5-benzyl-4-(((tert-butyldimethylsilyl)oxy)methyl)-2,2-dimethyltetrahydro-4H-[1,3]dioxolo[4,5-c]pyrrole ≤1%, measured by HPLC analysis.
5. The mixture was cooled to 5±5° C. and stirred for at least 1 h at 5±5° C. The suspension was centrifuged, and the cake was washed with THF (1.0V, 80 kg). The filter cake was collected and transferred to a vacuum oven.
6. The cake was dried under vacuum at 35±5° C., P<_-0.08 MPa for at least 6 hours, turning at least every 2 hours and sampled for LOD until LOD≤5% (LOD=0.5%).
7. ((3aS,4R,6aR)-5-Benzyl-2,2-dimethyltetrahydro-4H-[1,3]dioxolo[4,5-c]pyrrol-4-Amethanol phosphate salt was obtained as a solid (79.7 kg, 100% purity, 99.8% Q-NMR and 72% yield over three steps).

The above process was repeated using (3aS,4R,6aR)-5-benzyl-4-(((tert-butyldimethylsilyl)oxy)methyl)-2,2-dimethyltetrahydro-4H-[1,3]dioxolo[4,5-c]pyrrole THF solution (198.40 kg) to provide a second batch of ((3aS,4R,6aR)-5-benzyl-2,2-dimethyltetrahydro-4H-[1,3]dioxolo[4,5-c]pyrrol-4-yl)methanol phosphate salt (88.44kg, 100% purity, 99.9% Q-NMR and 80% yield over three steps).

### Steps g, h

1. Methanol (7.00 V, 387.25 kg), ((3aS,4R,6aR)-5-benzyl-2,2-dimethyltetrahydro-4H-[1,3]dioxolo[4,5-c]pyrrol-4-yl)methanol phosphate salt (1.00 eq. 70.15 kg) and TEA (1.50 eq. 29.40 kg) were charged to a reactor under nitrogen atmosphere.
2. The temperature was adjusted to 25±5° C., the mixture stirred until dissolved.
3. The solution was transferred through an activated carbon filter, the filter was washed with MeOH (2 V, 110.65 kg) and combined.
4. The autoclave was purged 5 times with nitrogen. Each time the pressure was increased to 0.5 MPa and released to 0.1 MPa. The filtered solution was charged to the autoclave.
5. Palladium on carbon (6% w/w, 4.949 kg) was charged to the autoclave, the addition funnel and charging port was rinsed with methanol (1.00 V, 48.75 kg).
6. The air in the autoclave was replaced with nitrogen and hydrogen successively, hydrogen was charged to 0.5-0.8 MPa, and the reaction mixture was heated to 60±5° C.
7. The temperature was controlled at 60±5° C. and the pressure at no more than 0.8 MPa, repeat the procedure until the system pressure change is less than 0.1 MPa in one hour.

8. The reaction was stirred for at least 24 hours, until the assay of ((3aS,4R,6aR)-5-benzyl-2,2-dimethyltetrahydro-4H-[1,3]dioxolo[4,5-c]pyrrol-4-Amethanol ≤1%, measured by HPLC analysis.
9. The autoclave was purged with nitrogen. TEA (2.00 eq. 39.20 kg) and di-tert-butyl dicarbonate (1.20 eq. 50.50 kg) were charged to the reactor at 0-30° C. The reaction mixture was stirred for at least 3 h at 25±5° C. until the assay of ((3aS,4R,6aR)-2,2-dimethyltetrahydro-4H-[1,3]dioxolo[4,5-c]pyrrol-4-yl)methanol ≤1%, measured by HPLC analysis.
10. The reaction mixture was filtered under nitrogen atmosphere. The filter was washed with MeOH (3.00 V, 159.6 kg) and the filtrate was collected.
11. The solution was concentrated until the volume was 4-5V (actual volume was 330 L), while controlling the reactor inner temperature to no more than 50° C. (jacket temperature no more than 55° C.).
12. MTBE (10.00 V, 519.65 kg) and water (10.00 V, 701.00 kg) were charged successively to the reactor, the temperature adjusted to 25±5° C. and stirred for at least 15 minutes. The mixture was allowed to stand for at least 30 minutes, separated and the organic phase was collected.
13. MTBE (10.00 V, 520.45 kg) was charged to the reactor containing the aqueous phase, and stirred for at least 15 minutes at 25±5° C. The mixture was allowed to stand for at least 30 minutes, separated and the organic phase was collected.
14. The organic phases were combined and concentrated until the volume was 3˜4V (actual volume was 235 L), while controlling the reactor inner temperature to no more than 50° C. (jacket temperature no more than 55° C.).
15. Acetonitrile (10.00 V, 548.90 kg) was charged to the reactor and concentrated until the volume was 3˜4V (actual volume was 220 L), while controlling the reactor inner temperature to no more than 50° C. (jacket temperature no more than 55° C.).
16. Acetonitrile (10.00 V, 551.55 kg) was charged to the reactor. The solution was concentrated until the volume was 3˜4V (actual volume was 230 L), controlling the temperature as described above.
17. tert-Butyl (3aS,4R,6aR)-4-(hydroxymethyl)-2,2-dimethyltetrahydro-5H-[1,3]dioxolo[4,5-c]pyrrole-5-carboxylate was collected as an acetonitrile solution (215 kg, 97.1% purity, 24.1% assay and 98.0% yield) and stored in a drum at room temperature. tert-Butyl (3aS,4R,6aR)-4-(hydroxymethyl)-2,2-dimethyltetrahydro-5H-[1,3]dioxolo[4,5-c]pyrrole-5-carboxylate was used directly after qualified.

The above process was repeated using ((3aS,4R,6aR)-5-benzyl-2,2-dimethyltetrahydro-4H-[1,3]dioxolo[4,5-c]pyrrol-4-yl)methanol phosphate salt (62 kg) to provide a second batch of tert-butyl (3aS,4R,6aR)-4-(hydroxymethyl)-2,2-dimethyltetrahydro-5H-[1,3]dioxolo[4,5-c]pyrrole-5-carboxylate acetonitrile solution (198 kg, 93.0% purity, 24.9% assay and 105% yield).

### Step i

1. Water (12.0 V, 608 kg) and acetonitrile (4.0 V, 158.75 kg) were charged to a reactor under nitrogen atmosphere and stirring started.
2. tert-Butyl (3aS,4R,6aR)-4-(hydroxymethyl)-2,2-dimethyltetrahydro-5H-[1,3]dioxolo[4,5-c]pyrrole-5-carboxylate acetonitrile solution (1.0eq, 50.61 kg) was charged to the reactor and cooled to -5-0° C.
3. Ruthenium trichloride hydrate (0.03 eq, 1.27 kg) was charged to reactor at 0±5° C.
4. Sodium periodate (2.2eq, 87.15 kg) was charged to the reactor in batches (no less than 10 batches) at 0±5° C., allowing at least 10 minutes between batches.
5. The reaction was stirred for at least 2 hours at 0±5° C., until the assay of tert-butyl (3aS,4R,6aR)-4-(hydroxymethyl)-2,2-dimethyltetrahydro-5H-[1,3]dioxolo[4,5-c]pyrrole-5-carboxylate %, measured by H PLC analysis.
6. Methanol (1.0 V, 38.05 kg) and diatomite (50% w/w, 25.20 kg) were charged to the reactor at 0±5° C. and stirred for at least 15 mins at 0±5° C.
7. The mixture was filtered and the cake was washed with ethyl acetate (5.0 V, 177.75 kg).
8. Ethyl acetate (10.0 V, 444.80 kg) was charged into the filter solution and stirred for at least 15 mins at 20±5° C. The mixture was allowed to stand for at least 30 minutes, separated and the organic phase was collected.
9. Ethyl acetate (10.0 V, 455.65 kg) was charged to the reactor containing the aqueous phase, and stirred for at least 15 minutes at 25±5° C. The mixture was allowed to stand for at least 30 minutes, separated and the organic phase was collected.
10. A 15% aq. solution of sodium hydrogen sulfite (3.0 V, 186.20 kg) was charged to the reactor containing the combined organic phases and stirred for at least 15 minutes. The mixture was allowed to stand for at least 30 minutes at 20±5° C., separated and the organic phase was collected.
11. A 15% aq. solution of sodium chloride (5.0 V, 257.80 kg) was charged to the reactor containing the organic phase and stirred for at least 15 minutes. The mixture was allowed to stand for at least 30 minutes at 20±5° C., separated and the organic phase was collected.
12. The organic phase was concentrated until the volume was 7˜8V (actual volume was 360 L), while controlling the reactor inner temperature to no more than 40° C. (jacket temperature no more than 50° C.).
13. n-Heptane (10.0 V, 343.90 kg) was charged to the reactor and concentrated until the volume was 7˜8V (actual volume was 399 L), while controlling the reactor inner temperature to no more than 40° C. (jacket temperature no more than 50° C.).
14. n-Heptane (10.0 V, 335.90 kg) was charged to the reactor. The mixture was concentrated until the volume was 7-8V (actual volume was 399 L), controlling the temperature as described above.
15. n-Heptane (10.0 V, 344.10 kg) was charged to the reactor and stirred for at least 30 mins at 20±5° C.
16. The suspension was centrifuged, and the cake was washed with n-heptane (5.0 V, 172.07 kg). The filter cake was collected and transferred to a vacuum oven.
17. The cake was dried under vacuum at 40±5° C., P≤−0.08 MPa for at least 6 hours, turning at least every 2 hours and sampled for LOD until LOID5c)/o.
18. (3aS,4S,6aR)-5-(tert-Butoxycarbonyl)-2,2-dimethyltetrahydro-4H-[1,3]dioxolo[4,5-c]pyrrole-4-carboxylic acid was obtained as a solid (39.8 kg, 94.4% purity, 100% Q-NMR and 71.5% yield over two steps).

The above process was repeated using tert-butyl (3aS,4R,6aR)-4-(hydroxymethyl)-2,2-dimethyltetrahydro-5-H-[1,3]dioxolo[4,5-c]pyrrole-5-carboxylate acetonitrile solution (50 kg) to provide a second batch of (3aS,4S,6aR)-5-(tert-butoxycarbonyI)-2,2-dimethyltetrahydro-4H-[1,3]dioxolo[4,5-c]pyrrole-4-carboxylic acid (37.5 kg, 92.4% purity, 100% Q-NMR and 75.8% yield over two steps).

### Step j

1. Water (10.0 V, 403.00 kg) and acetonitrile (10.0 V, 314.45 kg) were charged to a reactor under nitrogen atmosphere and stirring started.
2. Ruthenium dioxide (0.1eq, 22.120 kg) and sodium periodate (4.5eq, 133.90 kg) were charged to the reactor at 20±5° C. The mixture was stirred for at least 30 mins at 20±5° C.
3. (3aS,4S,6aR)-5-(tert-Butoxycarbonyl)-2,2-dimethyltetrahydro-4H-[1,3]dioxolo[4,5-c]pyrrole-4-carboxylic acid (1.0eq, 39.8 kg) was charged to the reactor at 20±5° C.
4. The reaction was stirred for at least 24 hours at 20±5° C., until the assay of (3aS,4S,6aR)-5-(tert-butoxycarbonyl)-2,2-dimethyltetrahydro-4H-[1,3]dioxolo[4,5-c]pyrrole-4-carboxylic acid ≤3%, measured by HPLC analysis.
5. Methanol (1.0 V, 31.60 kg) was charged to the reactor at 15±5° C. and stirred for at least 15 mins at 15±5° C.
6. The mixture was filtered and the cake was washed with ethyl acetate (2.0 V, 59.55 kg).
7. Ethyl acetate (5.0 V, 179.80 kg) was charged into the filter solution and stirred for at least 15 mins at 15±5° C. The mixture was allowed to stand for at least 30 minutes, separated and the organic phase was collected.
8. Ethyl acetate (5.0 V, 177.65 kg) was charged to the reactor containing the aqueous phase, and stirred for at least 15 minutes at 15±5° C. The mixture was allowed to stand for at least 30 minutes, separated and the organic phase was collected.
9. Ethyl acetate (5.0 V, 178.85 kg) was charged to the reactor containing the aqueous phase, and stirred for at least 15 minutes at 15±5° C. The mixture was allowed to stand for at least 30 minutes, separated and the organic phase was collected.
10. A 10% aq. solution of sodium hydrogen sulfite (4.45 V) was charged to the reactor containing the combined organic phases and stirred for at least 15 minutes. The mixture was allowed to stand for at least 30 minutes at 15±5° C., separated and the organic phase was collected.
11. Ethyl acetate (5.0 V, 178.65 kg) was charged to the reactor containing the aqueous phase, and stirred for at least 15 minutes at 15±5° C. The mixture was allowed to stand for at least 30 minutes, separated and the organic phase was collected.
12. A 5% aq. solution of sodium chloride (5.0 V) was charged to the reactor containing the combined organic phases and stirred for at least 15 minutes. The mixture was allowed to stand for at least 30 minutes at 15±5° C., separated and the organic phase was collected.
13. The organic phase was concentrated until the volume was 7-8V (actual volume was 300 L), while controlling the reactor inner temperature to no more than 40° C. (jacket temperature no more than 45° C.).
14. n-Heptane (10.0 V, 270.60 kg) was charged to the reactor and concentrated until the volume was 7-8V (actual volume was 286 L), while controlling the reactor inner temperature to no more than 40° C. (jacket temperature no more than 45° C.).
15. n-Heptane (10.0 V, 270.80 kg) was charged to the reactor. The mixture was concentrated until the volume was 7˜8V (actual volume was 318 L), controlling the temperature as described above.
16. n-Heptane (10.0 V, 267.15 kg) was charged to the reactor and stirred for at least 30 mins at 25±5° C.
17. The suspension was centrifuged, and the cake was washed with n-heptane (5.0 V, 135.66 kg). The filter cake was collected and transferred to a vacuum oven.
18. The cake was dried under vacuum at 40±5° C., P≤−0.08 MPa for at least 6 hours, turning at least every 2 hours and sampled for LOD until LOD≤5%.
19. (3aS,4S,6aS)-5-(tert-Butoxycarbonyl)-2,2-dimethyl-6-oxotetrahydro-4H-[1,3]dioxolo[4,5-c]pyrrole-4-carboxylic acid was obtained as a solid (24.1 kg, 96.1% purity, 98.1% Q-NMR and 56.6% yield). Stored in double LDPE bags sealed with cable ties, within well closed containers at room temperature.

The above process was repeated using (3aS,4S,6aR)-5-(tert-butoxycarbonyl)-2,2-dimethyltetrahydro-4H-[1,3]dioxolo[4,5-c]pyrrole-4-carboxylic acid (37.5 kg) to provide a second batch of (3aS,4S,6aS)-5-(tert-butoxycarbonyl)-2,2-dimethyl-6-oxotetrahydro-4H-[1,3]dioxolo[4,5-c]pyrrole-4-carboxylic acid (23.5 kg, 97.7% purity, 99.4% Q-NMR and 59.4% yield).

### Step k

1. Acetonitrile (3.5 V, 64.80 kg) was charged to a reactor under nitrogen atmosphere and stirring started.
2. (3aS,4S,6aS)-5-(tert-Butoxycarbonyl)-2,2-dimethyl-6-oxotetrahydro-4H-[1,3]dioxolo[4,5-c]pyrrole-4-carboxylic acid (1.0eq, 23.50 kg) was charged to the reactor.
3. The reactor wall was rinsed with acetonitrile (0.5 V, 9.25 kg) and the temperature was adjusted to 15-20° C.
4. Trifluoroacetic acid (4.0eq, 35.45 kg) was charged dropwise to the reactor (over at least 2 h is recommended).
5. The reaction was stirred for at least 12 hours at 20±5° C., until the assay of (3aS,4S,6aS)-5-(tert-butoxycarbonyl)-2,2-dimethyl-6-oxotetrahydro-4H-[1,3]dioxolo[4,5-c]pyrrole-4-carboxylic acid %, measured by HPLC analysis.
6. Methanol (2.0 V, 37.90 kg) was charged to the reactor at 20±5° C. and stirred for at least 2 h at 20±5° C.
7. MTBE (10.0 V, 171.15 kg) was charged to the reactor at 20±5° C. and stirred for at least 1 h at 20±5° C.
8. The suspension was centrifuged, and the cake was washed with MTBE (10.0 V, 173.68 kg). The filter cake was collected and transferred to a vacuum oven.
9. The cake was dried under vacuum at 35±5° C., P≤−0.08 MPa for at least 6 hours, turning at least every 2 hours and sampled for LOD until LOD≤5%.

The above process was repeated using (3aS,4S,6aS)-5-(tert-butoxycarbonyl)-2,2-dimethyl-6-oxotetrahydro-4H-[1,3]dioxolo[4,5-c]pyrrole-4-carboxylic acid (23.50 kg) and the batches were combined to provide (3aS,4S,6aS)-2,2-dimethyl-6-oxotetrahydro-4H-[1,3]dioxolo[4,5-c]pyrrole-4-carboxylic acid as a white solid (24.3 kg, 99.96% purity and 76.5% yield). Stored in double LDPE bags sealed with cable ties, within well closed containers at room temperature.

### Intermediate 1: (3aS,4S,6aS)-2,2-Dimethyl-6-oxotetrahydro-4H-[1,3]dioxolo[4,5-c]pyrrole-4-carboxylic acid (Alternative Conditions)

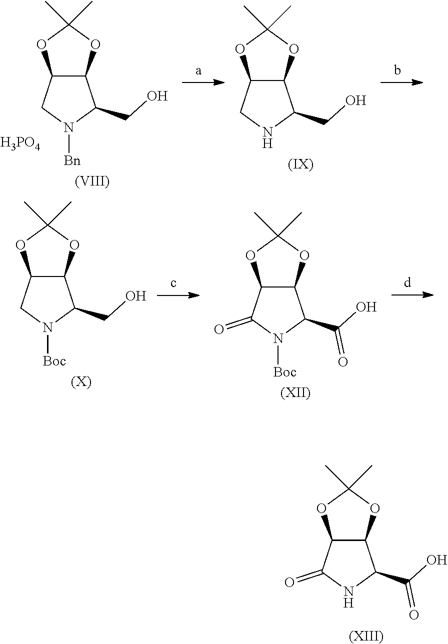

### Steps a, b

1. Methanol (7.00 V, 388.95 kg), ((3aS,4R,6aR)-5-benzyl-2,2-dimethyltetrahydro-4H-[1,3]dioxolo[4,5-c]pyrrol-4-yl)methanol phosphate salt (1.00 eq. 70 kg) and TEA (1.50 eq. 29.45 kg) were charged to a reactor under nitrogen atmosphere.
2. The temperature was adjusted to 25±5° C., the mixture stirred until dissolved.
3. The solution was transferred through an activated carbon filter, the filter was washed with MeOH (2 V, 112.45 kg) and combined.
4. The autoclave was purged 5 times with nitrogen. Each time the pressure was increased to 0.5 MPa and released to 0.1 MPa. The filtered solution was charged to the autoclave.
5. Palladium on carbon (7% w/w, 4.900 kg) was charged to the autoclave, the addition funnel and charging port was rinsed with MeOH (1.00±0.5 V, 28.20 kg).
6. The air in the autoclave was replaced with nitrogen and hydrogen successively, hydrogen was charged to 0.5-0.8 MPa, and the reaction mixture was heated to 60±5° C.
7. The temperature was controlled at 60±5° C. and the pressure at no more than 0.8 MPa, repeat the procedure until the system pressure change is less than 0.1 MPa in one hour.
8. The reaction was stirred for at least 24 hours, until the assay of ((3aS,4R,6aR)-5-benzyl-2,2-dimethyltetrahydro-4H-[1,3]dioxolo[4,5-c]pyrrol-4-yl)methanol ≤2%, measured by HPLC analysis.
9. The autoclave was purged with nitrogen. TEA (2.00 eq. 39.20 kg) and di-tert-butyl dicarbonate (1.20 eq. 50.10 kg) were charged to the reactor at 0-30° C. The reaction mixture was stirred for at least 3 h at 25±5° C. until the assay of ((3aS,4R,6aR)-2,2-dimethyltetrahydro-4H-[1,3]dioxolo[4,5-c]pyrrol-4-yl)methanol ≤1%, measured by HPLC analysis.
10. The reaction mixture was filtered under nitrogen atmosphere. The filter was washed with MeOH (3.00 V, 159.0 kg) and the filtrate was collected.
11. The solution was concentrated until the volume was 4˜5V (actual volume was 315 L), while controlling the reactor inner temperature to no more than 50° C. (jacket temperature no more than 55° C.).
12. Ethyl acetate (10.00 V, 634.45 kg) and water (10.00 V, 705.00 kg) were charged successively to the reactor, the temperature adjusted to 25±5° C. and stirred for at least 15 minutes. The mixture was allowed to stand for at least 30 minutes, separated and the organic phase was collected.
13. Ethyl acetate (10.00 V, 611.60 kg) was charged to the reactor containing the aqueous phase, and stirred for at least 15 minutes at 25±5° C. The mixture was allowed to stand for at least 30 minutes, separated and the organic phase was collected.
14. The organic phases were combined and concentrated until the volume was 4-5V (actual volume was 320 L), while controlling the reactor inner temperature to no more than 50° C. (jacket temperature no more than 55° C.).
15. Ethyl acetate (10.00 V, 632.05 kg) was charged to the reactor and concentrated until the volume was 4-5V (actual volume was 320 L), while controlling the reactor inner temperature to no more than 50° C. (jacket temperature no more than 55° C.).
16. Ethyl acetate (10.00 V, 626.05 kg) was charged to the reactor. The solution was concentrated until the volume was 3˜4V (actual volume was 285 L), controlling the temperature as described above.
17. tert-Butyl (3aS,4R,6aR)-4-(hydroxymethyl)-2,2-dimethyltetrahydro-5H-[1,3]dioxolo[4,5-c]pyrrole-5-carboxylate was collected as an ethyl acetate solution (240 kg, 96.6% purity, 22.2% assay and 100.6% yield) and stored in a drum at room temperature. tert-Butyl (3aS,4R,6aR)-4-(hydroxymethyl)-2,2-dimethyltetrahydro-5H-[1,3]dioxolo[4,5-c]pyrrole-5-carboxylate was used directly after qualified.

The above process was repeated using ((3aS,4R,6aR)-5-benzyl-2,2-dimethyltetrahydro-4H-[1,3]dioxolo[4,5-c]pyrrol-4-Amethanol phosphate salt (70 kg) to provide a second batch of tert-butyl (3aS,4R,6aR)-4-(hydroxymethyl)-2,2-dimethyltetrahydro-5H-[1,3]dioxolo[4,5-c]pyrrole-5-carboxylate ethyl acetate solution (282 kg, 97.6% purity, 18.4% assay and 98.0% yield).

### Step c

1. Water (15.0 V, 763 kg) and ethyl acetate (10.0 V, 458.55 kg) were charged to a reactor under nitrogen atmosphere and stirring started.
2. tert-Butyl (3aS,4R,6aR)-4-(hydroxymethyl)-2,2-dimethyltetrahydro-5H-[1,3]dioxolo[4,5-c]pyrrole-5-carboxylate ethyl acetate solution (1.0eq, 240.00 kg of ethyl acetate solution, the assay content is 53 kg) was charged to the reactor and cooled to 5±5° C.
3. Ruthenium trichloride hydrate (0.05 eq, 2.156 kg) was charged to reactor at 0±5° C. Adjusted the temperature to 15±10° C.
4. Sodium periodate (8.0eq, 336.95 kg) was charged to the reactor in batches (no less than 10 batches) at 15±10° C., allowing at least 10 minutes between batches.
5. The reaction was stirred for at least 40 hours at 25±5° C., until the assay of tert-butyl (3aS,4R,6aR)-4-(hydroxymethyl)-2,2-dimethyltetrahydro-5H-[1,3]dioxolo[4,5-c]pyrrole-5-carboxylate ≤1%, measured by HPLC analysis. Adjusted the temperature to 5-10° C.
6. Methanol (1.0 V, 42.20 kg) and diatomite 50% w/w, 26.70 kg) were charged to the reactor at 15±10° C. and stirred for at least 15 mins at 15±10° C.
7. The mixture was filtered and the cake was washed with ethyl acetate (5.0 V, 218.55 kg).
8. The temperature was controlled at 25±5° C. and stirred for at least 15 minutes. The mixture was allowed to stand for at least 30 minutes, separated and the organic phase was collected.
9. Ethyl acetate (5.0 V, 242.90 kg) was charged to the reactor containing the aqueous phase, and stirred for at least 15 minutes at 25±5° C. The mixture was allowed to stand for at least 30 minutes, separated and the organic phase was collected.
10. Ethyl acetate (5.0 V, 242.40 kg) was charged to the reactor containing the aqueous phase, and stirred for at least 15 minutes at 25±5° C. The mixture was allowed to stand for at least 30 minutes, separated and the organic phase was collected.
11. Ethyl acetate (5.0 V, 242.70 kg) was charged to the reactor containing the aqueous phase, and stirred for at least 15 minutes at 25±5° C. The mixture was allowed to stand for at least 30 minutes, separated and the organic phase was collected.
12. A 1:1 aqueous solution of 16.7% aq. solution of sodium hydrogen sulfite and sodium chloride (3.0 V, 199.45 kg) was charged to the reactor containing the combined organic phases and stirred for at least 30 minutes. The mixture was allowed to stand for at least 30 minutes at 15±5° C., separated and the organic phase was collected.
13. Ethyl acetate (5.0 V, 239.90 kg) was charged to the reactor containing the aqueous phase and stirred for at least 15 mins at 15±5° C. The mixture was allowed to stand for at least 30 minutes, separated and the organic phase was collected.
14. Ethyl acetate (5.0 V, 239.90 kg) was charged to the reactor containing the aqueous phase and stirred for at least 15 mins at 15±5° C. The mixture was allowed to stand for at least 30 minutes, separated and the organic phase was collected.
15. Ethyl acetate (5.0 V, 239.90 kg) was charged to the reactor containing the aqueous phase and stirred for at least 15 mins at 15±5° C. The mixture was allowed to stand for at least 30 minutes, separated and the organic phase was collected.
16. A 10% aq. solution of sodium chloride (1.0 V, 53.75 kg) was charged to the reactor containing the organic phase and stirred for at least 20 minutes at 15±5° C. The mixture was allowed to stand for at least 30 minutes at 15±5° C., separated and the organic phase was collected.
17. The organic phase was concentrated until the volume was 7˜8V (actual volume was 380 L), while controlling the reactor inner temperature to no more than 40° C. (jacket temperature no more than 45° C.).
18. Adjusted the reactor inner temperature to 80±7° C., stirred for at least 1 hour, cooled to 25±5° C.
19. n-Heptane (30.0 V, 1090.38 kg) was charged to the reactor at 25±5° C., cooled to 5±5° C. and stirred for at least 1 hour.
20. The suspension was centrifuged, and the cake was washed with n-heptane (3.0 V, 110.40 kg). The filter cake was collected and transferred to a vacuum oven.
21. The cake was dried under vacuum at 40±5° C., P≤−0.08 MPa for at least 8 hours, turning at least every 2 hours and sampled for LOD until LOD≤5%.
22. (3aS,4S,6aR)-5-(tert-ButoxycarbonyI)-2,2-dimethyltetrahydro-4H-[1,3]dioxolo[4,5-c]pyrrole-4-carboxylic acid was obtained as a solid (23.57 kg, 94.9% purity and 40.4% yield over three steps).

The above process was repeated using tert-butyl (3aS,4R,6aR)-4-(hydroxymethyl)-2,2-dimethyltetrahydro-5H-[1,3]dioxolo[4,5-c]pyrrole-5-carboxylate ethyl acetate solution (282 kg of ethyl acetate solution, the assay content 52 kg) to provide a second batch of (3aS,4S,6aR)-5-(tert-butoxycarbonyI)-2,2-dimethyltetrahydro-4H-[1,3]dioxolo[4,5-c]pyrrole-4-carboxylic acid (26.55 kg, 92.4% purity and 44.7% yield over three steps).

### Step d

1. Acetonitrile (5.5 V, 215.35 kg) was charged to a reactor under nitrogen atmosphere and stirring started.
2. (3aS,4S,6aS)-5-(tert-Butoxycarbonyl)-2,2-dimethyl-6-oxotetrahydro-4H-[1,3]dioxolo[4,5-c]pyrrole-4-carboxylic acid (1.0eq, 49.95 kg) was charged to the reactor.
3. The reactor wall was rinsed with acetonitrile (0.5 V, 20.00 kg) and the temperature was adjusted to 20±5° C.
4. Trifluoroacetic acid (4.0eq, 75.15 kg) was charged dropwise to the reactor (over at least 2 hours is recommended).
5. The reaction was stirred for at least 30 hours at 20±5° C., until the assay of (3aS,4S,6aS)-5-(tert-butoxycarbonyl)-2,2-dimethyl-6-oxotetrahydro-4H-[1,3]dioxolo[4,5-c]pyrrole-4-carboxylic acid %, measured by H PLC analysis.
6. Methanol (2.0 V, 79.60 kg) was charged to the reactor at 20±5° C. and stirred for at least 2 hours at 20±5° C.
7. MTBE (30.0 V, 1115.70 kg) was charged to the reactor at 20±5° C. and concentrated until the volume was 15˜20V (actual volume was 950 L), while controlling the reactor inner temperature to no more than 30° C. (jacket temperature no more than 35° C.).
8. MTBE (20.0 V, 735.05 kg) was charged to the reactor and concentrated until the volume was 15˜20V (actual volume was 810 L), while controlling the reactor inner temperature to no more than 30° C. (jacket temperature no more than 35° C.).
9. MTBE (20.0 V, 739.55 kg) was charged to the reactor and concentrated until the volume was 15˜20V (actual volume was 790 L), while controlling the reactor inner temperature to no more than 30° C. (jacket temperature no more than 35° C.).
10. MTBE (20.0 V, 745.25 kg) was charged to the reactor and stirred for at least 1 hour at 20±5° C.
11. The suspension was centrifuged, and the cake was washed with MTBE (10.0 V, 369.5 kg). The filter cake was collected and transferred to a vacuum oven.
12. The cake was dried under vacuum at 35±5° C., P≤−0.08 MPa for at least 6 hours, turning at least every 2 hours and sampled for LOD until LOD≤5%. Provided (3aS,4S,6aS)-2,2-dimethyl-6-oxotetrahydro-4H-[1,3]dioxolo[4,5-c]pyrrole-4-carboxylic acid as a white solid (31.11 kg, 99.9% purity and 93.3% yield). Stored in double LDPE bags sealed with cable ties, within well closed containers at room temperature.

### Intermediate 2: 5-Chloro-2,4-difluoro-N-(methyl-d3)aniline hydrochloride

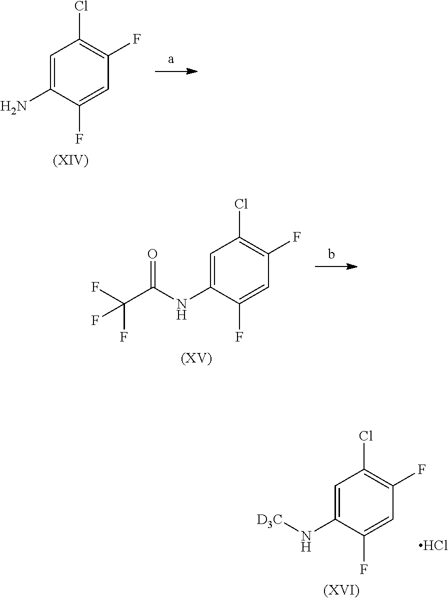

### Step a

1. DMF (5.0 V, 180 L) was charged to a 500 L reactor with mechanical stirrer and stirring started.
2. 5-Chloro-2,4-difluoroaniline (1.0 eq, 36.0 kg) was charged in portions to the reactor at 18° C. and stirred for at least 30 minutes until fully dissolved.
3. The solution was cooled to 5° C.
4. Trifluoroacetic anhydride (1.0 eq, 55.5 kg) was charged slowly to the reactor at 5° C.
5. The reaction temperature increased 18° C. and the reaction was stirred for 12 hours.
5. The reaction mixture was poured into water (15.0 V, 540 L) and stirred for 2 hours.
6. The suspension was filtered, and the collected solid was triturated with water (4.2 V, 150 L).
7. The aq. suspension was centrifuged, the cake was collected and dried in an oven at 50° C. for 24 hours to give N-(5-chloro-2,4-difluorophenyI)-2,2,2-trifluoroacetamide as an off-white solid (49.0 kg, 99.8% purity and 85.8% yield).

### Step b

1. DMF (5.0 V, 210 L) was charged to a 500 L reactor with mechanical stirrer and stirring started.
2. N-(5-Chloro-2,4-difluorophenyl)-2,2,2-trifluoroacetamide (1.0 eq, 43.0 Kg) was charged to the reactor at 18° C.
3. The solution was cooled to 10° C.
4. Pre-milled potassium carbonate (1.5 eq, 34.3 kg) was charged to the reactor at 10° C.
5. Methyl-d3 iodide (1.15 eq, 27.6 kg) was charged to the reactor at 10° C. and the temperature was raised to 18° C. after the addition.
6. The reaction was stirred for 30 minutes at 18° C. and then stirred for 12 hours at 21° C.
7. Potassium acetate (0.5 eq, 8.13 kg) was charged to the reactor.
8. The reaction was stirred for 3 hours at 21° C.
9. Potassium carbonate (1.0 eq, 22.9 kg) at 21° C., over 30 minutes.
10. Water (5.0 V, 210 L) was charged to the reactor at 21° C. and the mixture was stirred for 12 hours.
11. Water (400 L) and n-heptane (150 L) were charged to the reactor and stirred for at least 15 minutes. The mixture was allowed to stand for at least 30 minutes, separated and the organic phase was collected.
12. n-Heptane (150 L) was charged to the reactor containing the aqueous phase and stirred for at least 15 minutes. The mixture was allowed to stand for at least 30 minutes, separated and the organic phase was collected.
13. n-Heptane (150 L) was charged to the reactor containing the aqueous phase and stirred for at least 15 minutes. The mixture was allowed to stand for at least 30 minutes, separated and the organic phase was collected.
14. A saturated aq. solution of sodium chloride (100 L) was charged to the reactor containing the combined organic phases and stirred for at least 15 minutes. The mixture was allowed to stand for at least 30 minutes, separated and the organic phase was collected.
15. A saturated aq. solution of sodium chloride (100 L) was charged to the reactor containing the n-heptane solution and stirred for at least 15 minutes. The mixture was allowed to stand for at least 30 minutes, separated and the organic phase was collected, dried over sodium sulfate and filtered.
16. The n-heptane solution (450 L) was charged to a 1000 L reactor and cooled to 0° C.
17. HCl gas was bubbled through the mixture at 0-5° C. for 7 hours.
18. The suspension was filtered and the cake was washed with n-heptane (2x 50 L).
19. The filter cake was collected and dried in an oven to give 5-chloro-2,4-difluoro-N-(methyl-d3)aniline hydrochloride as a white solid (28.0 kg, 99.5% purity and 84.5% yield).

### Intermediate 3: 2-Bromo-6-methyl-4-(trifluoromethyl)pyridine

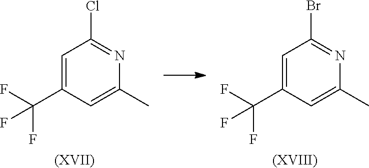 [0522] 1. Five reactions were carried out in parallel.
2. Acetonitrile (2.5 V, 12.5 L) was charged to a 50 L reactor with mechanical stirrer and stirring started.
3. 2-Chloro-6-methyl-4-(trifluoromethyl)pyridine (CAS Number 22123-14-4; 5.00 kg) was charged to the reactor at 15-20° C.

4. Trimethylsilyl bromide (7.83 kg) was carefully charged to the reactor in batches (rate 500 g/min) at 15-20° C.
5. The reaction mixture was heated to 72-75° C. (jacket temperature no more than 85° C.) and stirred for 10 hours.
6. The reaction mixture was distilled to about 5.0 L. Fresh acetonitrile (2.0 V, 10.0 L) was added to the residue and then distilled to about 5.0 L again.
7. Acetonitrile (2.5 V, 12.5 L) was charged to the reactor at 40-45° C.
8. Trimethylsilyl bromide (7.05 kg) was carefully charged to the reactor in batches (rate 500 g/min) at 40-45° C.
9. The reaction mixture was heated to 72-75° C. (jacket temperature no more than 85° C.) and stirred for 16 hours.
10. The combined acetonitrile solutions (five batches) were distilled to provide a crude oil (29.3 L), while controlling the temperature to no more than 45° C.
11. The crude oil was directly slowly transferred (rate 1 kg/min) into a 50 L reactor containing water (17.0 L) at 0-5° C.
12. A saturated aq. sodium hydrogen carbonate (3.00 L) was charged to the reactor to adjust the pH to 6-7 and stirred for ˜15 minutes. The mixture was allowed to stand for at least 30 minutes at 25° C., separated and the lower organic layer was collected.
13. A saturated aq. solution of sodium chloride (7.0 L) was charged into the reactor 5 containing the organic phase and stirred for at least 15 minutes. The mixture was allowed to stand for at least 30 minutes, separated and the lower organic layer was collected.
14. A saturated aq. solution of sodium chloride (7.0 L) was charged to the reactor containing the organic phase and stirred for at least 15 minutes. The mixture was allowed to stand for at least 30 minutes, separated and the lower organic layer was collected.
15. The oil was dried over sodium sulfate and filtered to provide the crude product (25.1 kg) as a yellow oil.
16. The oil was purified via distillation at 122° C., 0.09 MPa (water pump) to give 2-bromo-6-methyl-4-(trifluoromethyl)pyridine as a colourless oil (23.8 kg, 99.3% purity and 75.0% yield).

### Example 1: (2S,3S,4S)-N-(5-Chloro-2,4-difluorophenyl)-3,4-dihydroxy-N-(methyl-d3)-1-(6-methyl-4-(trifluoromethyl)pyridin-2-yl)-5-oxopyrrolidine-2-carboxamide (Form A)

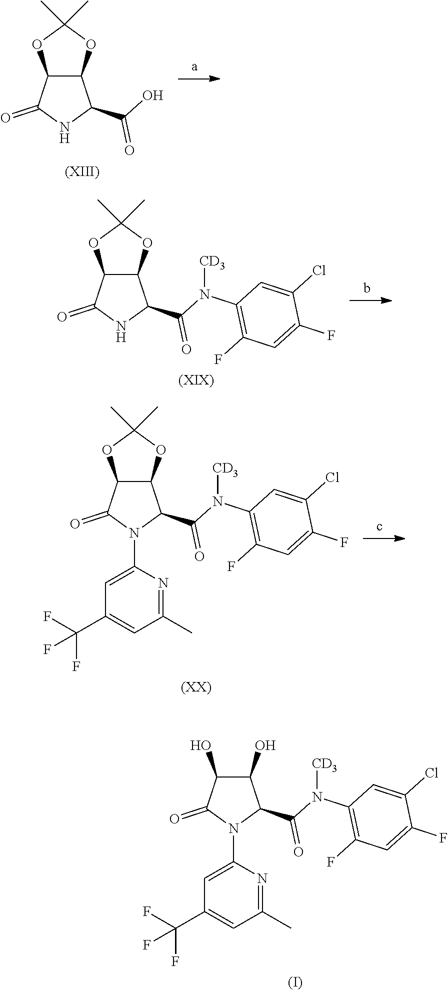

### Step a

1. A 1000 L vessel was rinsed with acetonitrile (20.3 kg) and dried under vacuum. To the vessel was charged Intermediate 1 (1.0 eq., 16.7 kg), Intermediate 2 (1.0 eq., 18.0 kg), acetonitrile (10 V, 131.2 kg) and pyridine (1.0 eq., 6.6 kg), followed by a 50% solution of 1-propanephosphonic anhydride in ethyl acetate (2.5 eq., 132.1 kg). Ethyl acetate (10.8 kg) was used as a line rinse. After inerting the vessel with partial vacuum, the contents of the reactor were aged at 30° C. for 21 h.
2. Ethyl acetate (10 V, 140.2 kg) was charged to the reactor vessel and the contents of the reaction were cooled to 10° C. The reaction was quenched with 10% sodium chloride solution (15 V, 251 kg, prepared by dissolving sodium chloride (25.1 kg) in purified water (225.8 kg)), while maintaining the temperature of the reaction below 20° C. After quenching, the reaction was stirred for 30 min at 20° C., allowed to stand, the layers separated and the organic phase was collected.
3. A 10% solution of potassium phosphate tribasic (10 V, 166.5 kg, taken from a solution prepared by dissolving potassium phosphate tribasic (36.7 kg) in purified water (330.5 kg) portion-wise while maintaining the temperature at below 30° C.) was charged to the reactor with the organic phase and agitated for 5 minutes. The mixture was allowed to stand, the layers separated and the bottom aqueous phase was removed.
4. A 10% solution of potassium phosphate tribasic (10 V, 166.5 kg) was charged to the reactor with the organic phase and agitated for 5 minutes. The mixture was allowed to stand, the layers separated and the bottom aqueous phase was removed.
5. n-Heptane (3 V, 34.3 kg) was charged to the reactor containing the organic phase followed by 20% citric acid solution (5 V, 83.3 kg, prepared by dissolving citric acid (16.7 kg) in purified water (66.6 kg)) and agitated for 5 minutes. The mixture was allowed to stand, the layers separated and the bottom aqueous phase was removed.
6 The organic layer was transferred from the 1000 L vessel into plastic lined drums.
7. The aqueous washes were combined in the 1000 L vessel and back-extracted with a mixture of ethyl acetate (5 V, 76.5 kg) and n-heptane (1 V, 11.6 kg). The layers were agitated for 5 minutes. The mixture was allowed to stand, the layers separated and the bottom aqueous phase was removed.
8. To the back-extracted organic layer was charged the remaining 10% solution of potassium phosphate tribasic (2 V, 34.8 kg). The layers were agitated for 5 minutes. The mixture was allowed to stand, the layers separated and the organic phase was collected.
9. The combined organic extracts were charged into a 400 L vessel via a 1 micron inline filter. The solution was concentrated under reduced pressure until the volume was 3 V (approx. 90 L), maintaining the internal temperature at <40° C.
10. Acetonitrile (7 V, 168.0 kg) was charged to the reactor and solution was concentrated under reduced pressure until the volume was 3 V (approx. 90 L), maintaining the internal temperature at <40° C.
11. Acetonitrile (6 V, 147.0 kg) was charged to the reactor and solution was concentrated under reduced pressure until the volume was 3 V (approx. 90 L), maintaining the internal temperature at <40° C.
12. Ethanol (6.5 V, 157.5 kg) was charged to the reactor and solution was distilled under reduced pressure until 114 L had been removed. A second portion of ethanol (5.5 V, 129.0 kg) was charged, and solution was concentrated under reduced pressure until the volume was 3 V (approx. 90 L), maintaining the internal temperature at <40° C.
13. The solution was cooled to 25° C. and the slurry was aged for 25 hours at 20° C.

14. n-Heptane (3 V, 62.1 kg) was charged and the crystallisation solution was cooled to 0° C. and aged for 1 hour.
15. The slurry was filtered, and the vessel and wet cake were washed with a mixture of n-heptane (1.0 V, 21.5 kg) and ethanol (1.0 V, 23.2 kg). The wet cake was dried under a 10 nitrogen flow for 90 min, transferred to trays and dried under vacuum at 50° C.
16. (3aS,4S,6aS)-N-(5-Chloro-2,4-difluorophenyI)-2,2-dimethyl-N-(methyl-d3)-6-oxotetrahydro-4H-[1,3]dioxolo[4,5-c]pyrrole-4-carboxamide was obtained as a white solid (26.35 kg, 99.5% purity and 87% yield). The dried solid was transferred to double-bagged polythene liners placed within HDPE drums.

### Step b

1. A 400 L vessel was rinsed with N,N-dimethylacetamide (20.2 kg). To the vessel was charged (3aS,4S,6aS)-N-(5-chloro-2,4-difluorophenyI)-2,2-dimethyl-N-(methyl-d3)-6-oxotetrahydro-4H-[1,3]dioxolo[4,5-c]pyrrole-4-carboxamide (1.0 eq., 22.0 kg) and the vessel was inerted with nitrogen.
2. N,N-Dimethylacetamide (2 V, 41.8 kg) was charged to the reactor vessel, followed by toluene (2 V, 38.1 kg) and water (1.0 eq., 1.09 kg). The contents of the reactor were adjusted to 20° C. and the mixture was aged until a solution had formed.
3. Potassium carbonate (1.5 eq., 12.60 kg) and copper(I) iodide (0.1 eq., 1.15 kg) were charged to a visually dry 1000 L vessel and the vessel was inerted with nitrogen. Toluene (5 V, 95.1 kg) was charged and the vessel was re-inerted via a pressure purge.
4. N,N′-Dimethylethylenediamine (0.2 eq., 1.07 kg) was charged to the contents of the 1000 L vessel, followed by Intermediate 3 (1.1 eq., 16.73 kg), using toluene (2.0 kg) as a line-rinse. The contents of the vessel were warmed to 40° C.
5. The solution of (3aS,4S,6aS)-N-(5-chloro-2,4-difluorophenyI)-2,2-dimethyl-N-(methyl-d3)-6-oxotetrahydro-4H-[1,3]dioxolo[4,5-c]pyrrole-4-carboxamide in the 400 L vessel was transferred to the slurry in the 1000 L vessel using a slight positive pressure of nitrogen, over a period of 1 hour.
6. N,N-Dimethylacetamide (0.5 V, 10.4 kg) and toluene (0.5 V, 9.5 kg) were charged to the 400 L vessel as a rinse, and this rinse was then transferred to the 1000 L vessel.
7. The contents of the 1000 L vessel were aged for 3 hours at 40° C. with the stir speed set to 90 rpm.
8. The contents of the 1000 L vessel were cooled to <25° C. and 20% ammonium chloride solution (10 V, 220.0 kg, taken from a solution prepared by dissolving ammonium chloride (88.0 kg) in purified water (352.0 kg)) was charged to the vessel.
9. Isopropyl acetate (7 V, 134.0 kg) was charged to the vessel and the mixture was agitated for 15 minutes. The mixture was allowed to stand, the layers separated and the bottom aqueous phase was removed.
10. A second portion of 20% ammonium chloride solution (10 V, 220.0 kg) was charged to the organic solution and the mixture was agitated for 5 minutes. The mixture was allowed to stand, the layers separated and the bottom aqueous phase was removed.
11. Water (5 V, 100.0 kg) was charged to the organic solution and the mixture was agitated for 9 minutes. The mixture was allowed to stand, the layers separated and the bottom aqueous phase was removed.
12. The organic layer was transferred to a cleaned 400 L vessel via a 1 micron in-line filter cartridge. The solution was distilled under reduced pressure whilst maintaining the internal temperature at <55° C. until the volume was approx. 110 L.
13. Toluene (10 V, 190.7 kg) was charged to the 1000 L vessel, to rinse the vessel, discharged into drums and then transferred to the 400 L vessel via the same 1 micron in-line filter cartridge as used previously.
14. The solution in the 400 L vessel was distilled under reduced pressure whilst maintaining the internal temperature at <55° C. until the volume was approx. 110 L.
15. The contents of the 400 L vessel were cooled to 40.5° C., a sample was taken for analysis and the slurry was further cooled to 20° C. before aging for >12 hours.
18. n-Heptane (10 V, 150.6 kg) was charged to the vessel over a period of 1 hour and the batch was then aged for 1 hour.
19. The slurry was filtered (through a large oyster filter), and the vessel and wet cake was washed with a mixture of n-heptane (2 V, 30.1 kg) and toluene (1.0 V, 19.4 kg). The cake was further washed with n-heptane (5 V, 75.2 kg)
20. The wet cake was dried under a nitrogen flow for 1 hour, transferred to trays and dried at 50° C. with a nitrogen sweep.
21. (3aS,4S,6aS)-N-(5-Chloro-2,4-difluorophenyl)-2,2-dimethyl-N-(methyl-d3)-5-(6-methyl-4-(trifluoromethyl)pyridin-2-yl)-6-oxotetrahydro-4H-[1,3]dioxolo[4,5-c]pyrrole-4-carboxamide was obtained as a white solid (27.74 kg, 97.9% purity and 86% corrected isolated yield). The dried solid was transferred to double-bagged polythene liners placed within HDPE drums.

### Step c

1. (3aS,4S,6aS)-N-(5-Chloro-2,4-difluorophenyl)-2,2-dimethyl-N-(methyl-d3)-5-(6-methyl-4-(trifluoromethyl)pyridin-2-yl)-6-oxotetrahydro-4H-[1,3]dioxolo[4,5-c]pyrrole-4-carboxamide (1.0 eq., 23.019 kg) and meso-erythritol (2.0 eq., 10.962 kg) were charged to a dry and inert 1000 L vessel. Acetonitrile (6 V, 108.0 kg) was charged to the vessel and the contents were warmed to 40° C.
2. Boron trifluoride diethyl etherate (3.0 eq., 18.645 kg) was charged to the vessel at 40° C. via a dosing pump, with acetonitrile (1.012 kg) used as a line rinse. The reaction was aged for 4 h at 40° C.
3. The reaction mixture was cooled to <25° C. and then aged for 16 hours at 20° C.
4. Isopropyl acetate (8 V, 160.3 kg) was charged to the reaction mixture, followed by 1 M sodium hydroxide (8 V, 188.4 kg, prepared by dissolving 46-51% sodium hydroxide (14.6 kg) in purified water (173.8 kg)) and agitated for 5 minutes. The mixture was allowed to stand, the layers separated and the bottom aqueous phase was removed.
5. Water (5 V, 115 kg) was charged to the reactor with the organic phase, followed by 2 M hydrochloric acid (5 V, 118.7 kg, prepared by dissolving 37% hydrochloric acid (22.7 kg) in purified water (96.0 kg)) and agitated for 90 minutes. The mixture was allowed to stand, the layers separated and the organic phase was collected.
6. Water (5 V, 115 kg) was charged to the reactor with the organic phase, followed by 2 M hydrochloric acid (5 V, 118.7 kg, prepared as described above) and agitated for 90 minutes. The mixture was allowed to stand, the layers separated and the bottom aqueous phase was removed.
7. Isopropyl acetate (4 V, 80.7 kg) was charged to the reactor with the organic phase, followed by 7.6 w% aqueous sodium bicarbonate solution (8.0 V, 183.87 kg, prepared by dissolving sodium bicarbonate (13.87 kg) in purified water (170.0 kg) and agitated for 5 minutes. The mixture was allowed to stand, the layers separated and the organic phase was collected.
8. Water (4 V, 92.1 kg) was charged to the reactor with the organic phase and agitated for 90 minutes. The mixture was allowed to stand, the layers separated and the organic phase was collected into drums.
9. The organic solution was transferred to a 400 L vessel through a 1 pm inline filter.
10. The solution was concentrated under reduced pressure until the volume was 4 V (approx. 85 L), maintaining the internal temperature at <40° C.
11. Isopropyl acetate (4 V, 74.1 kg) was charged to the vessel and concentrated under reduced pressure until the volume was 4 V (approx. 85 L), maintaining the internal temperature at <40° C.
12. The solution was warmed to 45° C. and (2S,3S,4S)-N-(5-chloro-2,4-difluorophenyl)-3,4-dihydroxy-N-(methyl-d3)-1-(6-methyl-4-(trifluoromethyl)pyridin-2-yl)-5-oxopyrrolidine-2-carboxamide Form A seed (0.54 w %, 0.115 kg) was charged using a PuroVaso container via the 4″ ball valve. The solution was aged for 4 min, and then n-heptane (4 V, 58.1 kg) was slowly charged over approx. 50 min.
13. The stirred batch was aged for 1 hour at 45° C. during which a visible seed bed developed. The slurry was slowly cooled to 40° C. over 6 hours and then aged for approx. 8 hours at 40° C. The batch was then cooled to 20° C. over 3 hours.
14. n-Heptane (2 V, 29.0 kg) was charged and the slurry was aged for 1 hour at 20° C.
17. The slurry was filtered, and the vessel was washed with a mixture of n-heptane (1.2 V, 17.3 kg) and isopropyl acetate (0.8 V, 14.6 kg) and this rinse was used to wash the filter-cake. The filter-cake was further washed with n-heptane (1 V, 14.5 kg) and then dried under a nitrogen flow for 1 hour. The solid was transferred to trays and dried under vacuum at 50° C. for 62 hours.
18. (2S,3S,4S)-N-(5-Chloro-2,4-difluorophenyI)-3,4-dihydroxy-N-(methyl-d3)-1-(6-methyl-4-(trifluoromethyl)pyridin-2-yl)-5-oxopyrrolidine-2-carboxamide was obtained as a white solid (17.91 kg, 99.7% purity and 84% yield). The dried solid was transferred to double-bagged polythene liners placed within HDPE drums.

### Example 2: (2S,3S,4S)-N-(5-Chloro-2,4-difluorophenyI)-3,4-dihydroxy-N-(methyl-d3)-1-(6-methyl-4-(trifluoromethyl)pyridin-2-yl)-5-oxopyrrolidine-2-carboxamide hemihydrate Form B)

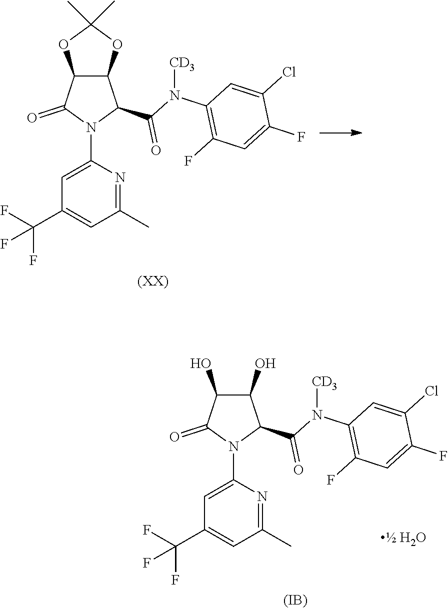 [0591] 1. (3aS,4S,6aS)-N-(5-Chloro-2,4-difluorophenyI)-2,2-dimethyl-N-(methyl-d3)-5-(6-methyl-4-(trifluoromethyl)pyridin-2-yl)-6-oxotetrahydro-4H-[1,3]dioxolo[4,5-c]pyrrole-4-carboxamide (1.0 eq., 2.53 kg) and dichloromethane (20 V, 50.6 L) were charged to a dry and inert 4-necked 100 L vessel under nitrogen atmosphere and the solution was cooled to between −20 and −15° C.
2. A 1 M solution of boron trichloride in dichloromethane (2.4 eq., 11.6 L) was charged drop-wise to the vessel over 2 hours, maintaining the internal temperature at between -20 and −15° C.
3. Upon complete addition, the reaction mixture was warmed to 20° C. and stirred for 1 hour, until the (3aS,4S,6aS)-N-(5-Chloro-2,4-difluorophenyI)-2,2-dimethyl-N-(methyl-d3)-5-(6-methyl-4-(trifluoromethyl)pyridin-2-yl)-6-oxotetrahydro-4H-[1,3]dioxolo[4,5-c]pyrrole-4-carboxamide ≤1%, measured by HPLC analysis.
4. The reaction was quenched by transferring the solution into a 150 L vessel containing 10 w % sodium bicarbonate solution in ice water (20 V, 50.6 L) at 0-5° C.
5. Dichloromethane (5 V, 12.6 kg) was charged to the reactor, the mixture was agitated, the layers allowed to settle and the organic phase was collected.
6. Dichloromethane (5 V, 12.6 kg) was charged to the reactor with the aqueous phase, the mixture was agitated, the layers allowed to settle and the organic phase was collected.
7. Dichloromethane (5 V, 12.6 kg) was charged to the reactor with the aqueous phase, the mixture was agitated, the layers allowed to settle and the organic phase was collected.
8. The combined organic solution was concentrated under reduced pressure to provide crude product (2.6 kg) which was purified by Prep HPLC with the following conditions; column: C18 column; A=acetonitrile; B =water (0.1% ammonium bicarbonate); isocratic gradient 30% A increasing to 60% A over 40 min; detector 210 nm.
9. (2S,3S,4S)-N-(5-Chloro-2,4-difluorophenyl)-3,4-dihydroxy-N-(methyl-d3)-1-(6-methyl-4-(trifluoromethyl)pyridin-2-yl)-5-oxopyrrolidine-2-carboxamide was obtained as a pale yellow solid (2.10 kg and 85% yield).
10. The HPLC purified product (1.90 kg) was charged to a 50 L 4-necked vessel with water (10 V, 19.9 L) and ethanol (5 V, 9.95 L) and the mixture was stirred for 1 hour.
11. (2 S, 3S,4S)-N-(5-Chloro-2,4-difl uorophenyl)-3,4-dihydroxy-N-(methyl-d3)-1-(6-methyl-4-(trifluoromethyl)pyridin-2-yI)-5-oxopyrrolidine-2-carboxamide Form B seed (0.105 kg) was charged to the solution.
12. The solution was stirred at between 15 and 20° C. for 4 days.
13. The slurry was filtered, and the filter-cake was washed with water (1 V x3, 5.97 L). The solid was transferred to trays and dried under vacuum at ambient temperature for 48 hours.
14. (2 S, 3S,4S)-N-(5-Chloro-2,4-difl uorophenyl)-3,4-dihydroxy-N-(methyl-d3)-1-(6-methyl-4-(trifluoromethyl)pyridin-2-yl)-5-oxopyrrolidine-2-carboxamide was obtained as a pale yellow solid (1.85 kg and 98.4% purity).

¹H NMR (300 MHz, Methanol-d4) δ 8.41 (d, J=7.5 Hz, 1H), δ 8.05 (d, J=7.5 Hz, 0.4 H), 7.91 (t, J=7.8 Hz, 0.58H), 7.51 (q, J=9.1 Hz, 1H), 7.36-7.18 (m, 1H), 5.22 (d, J=5.6 Hz, 0.4 H), 5.05 (d, J=5.6 Hz, 0.6 H), 4.32-4.21 (dt, J=22.7, 6.2 Hz, 2H), 2.69 (s, 1H), 2.56 (d, J=10.7 Hz, 2H).

1. A process for preparing a compound of formula (I): 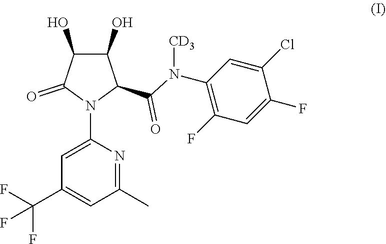 [SMILES: [2H]C([2H])([2H])N(C(=O)[C@@H]1[C@H](O)[C@H](O)C(=O)N1c1cc(C(F)(F)F)cc(C)n1)c1cc(Cl)c(F)cc1F]
which comprises treating a compound of formula (XX): 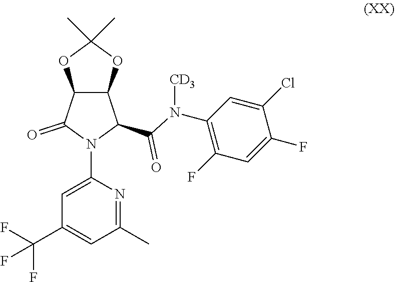 [SMILES: [2H]C([2H])([2H])N(C(=O)[C@@H]1[C@@H]2OC(C)(C)O[C@@H]2C(=O)N1c1cc(C(F)(F)F)cc(C)n1)c1cc(Cl)c(F)cc1F]
with a Lewis acid in the presence of a scavenger agent.

2. The process according to claim 1, wherein the scavenger agent is a diol containing moiety.

3. The process according to claim 2, wherein the diol containing moiety is selected from ethylene glycol, glycerol, 2,3-butanediol or meso-erythritol, in particular meso-erythritol.

4. The process according to any one of claims 1 to 3, wherein the Lewis acid is boron trifluoride (BF 3), such as boron trifluoride diethyl etherate.

5. A process for preparing a compound of formula (I): 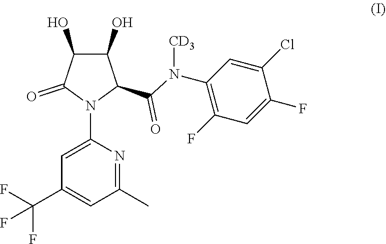 [SMILES: [2H]C([2H])([2H])N(C(=O)[C@@H]1[C@H](O)[C@H](O)C(=O)N1c1cc(C(F)(F)F)cc(C)n1)c1cc(Cl)c(F)cc1F]
which comprises the following steps:

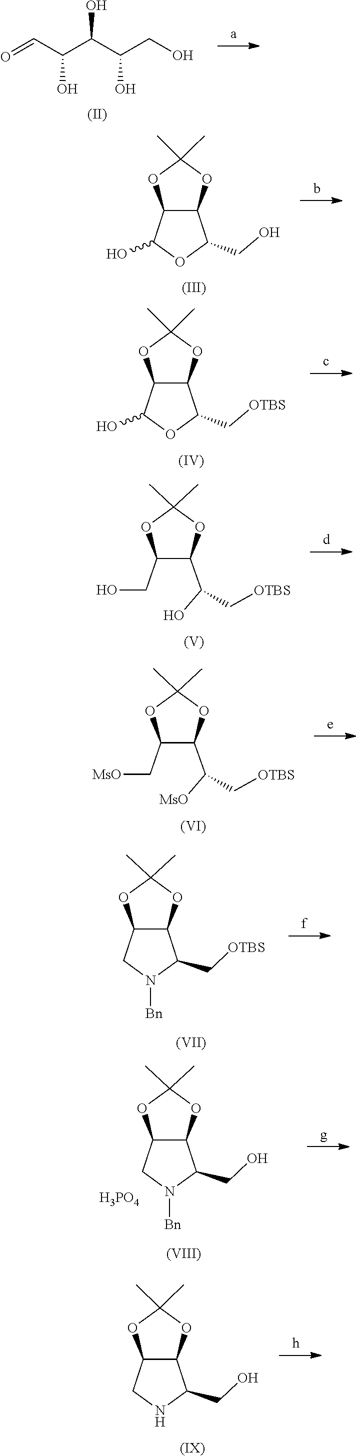

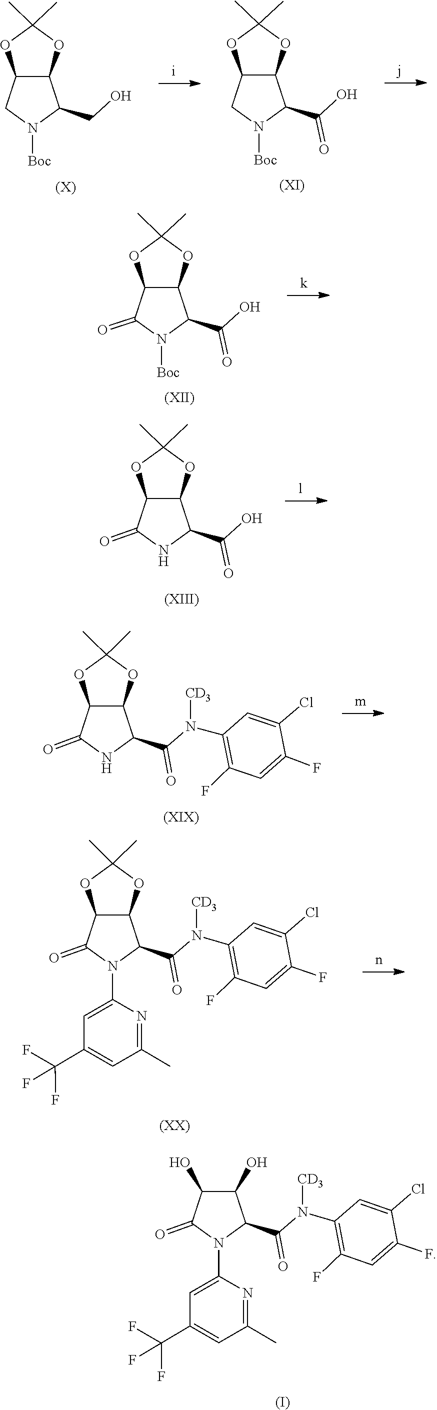

6. A process for preparing a hemihydrate compound of formula (I), i.e. a compound of formula (IB): 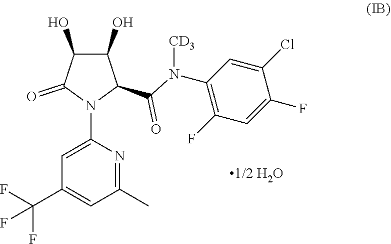 [SMILES: [2H]C([2H])([2H])N(C(=O)[C@@H]1[C@H](O)[C@H](O)C(=O)N1c1cc(C(F)(F)F)cc(C)n1)c1cc(Cl)c(F)cc1F]
which comprises the following steps:

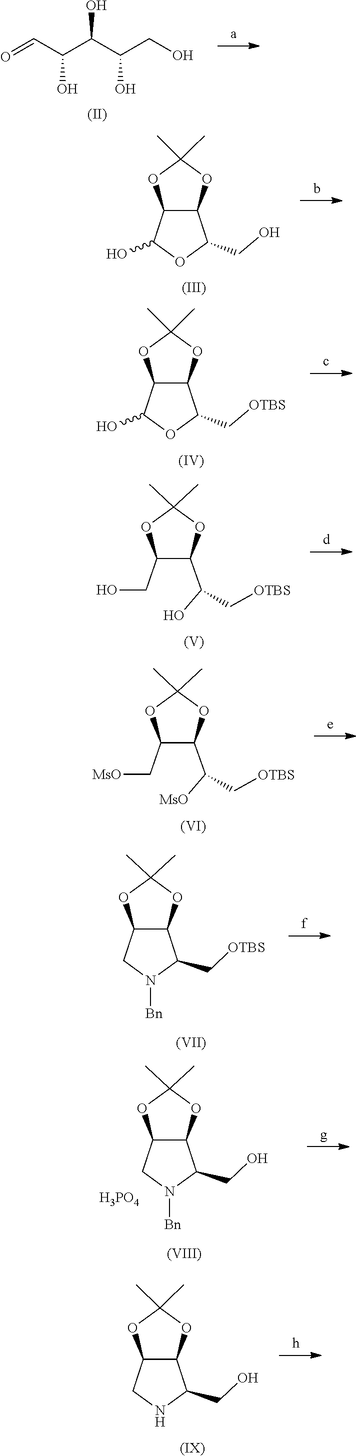

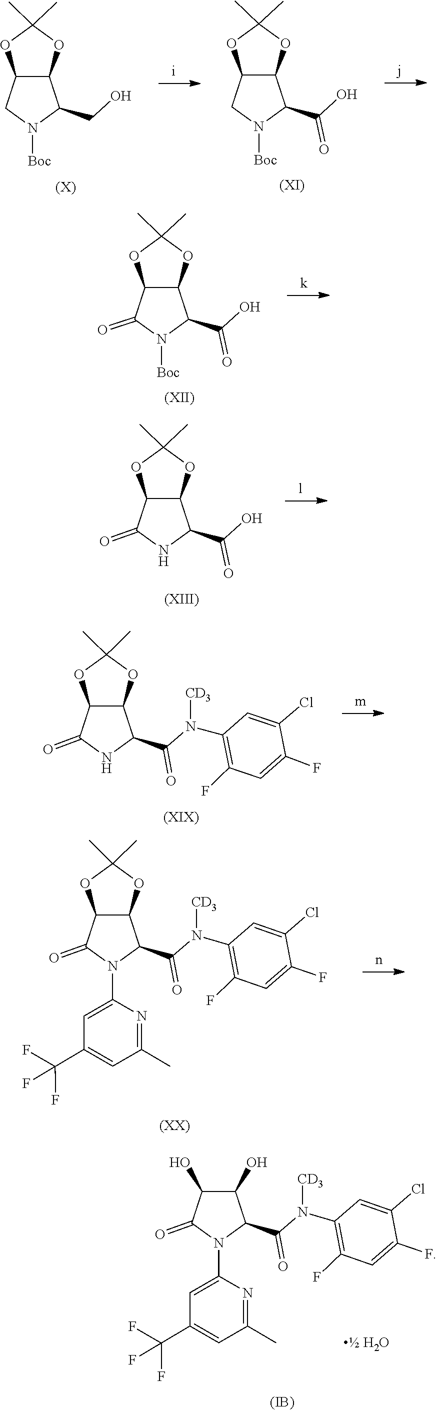

7. A compound of formula (I) obtainable from the process as defined in any one of claims 1 to 6.

8. The compound of formula (I) according to claim 7, which is (2S,3S,4S)-N-(5-Chloro-2,4-difluorophenyl)-3,4-dihydroxy-N-(methyl-d3)-1-(6-methyl-4-(trifluoromethyppyridin-2-yl)-5-oxopyrrolidine-2-carboxamide (Form A) (Example 1).

9. The compound of formula (I) according to claim 8, which is characterised by any one or more of the following parameters:
(i) an XRPD pattern substantially as shown in FIG. 1;
(ii) peaks at the same diffraction angles (2θ) of the XRPD pattern shown in FIGS. 1 and optionally wherein the peaks have the same relative intensity as the peaks shown in FIG. 1;
(iii) major peaks at diffraction angles (2θ) and intensities as those shown in the XRPD pattern in FIG. 1;
(iv) an XRPD pattern having peaks at 6.9±0.5°, 7.6±0.5°, 9.5±0.5°, 11.4±0.5°, 13.7±0.5°, 20.1±0.5°, 20.7±0.5° and 22.6 (20, 1d.p);
(v) an XRPD pattern having peaks at 6.9±0.2°, 7.6±0.2°, 9.5±0.2°, 11.4±0.2°, 13.7±0.2°, 20.1±0.2°, 20.7±0.2° and 22.6±0.2° (20, 1d.p);
(vi) an XRPD pattern having peaks at 6.9±0.1°, 7.6±0.1°, 9.5±0.1°, 11.4±0.1°, 13.7±0.1°, 20.1±0.1°, 20.7±0.1° and 22.6±0.1° (20, 1 d.p);
(vii) an XRPD pattern having peaks at 6.9, 7.6, 9.5, 11.4, 13.7, 20.1, 20.7 and 22.6 (20, 1 d.p);
(viii) an XRPD pattern having peaks as set out in the below table:

| | Relative intensity, |
|---|---|
| Angle, °2θ | %* |
| 6.9 | 94.7 |
| 7.6 | 24 |
| 9.5 | 62.5 |
| 11.4 | 100 |
| 13.7 | 38 |
| 20.1 | 51.4 |
| 20.7 | 23.1 |
| 22.6 | 27.4 |

*Peaks with relative intensity of less than 20% are not reported;
(ix) a differential scanning calorimetry (DSC) onset temperature of 182.26° C.±0.5° C. (such as 182.26° C.±0.2° C., in particular 182.26° C.±0.1° C., more particularly 182.26° C.);
(x) a differential scanning calorimetry (DSC) peak temperature of 182.54° C.±0.5° C. (such as 182.54° C.±0.2° C., in particular 182.54° C.±0.1° C., more particularly 182.54° C.);
(xi) a differential scanning calorimetry (DSC) thermogram as depicted in FIG. 2;
(xii) a thermogravimetric peak mass loss at a temperature of 231.7° C.±0.5° C. (such as 231.7° C.±0.2° C., in particular 231.7° C.±0.1° C., more particularly 231.7° C.); and/or
(xiii) a thermogravimetric analysis (TGA) thermogram as depicted in FIG. 3.

10. The compound of formula (I) according to claim 7, which is (2S,3S,4S)-N-(5-Chloro-2,4-difluorophenyl)-3,4-dihydroxy-N-(methyl-d3)-1-(6-methyl-4-(trifluoromethyppyridin-2-yl)-5-oxopyrrolidine-2-carboxamide hemihydrate (Form B) (Example 2), i.e. a compound of formula (IB).

11. The compound of formula (IB) according to claim 10, which is characterised by any one or more of the following parameters:
(i) an XRPD pattern substantially as shown in FIG. 4;
(ii) peaks at the same diffraction angles (2θ) of the XRPD pattern shown in FIGS. 4 and optionally wherein the peaks have the same relative intensity as the peaks shown in FIG. 4;
(iii) major peaks at diffraction angles (2θ) and intensities as those shown in the XRPD pattern in FIG. 4;
(iv) an XRPD pattern having peaks at 5.1±0.5°, 8.7±0.5°, 10.1±0.5°, 12.2±0.5°, 12.7±0.5°, 14.2±0.5°, 15.1±0.5°, 16.5±0.5°, 17.1±0.5°, 18.8±0.5°, 20.2±0.5°, 22.4±0.5° and 22.9±0.5° (20, 1d.p);
(v) an XRPD pattern having peaks at 5.1±0.2°, 8.7±0.2°, 10.1±0.2°, 12.2±0.2°, 12.7±0.2°, 14.2±0.2°, 15.1±0.2°, 16.5±0.2°, 17.1±0.2°, 18.8±0.2°, 20.2±0.2°, 22.4±0.2° and 22.9±0.2° (20, 1 d.p);
(vi) an XRPD pattern having peaks at 5.1±0.1°, 8.7±0.1°, 10.1±0.1°, 12.2±0.1°, 12.7±0.1°, 14.2±0.1°, 15.1±0.1°, 16.5±0.1°, 17.1±0.1°, 18.8±0.1°, 20.2±0.1°, 22.4±0.1° and 22.9±0.1° (20, 1 d.p);
(vii) an XRPD pattern having peaks at 5.1, 8.7, 10.1, 12.2, 12.7, 14.2, 15.1, 16.5, 17.1, 18.8, 20.2, 22.4 and 22.9 (20, 1d.p);
(viii) an XRPD pattern having peaks as set out in the below table:

| | Relative intensity, |
|---|---|
| Angle, °2θ | %* |
| 5.1 | 84.4 |
| 8.7 | 24.7 |
| 10.1 | 100.0 |
| 12.2 | 66.4 |
| 12.7 | 32.3 |
| 14.2 | 27.0 |
| 15.1 | 25.8 |
| 16.5 | 36.4 |
| 17.1 | 22.0 |
| 18.8 | 26.7 |
| 20.2 | 28.0 |
| 22.4 | 24.3 |
| 22.9 | 48.7 |

*Peaks with relative intensity of less than 20% are not reported.
(ix) a differential scanning calorimetry (DSC) onset temperature of 80.25° C.±0.5° C. (such as 80.25° C.±0.2° C., in particular 80.25° C.±0.1° C., more particularly 80.25° C.);
15 (x) a differential scanning calorimetry (DSC) peak temperature of 95.02° C.±0.5° C. (such as 95.02° C.±0.2° C., in particular 95.02° C.±0.1° C., more particularly 95.02° C.);
(xi) a differential scanning calorimetry (DSC) thermogram as depicted in FIG. 5;
(xii) a thermogravimetric peak mass loss at a temperature of 259.71° C.±0.5° C. (such as 259.71° C.±0.2° C., in particular 259.71° C.±0.1° C., more particularly 259.71° C.); and/or
(xiii) a thermogravimetric analysis (TGA) thermogram as depicted in FIG. 6.

12. A pharmaceutical composition comprising a compound of formula (I) according to any of claims 7 to 11, in combination with one or more therapeutic agents.

13. A compound of formula (I) according to any of claims 7 to 11 for use in therapy.

14. A compound of formula (I) according to any of claims 7 to 11 for use in the prophylaxis or treatment of cancer.

15. A process for preparing a compound of formula (XX) as defined in claim 1, which comprises reacting a compound of formula (XVIII): 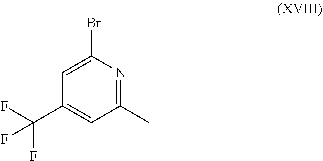
with a compound of formula (XIX):

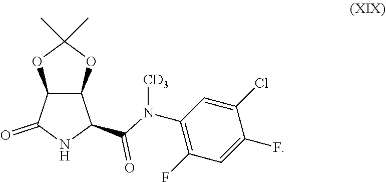 [SMILES: [2H]C([2H])([2H])N(C(=O)[C@H]1NC(=O)[C@H]2OC(C)(C)O[C@@H]12)c1cc(Cl)c(F)cc1F]

16. The process according to claim 15, which comprises a suitable catalyst, such as a copper catalyst, in particular copper (I) iodide, and a suitable ligand, such as N,N′-dimethylethylenediamine.

17. A process for preparing a compound of formula (XVI): 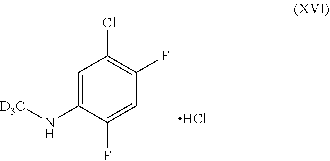 [SMILES: [2H]C([2H])([2H])Nc1cc(Cl)c(F)cc1F ; Cl]
which comprises reacting a compound of formula (XV): 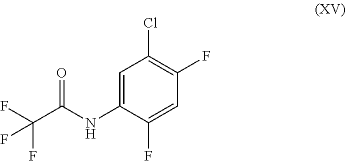 [SMILES: O=C(Nc1cc(Cl)c(F)cc1F)C(F)(F)F]
in a single vessel with methyl-d3 iodide in the presence of an inorganic base, such as potassium carbonate, followed by treatment with potassium acetate and further addition of an inorganic base, such as potassium carbonate.

18. A process for preparing a compound of formula (XIII): 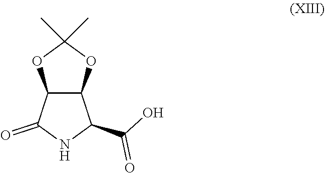
which comprises the use of a compound of formula (II): 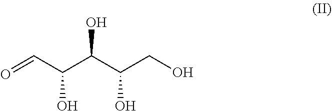
as the starting material.

19. The process according to claim 18, which comprises the following steps:

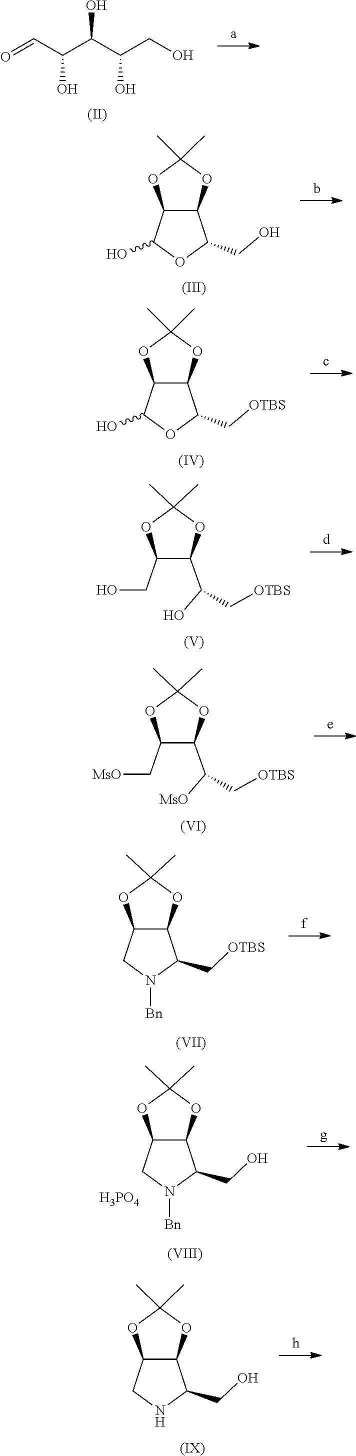

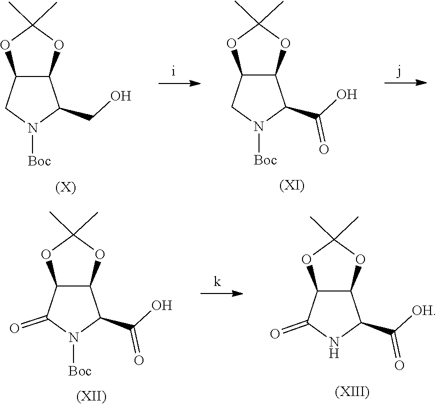

20. A process for preparing a compound of formula (XII) as defined in claim 19, which comprises reacting a compound of formula (XI) as defined in claim 19, with suitable oxidants, such as ruthenium dioxide and sodium periodate.

21. A process for preparing a compound of formula (XI) as defined in claim 19, which comprises reacting a compound of formula (X) as defined in claim 19, with suitable oxidants, such as ruthenium trichloride and sodium periodate.

22. A process for preparing a compound of formula (XII) as defined in claim 19, which comprises reacting a compound of formula (X) as defined in claim 19, with suitable oxidants, such as ruthenium trichloride and sodium periodate.

23. A process for preparing a compound of formula (VIII) as defined in claim 19, which comprises reacting a compound of formula (VII) as defined in claim 19, with a suitable acid, such as phosphoric acid.

24. A process for preparing a compound of formula (V) as defined in claim 19, which comprises the use of a compound of formula (II) as defined in claim 19, as the starting material.

25. The process according to claim 24, which comprises the following steps: [chemical structure]
